(19) United States
(12) Patent Application Publication
(10) Pub. No.: US 2026/0076561 A1
(43) Pub. Date: Mar. 19, 2026

(54) METHODS AND SYSTEMS FOR IDENTIFICATION OF POTENTIAL EYE STRAIN ISSUES USING VIRTUAL REALITY

(71) Applicant: Zenni Optical, Inc., Novato, CA (US)

(72) Inventors: Steven LEE, Barrington, IL (US); Julia ZHEN, Novato, CA (US); ChyrSong TING, Novato, CA (US); Matthew James GOLINO, Brookhaven, GA (US); Justin Paul DEMPSEY, Ottawa (CA); Jeffrey Joseph FILLINGHAM, Dartmouth (CA)

(21) Appl. No.: 18/885,526

(22) Filed: Sep. 13, 2024

(51) Int. Cl. A61B 3/10; A61B 3/00

(52) U.S. Cl. CPC A61B 3/10; A61B 3/005

(57) ABSTRACT

A virtual reality (VR) system can be implemented to identify potential eye strain issues through prolonged engagement. The system employs an electronic device featuring a high-resolution VR headset with integrated eye-tracking sensors. It generates a VR user interface corresponding to a three-dimensional virtual environment and renders it on the VR headset. Within this environment, the system presents a series of progressively challenging visual tasks. Throughout these tasks, the system continuously monitors the user's eye movements and behavior using the eye-tracking sensors. The collected data is then evaluated for indicators of eye strain, potentially allowing for early detection and intervention in cases of visual discomfort during extended VR use.

### TECHNICAL FIELD

The present inventions relate to vision test technology. More specifically, methods, systems, devices, and non-statutory computer-readable storage media are applied to implement vision testing in an extended reality environment to identify potential eye strain issues through prolonged engagement.

### BACKGROUND

Traditional visual assessment methods have been the cornerstone of evaluating eye health and vision for many years. These methods are typically conducted in clinical environments, where specialized equipment and standardized procedures are used to ensure accurate and reliable results. The parameters for these assessments are generally fixed, reflecting the controlled nature of the clinical setting.

Over time, these techniques have become the accepted standard for diagnosing and monitoring visual conditions, forming the basis of routine eye care practices in medical offices, hospitals, and specialized eye care facilities. Despite their widespread use, these methods have traditionally been limited to professional settings, where they can be conducted under the supervision of trained healthcare providers using dedicated equipment.

### SUMMARY

The present disclosure relates to innovative methods and systems that can revolutionize vision care, making vision testing and other exams more accessible and affordable for patients. Additionally, it is contemplated that the principles and features of the present disclosure can be implemented in numerous other applications of display technology, including headsets, heads-up displays, and other micro-displays (e.g., microLED and microOLED) to address challenges and limitations inherent in such products and their uses.

In accordance with at least some embodiments disclosed herein is the realization that traditional methods for visual assessment do not allow for dynamic adjustment of test parameters, leading to less accurate assessments, nor can they be implemented to test eyes and vision at home using household devices in a consistent and environment-locked manner.

Some embodiments are directed to a method of implementing a virtual vision test at an electronic device including a head-mounted display (HMD) and a camera. The method includes executing a user application configured to enable the virtual vision test; generating a virtual reality (VR) user interface corresponding to a three-dimensional (3D) virtual environment; focusing the camera on an eye area of a user wearing the electronic device; displaying, on the user interface, a visual stimulus corresponding to the virtual vision test; while displaying the visual stimulus, in real time, capturing a sequence of eye images using the camera of the electronic device; determining eye movement information including a temporal sequence of eyeball positions based on the sequence of eye images; and comparing the visual stimulus and the eye movement information to determine an eye health condition.

In some embodiments, a user application can be implemented by a head-mounted display configured to create a customized extended reality (XR) environment for a user engaged on an XR information platform. Products may be rendered for the user in a three-dimension format in the XR environment, thereby facilitating eyewear selection and fitting. The XR can be an umbrella term encapsulating Augmented Reality (AR), Virtual Reality (VR), Mixed Reality (MR), and everything in between. In this application, any embodiments that apply a VR system can be implemented using an AR or MR system as well.

Some embodiments are directed to a method of implementing a virtual reality (VR) system for testing light sensitivity and prescribing customized LCD tinted lenses. The method is performed at an electronic device including a head-mounted display and eye-tracking sensors. The method includes generating a VR user interface corresponding to a three-dimensional virtual environment and rendering the VR user interface on the head-mounted display. The method also includes simulating various lighting conditions sequentially in the VR user interface. While simulating the various lighting conditions, in real time, the method continuously tracks, using the eye-tracking sensors, gaze direction, blink rate, squinting, and pupillary responses to the simulated lighting conditions. The method also includes evaluating the tracked data for light sensitivity performance. In this way, the method enables comprehensive assessment of an individual's light sensitivity in a controlled, immersive environment, facilitating the prescription of customized LCD tinted lenses tailored to the user's specific visual needs.

Some embodiments are directed to a method of implementing a virtual reality (VR) system for recommending lens tints through an interactive vision sensitivity test. The method is performed at an electronic device including a head-mounted display (HMD) and eye-tracking sensors. The method includes generating a VR user interface corresponding to a three-dimensional virtual environment and rendering the VR user interface on the head-mounted display. The method also includes simulating various lighting conditions and glare levels sequentially in the VR user interface. While simulating the various lighting conditions and glare levels, in real time, the method continuously tracks, using the eye-tracking sensors, user responses to the simulated lighting conditions and glare levels. The method also includes evaluating the tracked data for vision sensitivity performance. In this way, the method enables a comprehensive and interactive assessment of a user's vision sensitivity under various lighting and glare conditions in a controlled, immersive environment, facilitating the recommendation of personalized lens tints based on the user's specific visual responses and needs.

Some embodiments are directed to a method of implementing a virtual reality (VR) system for evaluating color perception. The method is performed at an electronic device including a head-mounted display (HMD) and eye-tracking sensors. The method includes generating a VR user interface corresponding to a three-dimensional virtual environment and rendering the VR user interface on the head-mounted display. The method also includes simulating various color-coded challenges and puzzles under varying luminosities and backgrounds in the VR user interface. While simulating the color-coded challenges and puzzles, in real time, the method continuously tracks, using the eye-tracking sensors, user responses to the simulated challenges and puzzles. The method also includes evaluating the tracked data for color perception performance. In this way, the method enables a comprehensive and dynamic assessment of color perception abilities under diverse visual conditions in an immersive, controlled environment. By utilizing interactive challenges and puzzles, the system can evaluate nuanced aspects of color perception, potentially uncovering subtle deficiencies or strengths that might not be apparent in traditional color vision tests.

Some embodiments are directed to a method of implementing a virtual reality (VR) system for evaluating color perception. The method is performed at an electronic device including a head-mounted display (HMD) and eye-tracking sensors. The method includes generating a VR user interface corresponding to a three-dimensional virtual environment and rendering the VR user interface on the head-mounted display. The method also includes simulating various color perception tasks under varying luminosities and backgrounds in the VR user interface. While simulating the color perception tasks, in real time, the method continuously tracks, using the eye-tracking sensors, user responses to the simulated tasks. The method also includes evaluating the tracked data for color perception performance. In this way, the method enables a comprehensive and dynamic assessment of color perception abilities under diverse visual conditions in an immersive, controlled environment. By utilizing a range of color perception tasks and varying environmental factors, the system can evaluate, for example, nuanced aspects of color vision, potentially uncovering subtle deficiencies or strengths that might not be apparent in traditional color vision tests.

Some embodiments are directed to a method of implementing a virtual reality (VR) system for evaluating color perception, with a specific focus on color wavelength sensitivity. The method is performed at an electronic device including a head-mounted display (HMD) and eye-tracking sensors. The method includes generating a VR user interface corresponding to a three-dimensional virtual environment and rendering the VR user interface on the head-mounted display. The method also includes simulating various color wavelength tasks in the VR user interface. While simulating the color wavelength tasks, in real time, the method continuously tracks, using the eye-tracking sensors, user responses to the simulated tasks. The method also includes evaluating the tracked data for color wavelength sensitivity performance. In this way, the method enables a precise and comprehensive assessment of an individual's sensitivity to specific color wavelengths in an immersive, controlled environment. By utilizing specialized color wavelength tasks and advanced eye-tracking technology, the system can evaluate nuanced aspects of color perception at the wavelength level, potentially uncovering subtle variations in color sensitivity that might not be detected by conventional color vision tests.

Some embodiments are directed to a method of implementing a virtual reality (VR) system for testing and recommending adaptive eyewear for color blindness. The method is performed at an electronic device including a head-mounted display (HMD) and eye-tracking sensors. The method includes generating a VR user interface corresponding to a three-dimensional virtual environment and rendering the VR user interface on the head-mounted display. The method also includes simulating various real-world scenarios in the VR user interface. While simulating the real-world scenarios, in real time, the method continuously tracks, using the eye-tracking sensors, user responses to the simulated scenarios. The method also includes evaluating the tracked data for color perception performance. In this way, the method enables a comprehensive and realistic assessment of color vision deficiencies in simulated everyday situations, providing a basis for recommending personalized adaptive eyewear. By utilizing a range of real-world scenarios and advanced eye-tracking technology, the system can evaluate the effectiveness of different adaptive eyewear options in improving color perception for individuals with color blindness.

Some embodiments are directed to a system for implementing a virtual eye test. The system includes a head-mounted display including a display and one or more cameras. The system also includes one or more processors and memory storing one or more programs configured to be executed by the one or more processors. The one or more programs includes instructions for a user interface module configured to generate a virtual reality (VR) user interface corresponding to a three-dimensional virtual environment. The one or more programs also includes instructions for a rendering module configured to render the VR user interface on the HMD. The one or more programs also includes instructions for a simulation module configured to simulate one or more scenarios in the VR user interface. The one or more programs also includes instructions for a tracking module configured to continuously track, using at least one of the one or more cameras and/or eye-tracking sensors, eye movements and/or responses to visual stimuli presented in the one or more scenarios. The one or more programs also includes instructions for an evaluation module configured to analyze user interactions and system performance to determine and/or measure at least one of: light sensitivity performance, vision sensitivity performance, color sensitivity performance, color perception performance, and/or color wavelength sensitivity performance.

In another aspect, a non-transitory computer readable storage medium is provided, according to some embodiments. The medium stores one or more programs for execution by one or more processors of a computer system, the one or more programs including instructions for performing any of the methods described herein.

In another aspect, an electronic device is provided, according to some embodiments. The electronic device includes an HMD, a camera and/or eye-tracking sensors, one or more processors, and memory for storing one or more programs for execution by the one or more processors, the one or more programs including instructions for performing any of the methods described herein.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE FIGURES

Various features of illustrative embodiments of the inventions are described below with reference to the drawings. The illustrated embodiments are intended to illustrate, but not to limit, the inventions.

FIG. 1 is an example data processing environment having one or more servers communicatively coupled to one or more computer devices (e.g., includes a headset device), in accordance with some embodiments.

FIG. 2 is an environment in which a computer device (e.g., a headset device) is applied to facilitate visual assessment or eyewear fitting, in accordance with some embodiments.

FIG. 3 is a block diagram of a computer system (e.g., including a headset device) configured to implement vision assessment or eyewear fitting, in accordance with some embodiments.

FIG. 4 is a block diagram of a machine learning system for training and applying machine learning models (e.g., for glass making), in accordance with some embodiments.

FIG. 5A is a structural diagram of an example neural network applied to process input data in a machine learning model, in accordance with some embodiments, and FIG. 5B is an example node in the neural network, in accordance with some embodiments.

FIG. 6A is an example “tumbling E” chart applied in a visual acuity test, and FIGS. 6B-6E are example patterns applied in an astigmatism test, a stereopsis test, a visual field test, and a color blindness test, in accordance with some embodiments.

FIG. 7 is another example visual pattern applied to test visual acuity and astigmatism, in accordance with some embodiments.

FIGS. 8A-8D include four diagrams of example graphical user interfaces rendered to determine a visual acuity score in a virtual environment created by a headset device, in accordance with some embodiments.

FIGS. 9A-9C include three diagrams of example graphical user interfaces rendered to determine a nearsighted or farsighted power in a virtual environment created by a headset device, in accordance with some embodiments.

FIGS. 10A-10F include six diagrams of example graphical user interfaces rendered to determine eye stigmatism in a virtual environment created by a headset device, in accordance with some embodiments.

FIGS. 11A and 11B are diagrams showing an example vision test system, in accordance with some embodiments.

FIGS. 12A-12H show a flow diagram of an example process for implementing a virtual reality (VR) system that adjusts visual complexity based on real-time eye fatigue monitoring, according to some embodiments.

FIGS. 13A-13G show a flow diagram of an example process for real-time visual health monitoring during extended use, according to some embodiments.

FIGS. 14A-14F show a flow diagram of an example process for vision testing and eye health monitoring, according to some embodiments.

FIGS. 15A-15H show a flow diagram of an example process for identifying potential eye strain issues through prolonged engagement, according to some embodiments.

FIGS. 16A-16G show a flow diagram of an example process for evaluating vision during digital device use and identifying blue light sensitivity, according to some embodiments.

FIGS. 17A-17G show a flow diagram of an example process for testing cognitive load and mental fatigue effects on vision, according to some embodiments.

FIGS. 18A-18G show a flow diagram of an example process for evaluating visual discomfort in users with eye strain sensitivity, according to some embodiments.

FIG. 19 is a schematic diagram showing an example vision test, in accordance with some embodiments.

FIGS. 20A, 20B and 20C show illustrations of example visual scenarios for a VR eye fatigue monitoring and adjustment system, according to some embodiments.

FIG. 20D is a block diagram of example components for a VR-based visual fatigue assessment and mitigation system, according to some embodiments.

FIG. 21A shows illustrations of example scenarios for a VR real-time visual health monitoring system for extended use, according to some embodiments.

FIG. 21B is a block diagram of example components for a VR real-time visual health monitoring system for extended use, according to some embodiments.

FIG. 22A shows an example view of a person wearing (i) a high-resolution VR headset with eye-tracking sensors, (ii) contact lens sensor, and (iii) handheld device, according to some embodiments.

FIG. 22B is a block diagram of example components for a VR-based vision testing and eye health monitoring system, according to some embodiments.

FIG. 23A shows illustrations of example scenarios for a VR eye strain identification system through prolonged engagement, according to some embodiments.

FIG. 23B is a block diagram of example components for a VR-based eye strain identification system, according to some embodiments.

FIG. 24A shows an example view of a person navigating websites on a screen that shows a lot of blue light, for a VR-based blue light sensitivity evaluation system for digital device use, according to some embodiments.

FIG. 24B is a block diagram of example components for a VR-based blue light sensitivity assessment system, according to some embodiments.

FIG. 25A shows an example view of a person engaged in a high-stress multitasking scenario involving solving visual puzzles, for a VR-based cognitive load and mental fatigue testing system, according to some embodiments.

FIG. 25B is a block diagram of example components 2502 for a VR-based cognitive load and mental fatigue assessment system, according to some embodiments.

FIG. 26A shows an example view of a person engaged in a visually demanding task for dynamic tracking of fast-moving objects, for a VR-based eye strain identification system for visual discomfort evaluation, according to some embodiments.

FIG. 26B is a block diagram of example components for a VR-based eye strain assessment and monitoring system, according to some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for case of understanding.

Moreover, various aspects of the present disclosure can be implemented in combination with aspects of other virtual-reality technology developed by the present applicant, for example, in copending U.S. Patent App. Nos. 63/560,623 (137034-5002), filed on Mar. 1, 2024, 63/569,095 (137034-5005), filed on Mar. 23, 2024, 63/642,571 (137034-5007), filed on May 3, 2024, 63/642,583 (137034-5009), filed on May 3, 2024, 63/642,593 (137034-5010), filed on May 3, 2024, 63/642,604 (137034-5011), filed on May 3, 2024, 63/644,457 (137034-5012), filed on May 8, 2024, Ser. No. 18/759,641 (137034-5018), filed on Jun. 28, 2024, Ser. No. 18/791,203 (137034-5036), filed on Jul. 31, 2024, Ser. No. 18/827,546 (137034-5050), filed Sep. 6, 2024, and Ser. No. 18/827,588 (137034-5070), filed Sep. 6, 2024, Ser. No. 18/819,311 (137034-5029), filed Aug. 29, 2024, Ser. No. 18/820,121 (137034-5047), filed Aug. 29, 2024, Ser. No. 18/820,140 (137034-5063), filed Aug. 29, 2024, App. No. TBD (137034-5084), filed Sep. 13, 2024, the entireties of each of which is incorporated herein by reference. Aspects of these copending cases can be implemented in combination with some embodiments disclosed herein, whether in addition to features thereof or as an alternative to a particular feature of an embodiment disclosed herein.

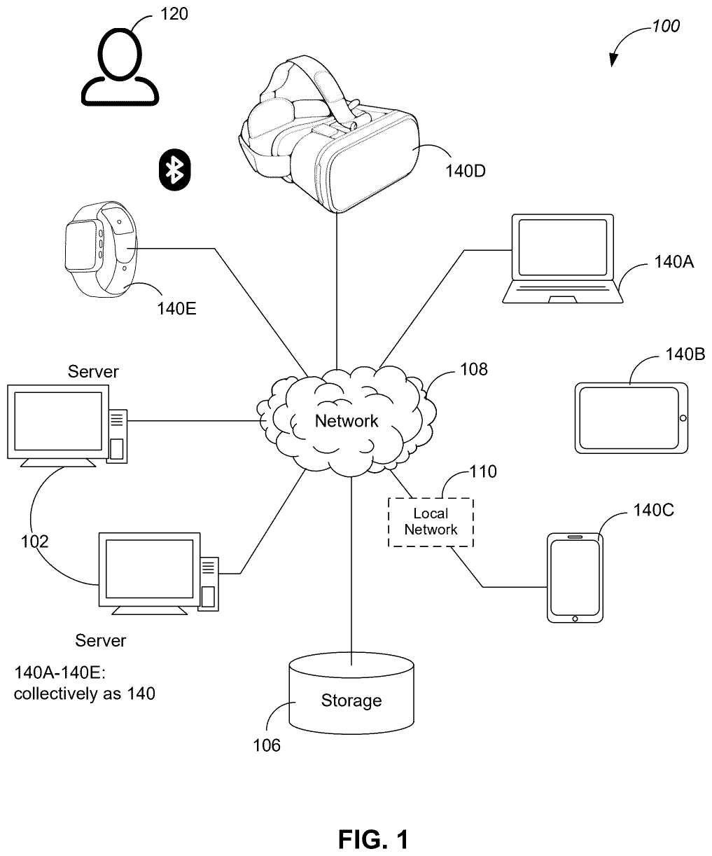

FIG. 1 is an example data processing environment 100 having one or more servers 102 communicatively coupled to one or more computer devices 140 (e.g., includes a headset device 140D), in accordance with some embodiments. The one or more computer devices 140 are electronic devices having computational capabilities, and may be, for example, desktop computers 140A, tablet computers 140B, mobile phones 140C, or intelligent, multi-sensing, network-connected home devices (e.g., a depth camera, a visible light camera). In some embodiments, the one or more computer devices 140 include a headset device 140D (also called a head-mounted display 140D) configured to render extended reality content. In some embodiments, the one or more computer devices 140 include a wireless wearable device 140E (e.g., a smart watch, a fitness band) configured to track health data (e.g., heart rate, quality of sleep) and activity data (e.g., steps walked, stairs climbed) of a user wearing the device 140E. Each computer device 140 can collect data or user inputs, executes user applications, and present outputs on its user interface. The collected data or user inputs can be processed locally at the computer device 140 and/or remotely by the server(s) 102. The one or more servers 102 provides system data (e.g., boot files, operating system images, and user applications) to the computer devices 140, and in some embodiments, processes the data and user inputs received from the computer device(s) 140 when the user applications are executed on the computer devices 140. In some embodiments, the data processing environment 100 further includes a storage 106 for storing data related to the servers 102, computer devices 140, and applications executed on the computer devices 140. For example, storage 106 may store video content, static visual content, and/or audio data.

The one or more servers 102 can enable real-time data communication with the computer devices 140 that can be remote from each other or from the one or more servers 102. Further, in some embodiments, the one or more servers 102 can implement data processing tasks that are not completed locally by the computer devices 140. For example, the computer devices 140 include a game console (e.g., the headset device 140D) that executes an interactive online gaming application. The game console receives a user instruction and sends it to a game server 102 with user data. The game server 102 generates a stream of video data based on the user instruction and user data and provides the stream of video data for display on the game console and other computer devices that can be engaged in the same game session with the game console.

The one or more servers 102, one or more computer devices 140, and storage 106 can be communicatively coupled to each other via one or more communication networks 108, which are the medium used to provide communications links between these devices and computers connected together within the data processing environment 100. The one or more communication networks 108 may include connections, such as wire, wireless communication links, or fiber optic cables. Examples of the one or more communication networks 108 include local area networks (LAN), wide area networks (WAN) such as the Internet, or a combination thereof. The one or more communication networks 108 are, optionally, implemented using any known network protocol includes various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VOIP), Wi-MAX, or any other suitable communication protocol. A connection to the one or more communication networks 108 may be established either directly (e.g., using 1G/4G connectivity to a wireless carrier), or through a network interface 110 (e.g., a router, switch, gateway, hub, or an intelligent, dedicated whole-home control node), or through any combination thereof. As such, the one or more communication networks 108 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other electronic systems that route data and messages.

In some embodiments, the headset device 140D can be communicatively coupled to a data processing environment 100. The headset device 140D includes one or more cameras (e.g., a visible light camera, a depth camera), a microphone, a speaker, one or more inertial sensors (e.g., gyroscope, accelerometer), and a display. In some situations, the camera captures hand gestures of a user wearing the headset device 140D. In some situations, the microphone records ambient sound includes user's voice commands.

In some embodiments, the headset device 140D is communicatively coupled to one or more servers 102 and enables a centralized vision test management platform with the one or more servers 102. This vision test management platform may aggregate data (e.g., visual stimuli 338, sensor data 342, vision test results 344) from a plurality of user accounts associated with a plurality of users, analyze the aggregated data, and track vision health trends for individual users or user groups. In some embodiments, data are communicated between a headset device 140D and a server 102 in an encrypted format. In some embodiments, the vision test management platform is coupled to a global health database storing epidemiological data and configured to cross-reference the data collected from its user accounts with the epidemiological data to identify an emerging pattern and a public health concern. For example, a teenager's vision data was collected and analyzed during an extended duration of time (e.g., 10 years) to identify an individual vision development trend and cross-referenced with an average vision development trend extracted from the global health database. A doctor can rely on a cross-referencing result to determine whether the individual vision development trend is normal or whether the teenager's eyesight drops faster than average teenagers. As such, various embodiments of the vision test management platform integrate biometric data and global health analytics and provides a secure, personalized, and interactive environment for vision testing, which improves precision and user experience of vision assessments and contributes to broader public health monitoring and research initiatives.

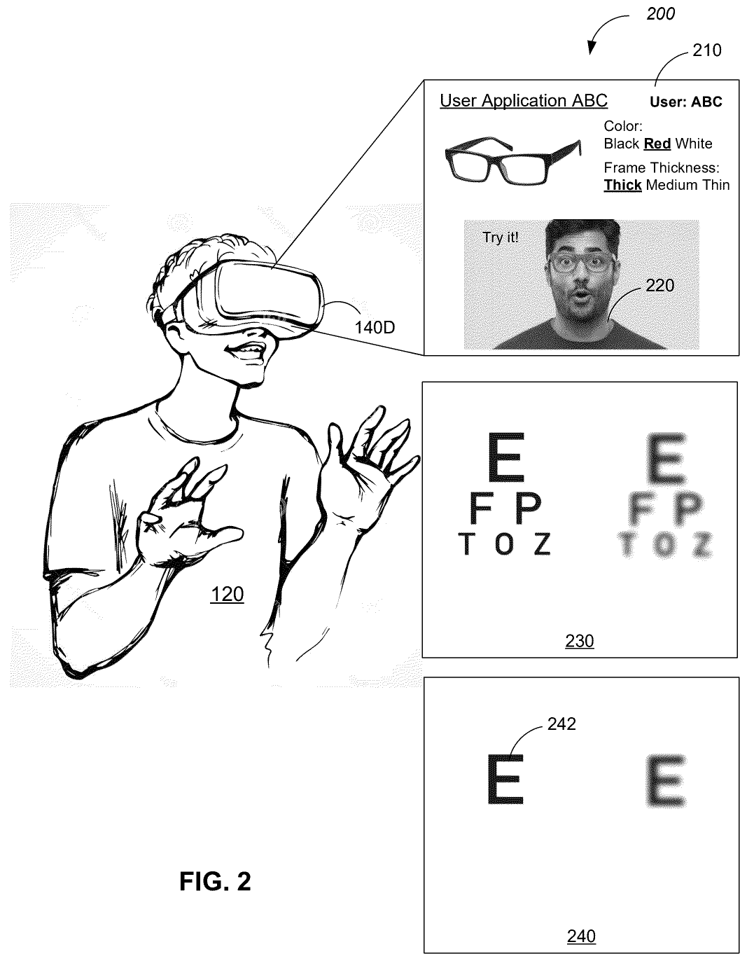

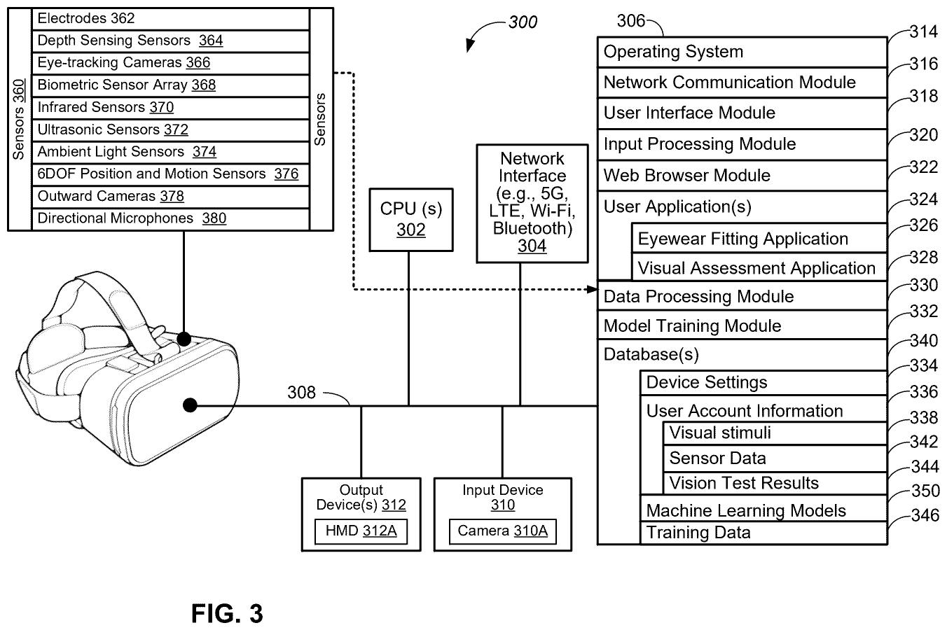

FIG. 2 is an environment 200 in which a computer device 140 (e.g., a headset device 140D) is applied to facilitate visual assessment or eyewear fitting, in accordance with some embodiments. The XR headset device 140D may be communicatively coupled within the data processing environment 100. The XR headset device 140D may include one or more cameras (e.g., a visible light camera, a depth camera), a microphone, a speaker, one or more inertial sensors (e.g., gyroscope, accelerometer), and a display. In some situations, the camera captures hand gestures of a user wearing the XR headset device 140D. In some situations, the microphone records ambient sound includes user's voice commands. The XR headset device 140D may execute a client-side eyewear fitting application 326 or a client-side visual assessment application 328 (FIG. 3) via a user account associated with a user 120 (e.g., an optometrist user, an optician user, a patient user). In some embodiments, a computer device 140 (e.g., a mobile phone 140C) distinct from the XR headset device 140D can be used to implement the client-side eyewear fitting application 326 or visual assessment application 328 (FIG. 3).

In some embodiments, a first user interface 210 can be displayed on a computer device 140 (e.g., the headset device 140D) associated with the user 120. In some embodiments, an eyewear can be tried on or displayed as being worn by a 2D or 3D image 220 of the user 120. The server 102 or computer device 140 receives, from the first user interface 210, a user feedback message indicating an issue, requesting further improvement, or confirming a fit. In some embodiments, a second user interface 230 can be displayed on a computer device 140 associated with the user 120. The second user interface 230 includes a plurality of optotypes (e.g., six optotypes E, F, P, T, O, and Z) having different sizes. In some embodiments, a third user interface 240 can be displayed on a computer device 140 associated with the user 120. The second user interface 230 can display a temporal sequence of optotypes having respective sizes. Each optotype of a corresponding size can be displayed at one time.

FIG. 3 is a block diagram of a computer system 300 (e.g., including a headset device 140D, a server, or a combination thereof) configured to implement vision assessment or eyewear fitting, in accordance with some embodiments. The computer system 300 typically, includes one or more processing units (CPUs) 302, one or more network interfaces 304, memory 306, and one or more communication buses 308 for interconnecting these components (sometimes called a chipset). The computer system 300 includes one or more input devices 310 that facilitate user input, such as a keyboard, a mouse, a voice-command input unit or microphone, a touch screen display, a touch-sensitive input pad, a gesture capturing camera, or other input buttons or controls. Furthermore, in some embodiments, the computer device 140 of the computer system 300 uses a microphone for voice recognition or an eye tracking camera 366 for tracking eyeball movement. In some embodiments, the computer device 140 includes one or more optical cameras (e.g., an RGB camera), scanners, or photo sensor units for capturing images. The computer system 300 also includes one or more output devices 312 that enable presentation of user interfaces 210 and display content includes one or more speakers and/or one or more visual displays.

The computer system 300 includes one or more sensors 360, which further includes one or more of: a plurality of electrodes 362, one or more depth sensing sensors 364, one or more eye tracking cameras 366, a biometric sensor array 368, one or more infrared sensors 370, one or more ultrasonic sensors 372, one or more ambient sensors 374, one or more motion sensors (e.g., six degree of freedom (6DOF) position and motion sensors 376, one or more outward camera 378, and one or more directional microphones 380. It is noted that the one or more sensors 360 are also included in the input device 310 and used to collect data to the computer system 300.

Memory 306 includes high-speed random-access memory, such as DRAM, SRAM, DDR RAM, or other random-access solid state memory devices; and, optionally, includes non-volatile memory, such as one or more magnetic disk storage devices, one or more optical disk storage devices, one or more flash memory devices, or one or more other non-volatile solid state storage devices. Memory 306, optionally, includes one or more storage devices remotely located from one or more processing units 302. Memory 306, or alternatively the non-volatile memory within memory 306, includes a non-transitory computer readable storage medium. In some embodiments, memory 306, or the non-transitory computer readable storage medium of memory 306, stores the following programs, modules, and data structures, or a subset or superset thereof:
Operating system 314 including procedures for handling various basic system services and for performing hardware dependent tasks;
Network communication module 316 for connecting each server 102 or computer device 140 to other devices (e.g., server 102, computer device 140, or storage 106) via one or more network interfaces 304 (wired or wireless) and one or more communication networks 108, such as the Internet, other wide area networks, local area networks, metropolitan area networks, and so on;
User interface module 318 for enabling presentation of information (e.g., a graphical user interface for application(s) 324, widgets, websites and web pages thereof, and/or games, audio and/or video content, text, etc.) at each computer device 140 via one or more output devices 312 (e.g., displays, speakers, etc.);
Input processing module 320 for detecting one or more user inputs or interactions from one of the one or more input devices 310 and interpreting the detected input or interaction;
Web browser module 322 for navigating, requesting (e.g., via HTTP), and displaying websites and web pages thereof includes a web interface for logging into a user account associated with a computer device 140 or another electronic device, controlling the computer device if associated with the user account, and editing and reviewing settings and data that are associated with the user account;
One or more user applications 324 for execution by the computer system 300 (e.g., games, social network applications, smart home applications, extended reality application, and/or other web or non-web-based applications for controlling another electronic device and reviewing data captured by such devices), where in some embodiments, an eyewear fitting application 326 can be executed to implement eyewear fitting, and has a plurality of user accounts associated with a plurality of users 120 (e.g., technician users and eyewear users), and in some embodiments, a visual assessment application 328 can be executed to evaluate eyesight of a patient user, and has a plurality of user accounts associated with a plurality of users 120 (e.g., an optometrist user, a patient user);
Data processing module 330 for processing data associated with the user applications 324, e.g., using machine learning models 350;
Model training Module 332 for obtaining training data 346 and training machine learning models 350; and
One or more databases 340 for storing at least data including one or more of:
   Device settings 334 including common device settings (e.g., service tier, device model, storage capacity, processing capabilities, communication capabilities, etc.) of the computer system 300;
   User account information 336 for the one or more user applications 324, e.g., user names, security questions, account history data, user preferences, and predefined account settings, where in some embodiments, the user account information 336 includes facial measurements and one or more virtual fitting parameters associated with associated with a user account of an eye fitting application 326, and in some embodiments, the user account information 336 includes visual stimuli 338, sensor data 342, and vision test results 344 associated with a user account of a visual assessment application 328; and
   Machine learning models 350 including parameters (e.g., weights, biases) used to implement vision test or select eyewear for eyewear users.

Each of the above identified elements may be stored in one or more of the previously mentioned memory devices and corresponds to a set of instructions for performing a function described above. The above identified modules or programs (i.e., sets of instructions) need not be implemented as separate software programs, procedures, modules or data structures, and thus various subsets of these modules may be combined or otherwise rearranged in various embodiments. In some embodiments, memory 306, optionally, stores a subset of the modules and data structures identified above. Furthermore, memory 306, optionally, stores additional modules and data structures not described above.

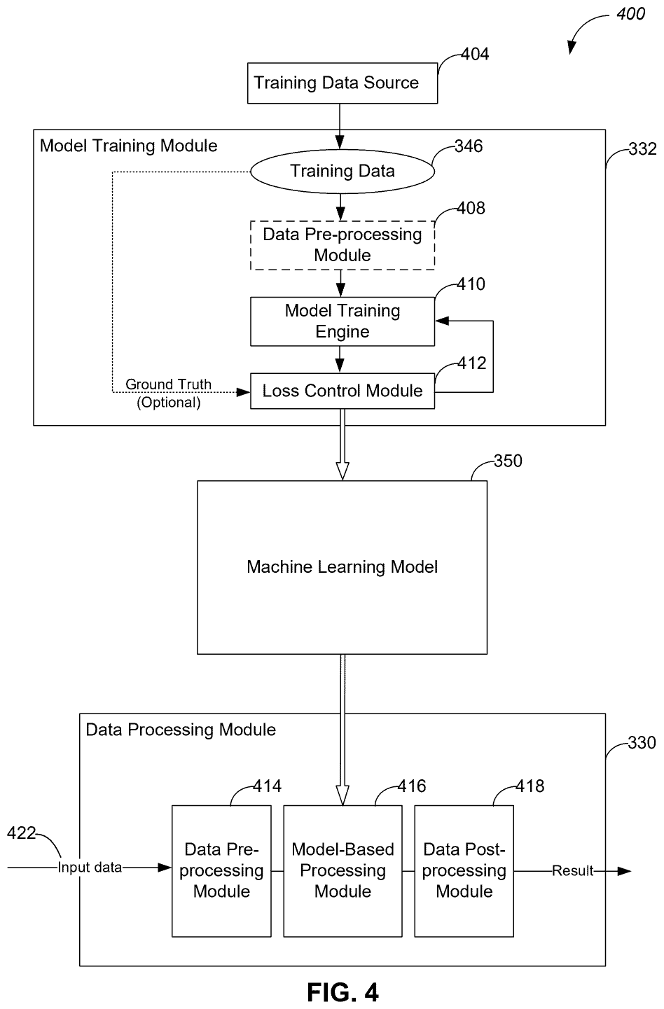

FIG. 4 is a block diagram of a machine learning system 400 for training and applying machine learning models 350 (e.g., for glass making), in accordance with some embodiments. The machine learning system 400 includes a model training module 332 establishing one or more machine learning models 350 and a data processing module 330 for processing input data 422 using the machine learning model 350. In some embodiments, both the model training module 332 and the data processing module 330 are located within a computer device 140 (e.g., a VR headset), while a training data source 404 provides training data 346 to the computer device 140. In some embodiments, the training data source 404 is the data obtained from the computer device 140 itself, from a server 102, from storage 106, or from another electronic device or computer device 140. Alternatively, in some embodiments, the model training module 332 is located at a server 102, and the data processing module 330 is located in a computer device 140. The server 102 trains the machine learning model 350 and provides the trained models 350 to the computer device 140 to process real-time input data 422 detected by the computer device 140. In some embodiments, the training data 346 provided by the training data source 404 include a standard dataset widely used to train machine learning models 350. The input data 422 further includes sensor data. Further, in some embodiments, a subset of the training data 346 is modified to augment the training data 346. The subset of modified training data is used in place of or jointly with the subset of training data 346 to train the machine learning models 350.

In some embodiments, the model training module 332 includes a model training engine 410, and a loss control module 412. Each machine learning model 350 is trained by the model training engine 410 to process corresponding input data 422 to implement a respective task. Specifically, the model training engine 410 receives the training data 346 corresponding to a machine learning model 350 to be trained and processes the training data to build the machine learning model 350. In some embodiments, during this process, the loss control module 412 monitors a loss function comparing the output associated with the respective training data item to a ground truth of the respective training data item. In these embodiments, the model training engine 410 modifies the machine learning models 350 to reduce the loss, until the loss function satisfies a loss criteria (e.g., a comparison result of the loss function is minimized or reduced below a loss threshold). The machine learning models 350 are thereby trained and provided to the data processing module 330 of a computer device 140 to process real-time input data 422 from the computer device 140.

In some embodiments, the model training module 402 further includes a data pre-processing module 408 configured to pre-process the training data 346 before the training data 346 is used by the model training engine 410 to train a machine learning model 350. For example, an image pre-processing module 408 is configured to format patients' eye images in the training data 346 into a predefined image format. For example, the preprocessing module 408 may normalize the images to a fixed size, resolution, or contrast level. In another example, an image pre-processing module 408 extracts a region of interest (ROI) corresponding to an eye area.

In some embodiments, the model training module 332 uses supervised learning in which the training data 346 is labelled and includes a desired output for each training data item (also called the ground truth in some situations). In some embodiments, the desirable output is labelled manually by people or labelled automatically by the model training model 332 before training. In some embodiments, the model training module 332 uses unsupervised learning in which the training data 346 is not labelled. The model training module 332 is configured to identify previously undetected patterns in the training data 346 without pre-existing labels and with little or no human supervision. Additionally, in some embodiments, the model training module 332 uses partially supervised learning in which the training data is partially labelled.

In some embodiments, the data processing module 330 includes a data pre-processing module 414, a model-based processing module 416, and a data post-processing module 418. The data pre-processing modules 414 pre-processes input data 422 based on the type of the input data 422. In some embodiments, functions of the data pre-processing modules 414 are consistent with those of the pre-processing module 408 and convert the input data 422 into a predefined data format that is suitable for the inputs of the model-based processing module 416. The model-based processing module 416 applies the trained machine learning model 350 provided by the model training module 332 to process the pre-processed input data 422. In some embodiments, the model-based processing module 416 also monitors an error indicator to determine whether the input data 422 has been properly processed in the machine learning model 350. In some embodiments, the processed input data is further processed by the data post-processing module 418 to create a preferred format or to provide additional information that can be derived from the processed input data. The data processing module 330 uses the processed input data to make eyewear glasses for a patient user.

Examples of the machine learning model 350 include, but are not limited to, an eye trajectory model, an eye position model, an ocular microtremor model, a response analysis model, a response analysis model, a biomedical data model, and medical information models.

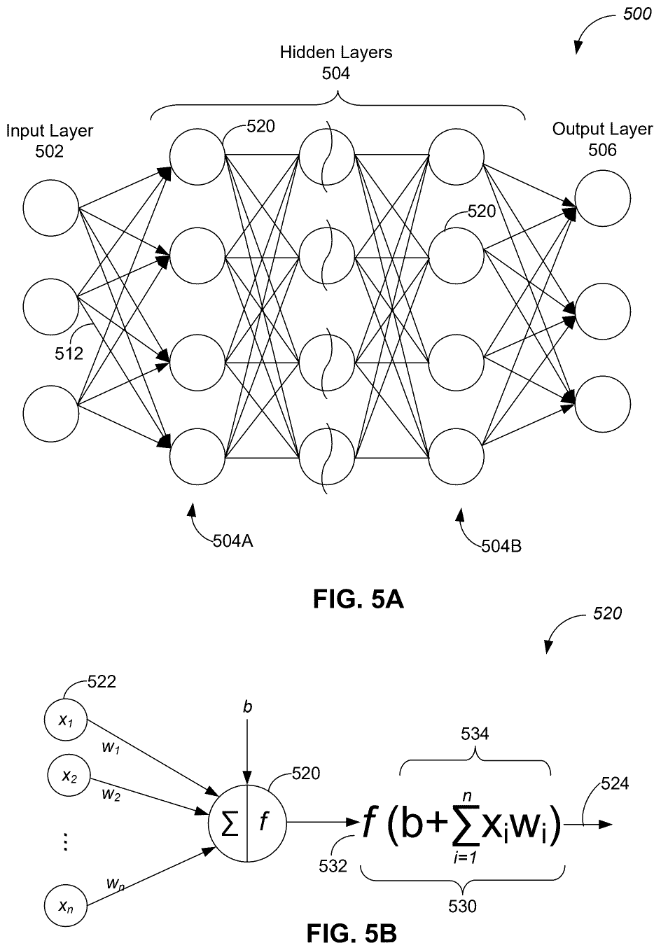

FIG. 5A is a structural diagram of an example neural network 500 applied to process input data in a machine learning model 350, in accordance with some embodiments, and FIG. 5B is an example node 520 in the neural network 500, in accordance with some embodiments. It should be noted that this description is used as an example only, and other types or configurations may be used to implement the embodiments described herein. The machine learning model 350 is established based on the neural network 500. A corresponding model-based processing module 416 applies the machine learning model 350 including the neural network 500 to process input data 422 that has been converted to a predefined data format. The neural network 500 includes a collection of nodes 520 that are connected by links 512. Each node 520 receives one or more node inputs 522 and applies a propagation function 530 to generate a node output 524 from the one or more node inputs. As the node output 524 is provided via one or more links 512 to one or more other nodes 520, a weight w associated with each link 512 is applied to the node output 524. Likewise, the one or more node inputs 522 are combined based on corresponding weights w₁, w₂, w₃, and w₄ according to the propagation function 530. In an example, the propagation function 530 is computed by applying a non-linear activation function 532 to a linear weighted combination 534 of the one or more node inputs 522.

The collection of nodes 520 is organized into layers in the neural network 500. In general, the layers include an input layer 502 for receiving inputs, an output layer 506 for providing outputs, and one or more hidden layers 504 (e.g., layers 504A and 504B) between the input layer 502 and the output layer 506. A deep neural network has more than one hidden layer 504 between the input layer 502 and the output layer 506. In the neural network 500, each layer is only connected with its immediately preceding and/or immediately following layer. In some embodiments, a layer is a “fully connected” layer because each node in the layer is connected to every node in its immediately following layer. In some embodiments, a hidden layer 504 includes two or more nodes that are connected to the same node in its immediately following layer for down sampling or pooling the two or more nodes. In particular, max pooling uses a maximum value of the two or more nodes in the layer for generating the node of the immediately following layer.

In some embodiments, a convolutional neural network (CNN) is applied in a machine learning model 350 to process input data. The CNN employs convolution operations and belongs to a class of deep neural networks. The hidden layers 504 of the CNN include convolutional layers. Each node in a convolutional layer receives inputs from a receptive area associated with a previous layer (e.g., nine nodes). Each convolution layer uses a kernel to combine pixels in a respective area to generate outputs. For example, the kernel may be to a 3×3 matrix including weights applied to combine the pixels in the respective area surrounding each pixel. Video or image data is pre-processed to a predefined video/image format corresponding to the inputs of the CNN. In some embodiments, the pre-processed video or image data is abstracted by the CNN layers to form a respective feature map. In this way, video and image data can be processed by the CNN for video and image recognition or object detection.

In some embodiments, a recurrent neural network (RNN) is applied in the machine learning model 350 to process input data 422. Nodes in successive layers of the RNN follow a temporal sequence, such that the RNN exhibits a temporal dynamic behavior. In an example, each node 520 of the RNN has a time-varying real-valued activation. It is noted that in some embodiments, two or more types of input data are processed by the data processing module 330, and two or more types of neural networks (e.g., both a CNN and an RNN) are applied in the same machine learning model 350 to process the input data jointly.

The training process is a process for calibrating all of the weights wi for each layer of the neural network 500 using training data 346 that is provided in the input layer 502. The training process typically includes two steps, forward propagation and backward propagation, which are repeated multiple times until a predefined convergence condition is satisfied. In the forward propagation, the set of weights for different layers are applied to the input data and intermediate results from the previous layers. In the backward propagation, a margin of error of the output (e.g., a loss function) is measured (e.g., by a loss control module 412), and the weights are adjusted accordingly to decrease the error. The activation function 532 can be linear, rectified linear, sigmoidal, hyperbolic tangent, or other types. In some embodiments, a network bias term b is added to the sum of the weighted outputs 534 from the previous layer before the activation function 532 is applied. The network bias b provides a perturbation that helps the neural network 500 avoid over fitting the training data. In some embodiments, the result of the training includes a network bias parameter b for each layer.

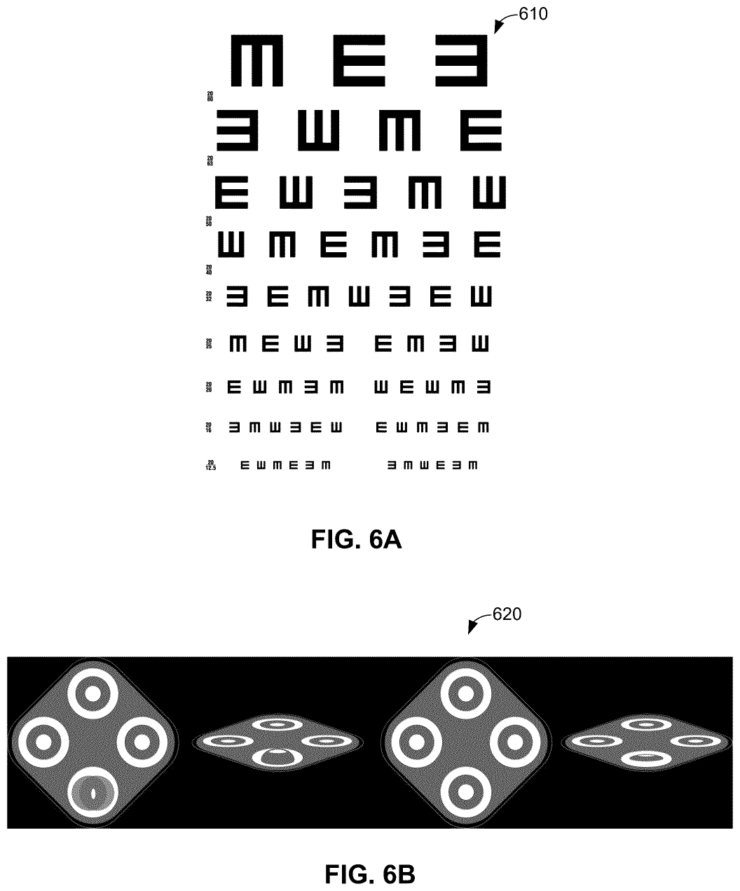

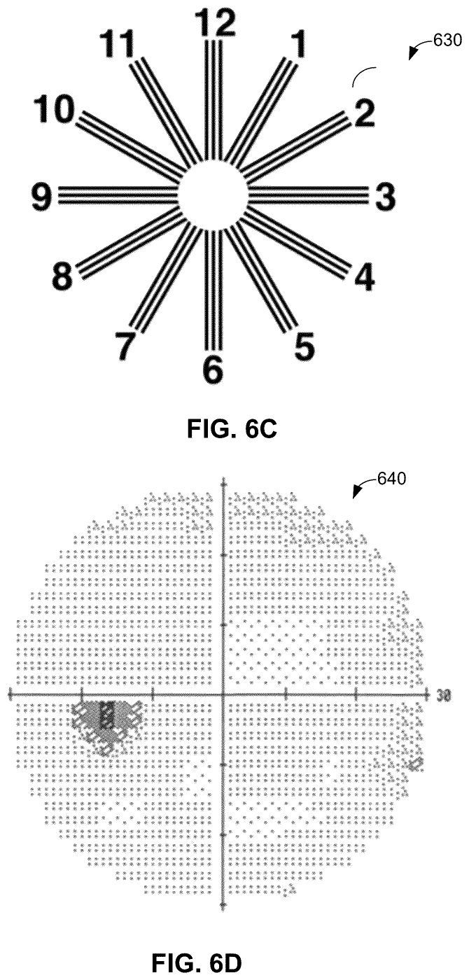

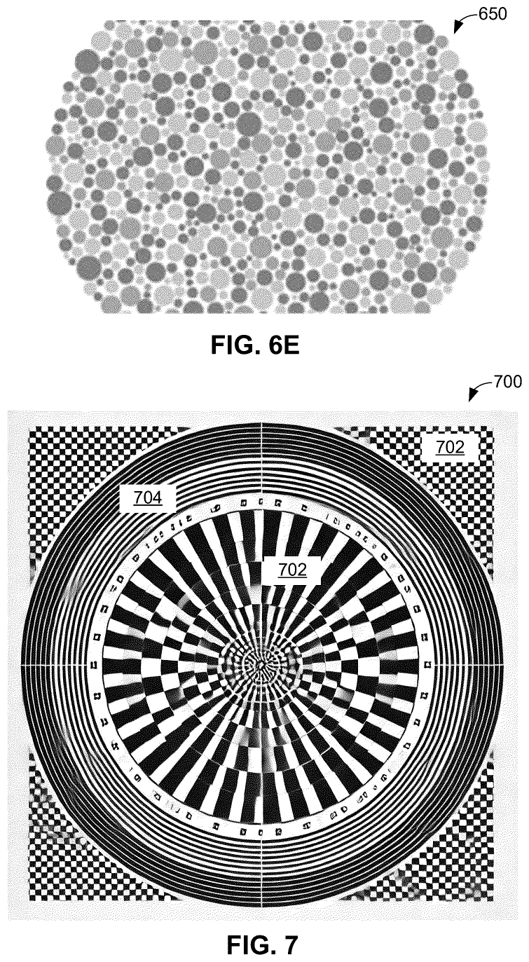

In some embodiments of the present disclosure, a vision test is implemented in a headset device 140D configured to display a user interface creating a three-dimensional (3D) virtual environment. Examples of a vision test implemented in the 3D virtual environment include, but are not limited to a visual acuity test, a visual field test, a visual depth test, a color blindness test, a retinoscopy, a test for stereopsis, a refraction test, an astigmatism test, and a contact lens exam. FIG. 6A is an example “tumbling E” chart 610 applied in a visual acuity test, in accordance with some embodiments. FIGS. 6B, 6C, 6D, and 6E are example patterns 620, 630, 640, and 650 applied in an astigmatism test, a stereopsis test, a visual field test, and a color blindness test, in accordance with some embodiments.

FIG. 7 is another example visual pattern 700 applied to test visual acuity and astigmatism, in accordance with some embodiments. The visual pattern 700 integrates a grid pattern 702 and concentric rings 704. The grid pattern 702 may include evenly spaced horizontal and vertical lines, creating a checkerboard pattern. The grid pattern 702 may be configured to identify distortions in straight lines, which can indicate issues with visual acuity and astigmatism. The concentric rings 704 may expand outward from a center of the visual pattern 700 and can assist in detecting radial distortions, which are common indicators of astigmatism. The visual pattern 700 may be depicted in high-contrast black and white, which ensures maximum clarity and reduces the potential for color-related distortions, making it easier to detect any visual impairment or defect.

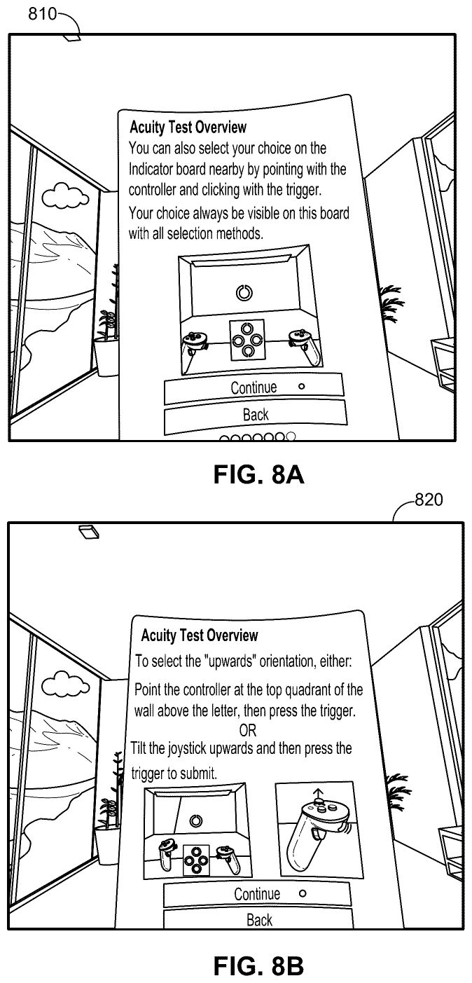

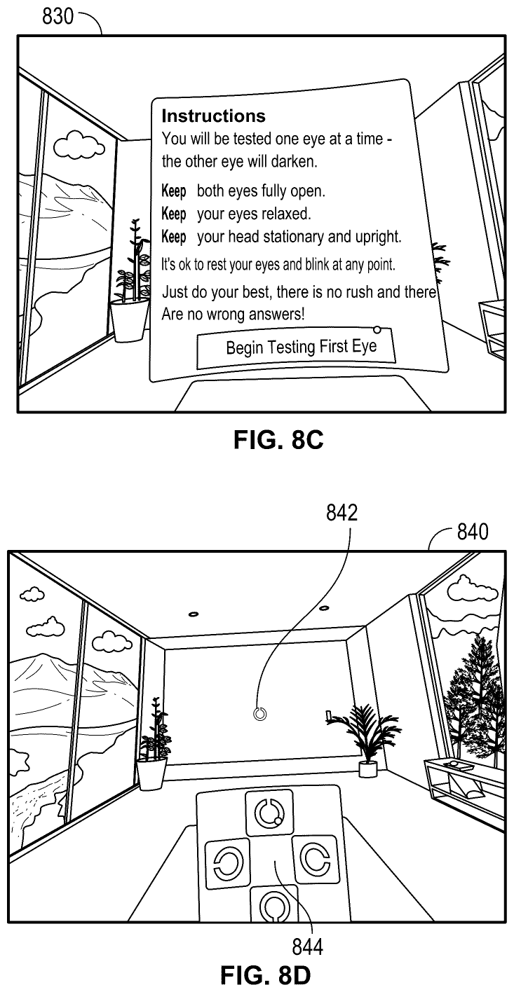

FIGS. 8A-8D include four diagrams of example graphical user interfaces 810, 820, 830, and 840 rendered to determine a visual acuity score in a virtual environment created by a headset device 140D, in accordance with some embodiments. The user interface 810 displays an information page including instructions on controlling a headset device 140D to select one of a plurality of optotype candidates to match a target optotype displayed in the virtual environment. The user interface 820 displays an information page including two optional ways of using the controller to select the one of the plurality of optotype candidates. The user interface 830 displays an information page including general guidelines on a visual acuity assessment process. The user interface 840 displays an optotype 842 that is projected on a screen that has a first distance L1 from a user's position in the virtual environment. In a second distance L2 near the user, a selection panel 844 including a plurality of optotype candidates is displayed, prompting the user to select one of the optotype candidates that matches the optotype 842. In some embodiments, in response to a user selection of the one of the optotype candidates, the optotype 842 displayed in the first distance L/is updated with a new optotype 842. Further, in some embodiments, the new optotype 842 spins at a fast rate for a shortened duration of time (e.g., 2 seconds), before it settles in place of the original optotype 842. In an example, the optotype 842 spins and gradually shrinks in size during the shortened duration of time.

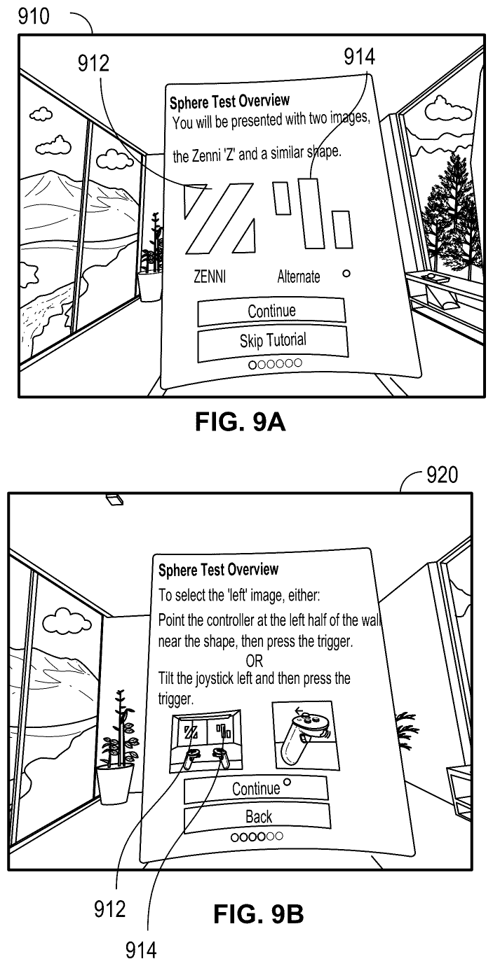

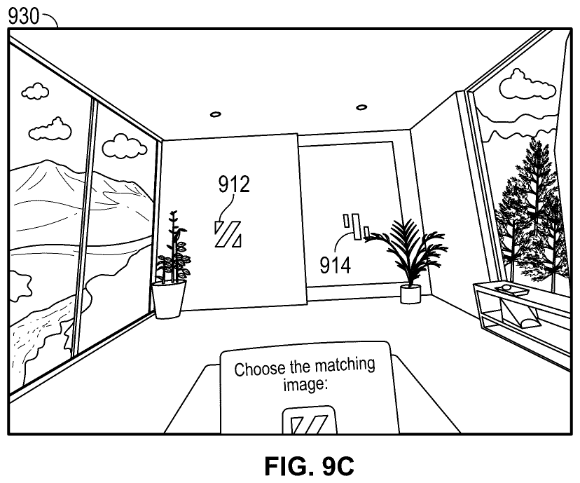

FIGS. 9A-9C include three diagrams of example graphical user interfaces 910, 920, and 930 rendered to determine a nearsighted or farsighted power in a virtual environment created by a headset device 140D, in accordance with some embodiments. The user interface 910 displays an information page explaining that two target optotypes 912 and 914 are displayed in the virtual environment. The user interface 920 displays an information page including two optional ways of using the controller to select one of the two target optotypes 912 and 914. The user interface 930 displays two target optotypes 912 and 914 that are projected on a screen that has a first distance L1 from a user's position in the virtual environment. In this example, the target optotype 912 located on the left is highlighted (e.g., by being displayed in a colored background). In a second distance L2 near the user, a confirmation panel 932 is displayed, prompting the user to select one of the two target optotypes 912 and 914. In some embodiments, in response to a user selection of the one of the two target optotypes 912 and 914, the two target optotypes 912 and 914 displayed in the first distance L1 is updated with a new pair of two target optotypes 912 and 914. Further, in some embodiments, each optotype 912 or 914 spins at a fast rate for a shortened duration of time (e.g., 2 seconds), before it settles in place of the original optotype 912 or 914. In an example, the optotype 912 or 914 spins and gradually shrinks in size during the shortened duration of time.

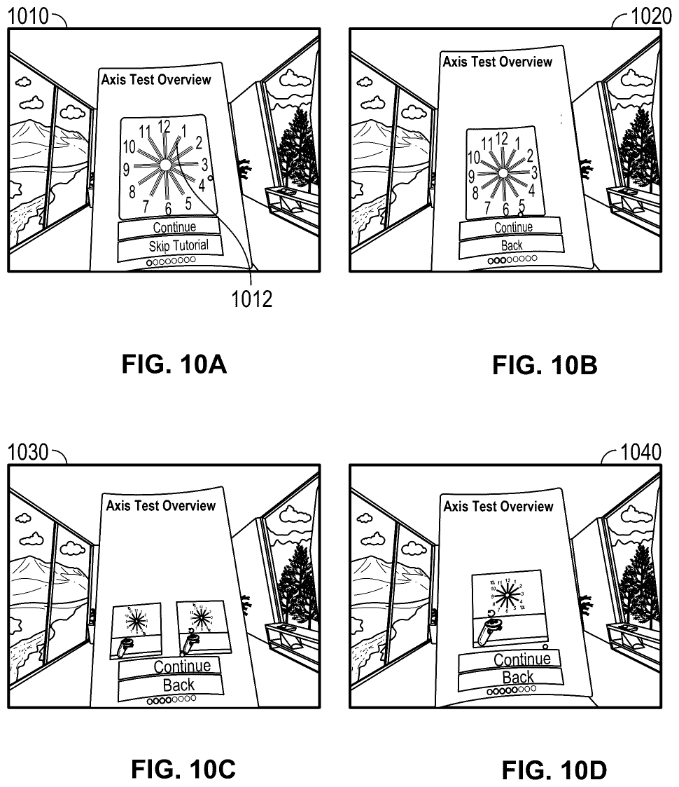

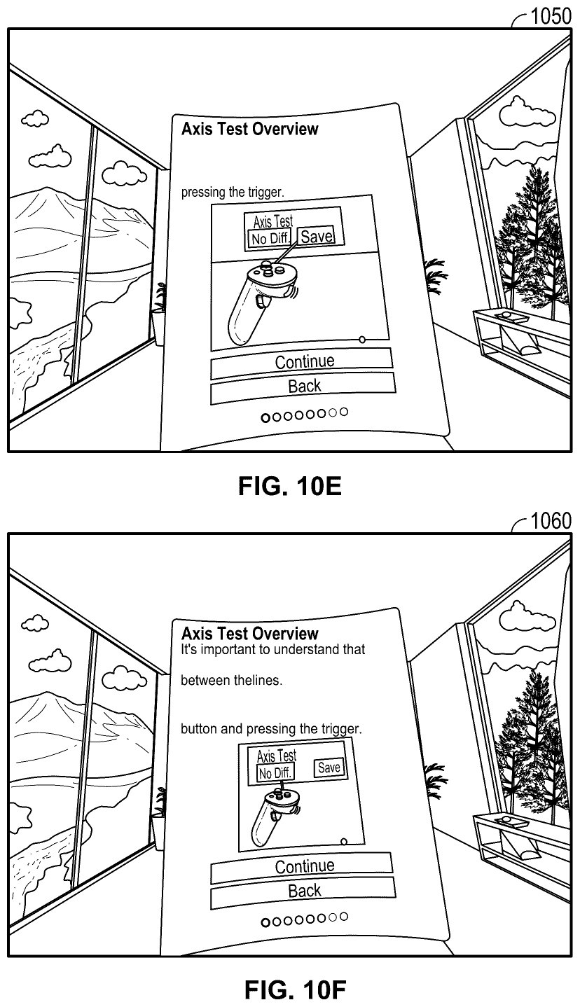

FIGS. 10A-10F include six diagrams of example graphical user interfaces 1010, 1020, 1030, 1040, 1050, and 1060 rendered to determine eye stigmatism in a virtual environment created by a headset device 140D, in accordance with some embodiments. The user interface 1010 displays an information page explaining that a clock diagram of converging numbered lines 1012 (which is a type of optotype) is displayed in the virtual environment. The user interface 1020 displays an information page explaining what is selected on the clock diagram of converging numbered lines 1012 displayed in the virtual environment. The user interface 1030 displays an information page including two optional ways of using the controller to select lines on the clock diagram of converging numbered lines 1012. The user interface 1040 displays an information page explaining a situation having equally clear lines on the clock diagram of converging numbered lines 1012. The user interface 1050 displays an information page including an instruction using the controller to submit a selection. The user interface 1060 displays an information page including an instruction using the controller to indicate that no difference is observed on the clock diagram of converging numbered lines 1012.

Some embodiments of a VR system are configured to enhance administration and experience of vision tests. The VR system includes a headset device 140D equipped with a display (sometimes referred to as a head-mounted display (HMD)). In some embodiments, the headset device 140D includes and one or more sensors for tracking one or more of eye movement, head orientation, and/or hand gestures of a user wearing the headset device 140D. In some embodiments, the headset device 140D is configured to execute a vision assessment application 328 configured to adaptively manage a sequence of vision tests based on the user's condition. In some embodiments, the headset device 140D is communicatively coupled to a server 102 configured to execute a server-side module for the vision assessment application 328, thereby managing the sequence of vision tests jointly with a device-side module of the vision assessment application 328 executed on the headset device. The vision assessment application 328 is configured to generate a virtual reality (VR) user interface corresponding to a three-dimensional (3D) virtual environment and render visual stimuli 338 in this 3D virtual environment. A range of different vision tests are conducted based on the visual stimuli within an immersive VR space.

In some embodiments, a headset device 140D includes one or more processors 302 and memory 306 storing instructions to execute the vision assessment application 328 for rendering visual stimuli 338 in an output device 312 (e.g., a display) and processing sensor data 342 collected from the sensors 360 in response to the visual stimuli 338. The sensor data 342 may be processed to determine vision test results 344 (e.g., eye movement patterns, response times, and visual perception accuracy) for the user. Further, in some embodiments, VR technology facilitates a personalized control scheme for navigating the vision tests. The personalized control scheme enables the user to interact with the test environment through intuitive hand gestures and eye movements, thereby providing a natural and engaging testing experience. The vision tests may be customized based on individual users' requirements and accommodate a wide range of vision impairments.

In some embodiments, the vision test results 344 are used to generate comprehensive reports on the user's visual performance. For example, the headset device 140D employs a deep learning model that correlates micro-expression data with vision test results 344 to provide holistic assessment of the user's ocular health. In some situations, the vision test results 344 are applied to identify vision conditions of the user and track changes of the vision conditions over time, thereby offering valuable insights to healthcare providers. In various embodiments of this application, eye images are captured and used to determine eye movement information automatically and without user intervention, which is an efficient solution to provide reliable supplemental information that cannot be provided by the user's active responses to visual stimuli.

### Example Vision Test System

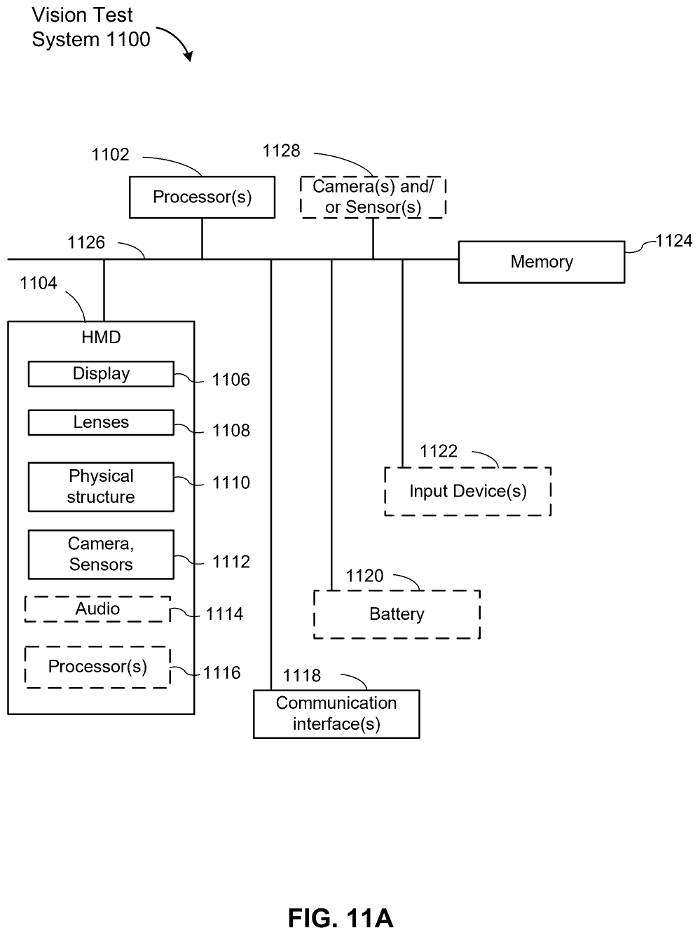

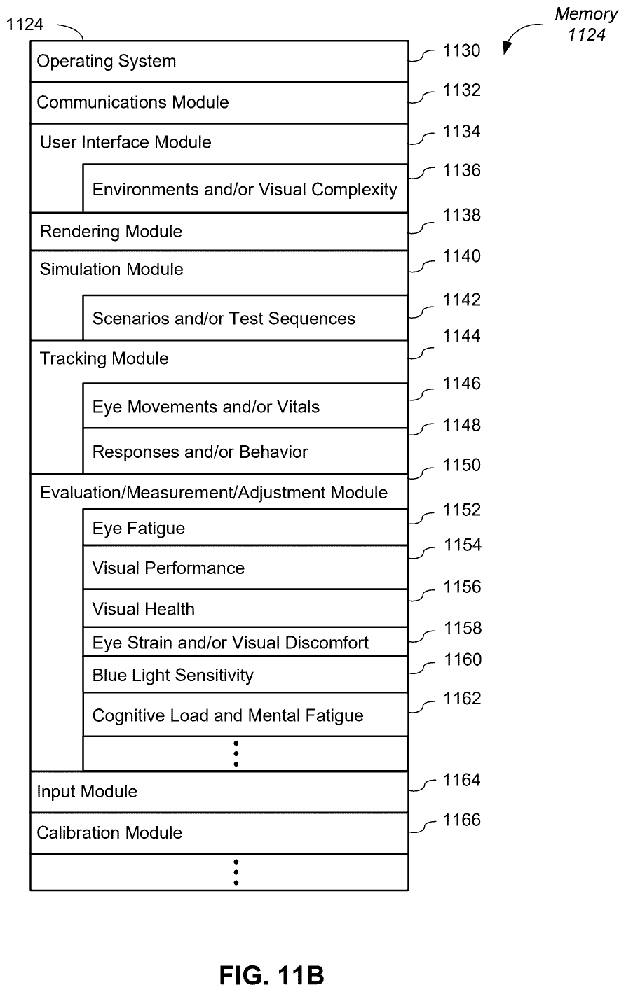

FIG. 11A is a diagram showing an example vision test system 1100, in accordance with some embodiments. The vision test system 1100 is implemented using a computer device (e.g., headset device 140D). The computer device includes one or more processors 1102, memory 1124 storing instructions to be implemented by the processor(s) 1102, a head-mounted display 1104, one or more network or other communications interfaces 1118, and one or more communication buses 1126 for interconnecting these and other optional components. The communication buses 1126 may include circuitry that interconnects and controls communications between system components.

The HMD 1104 may include a display 1106 (e.g., one or more high-resolution screens, one or more lenses 1108 (to focus and/or shape display images), cameras and/or sensors 1112 (e.g., outward camera 378, eye-tracking camera 366), and/or a physical structure 1110 (e.g., a structure that holds the components and configured to be worn on a head). The HMD 1104 optionally includes audio devices 1114 and one or more processors 1116 (instead of or in addition to the processors 1102, to implement instructions in the memory 1124). One or more cameras and/or sensors 1128 may be optionally included in some embodiments, instead of or in addition to the cameras and/or sensors 1112 integrated within the HMD 1104. The HMD may include, for example, high-resolution displays (e.g., 4K per eye), wide field of view (e.g., minimum 110 degrees), and/or adjustable interpupillary distance. The eye-tracking sensors can include, for example, high-precision infrared cameras, have a tracking frequency of 120 Hz or higher, have a latency of less than 5 milliseconds, and/or have an accuracy of sub-millimeter precision and/or 0.1 degrees in gaze direction.

In some embodiments, the computer device also includes one or more input devices 1122 (e.g., controllers and/or hand-tracking sensors). In some embodiments, the computer device also includes a battery 1120 (e.g., for standalone headsets). In some embodiments, the input device/mechanism 1122 includes a keyboard. In some embodiments, the input device/mechanism 1122 includes a “soft” keyboard, which is displayed as needed on the display 1106, for example, to enable a user to “press keys” that appear on the display 1106. In various embodiments, the communication interface(s) 1118 includes Wi-Fi, Bluetooth, and/or wired connections. In some embodiments, the input devices 1122 may include VR controllers and/or hand-tracking sensors. In some embodiments, the input devices 1122 may include one or more wearable devices for measuring, for example, intraocular pressure, tear film stability, and/or ocular blood flow.

In some embodiments, the memory 1124 includes high-speed random-access memory, such as DRAM, SRAM, DDR RAM, and/or other random-access solid state memory devices. In some embodiments, the memory 1124 includes non-volatile memory, such as one or more magnetic disk storage devices, optical disk storage devices, flash memory devices, or other non-volatile solid state storage devices. In some embodiments, the memory 1124 includes one or more storage devices remotely located from the processor(s) 1102. The memory 1124, or alternatively the non-volatile memory device(s) within the memory 1124, comprises a computer readable storage medium. Memory for headsets include, for example, Random-Access Memory (RAM), such as Low Power Double Data Rate RAM (LPDDR), used for running the operating system, applications, and/or handling real-time data processing. Memory 1124 may also include storage memory, such as flash memory, similar to smartphones (e.g., eMMC or UFS), for storing the operating system, applications, and/or user data. Video memory, often integrated with the GPU in mobile chipsets, can be used to handle graphics processing tasks. Cache memory, such as Static RAM (SRAM), can be used for high-speed memory used by the processors 1102 for quick data access.

Referring to FIG. 11B, in some embodiments, the memory 1124, or the computer readable storage medium of the memory 1124, stores the following programs, modules, and data structures, or a subset thereof.
an operating system 1130, which includes procedures for handling various basic system services and for performing hardware dependent tasks;
a communications module 1132, which is used for connecting the computing device to other computers and devices via the one or more communication network interfaces 1118 (wired or wireless) and/or via one or more communication networks, such as the Internet, other wide area networks, local area networks, metropolitan area networks, and so on;
a user interface module 1134 (sometimes referred to as the UI module 1134) for managing user interaction with VR/AR environments (sometimes referred to as three-dimensional virtual environments, photorealistic environments) and/or visual complexity 1136. The environments can include home environment, allowing users to launch apps, adjust settings, and/or navigate menus using virtual pointers or hand gestures. The environment can also include one or more visual scenarios. The UI module 1134 can include controls for initializing, modifying, and/or adapting visual complexity of the environment and/or visual scenarios. The UI may include interactive elements, such as menus, buttons, and control panels that are rendered in 3D space and can be interacted with using hand gestures or eye gaze;
a rendering module 1138 for handling the creation and/or display of 3D graphics in real-time. This can include a rendering pipeline, for example Unity's VR rendering pipeline, for optimizing frame rates and/or reducing latency for smooth VR/AR experiences;
a simulation module 1140 for creating and/or managing the rules, physics, and/or behaviors within the virtual environment. This can, for example, include PhysX in VR games, simulating realistic object interactions and gravity effects. The simulation module 1140 may include one or more scenarios and/or test sequences 1142;
a tracking module 1144 for processing sensor data to determine the position and orientation of the headset and/or controllers. The tracking module can track eye movements and/or vitals 1146, and/or responses and/or behavior 1148 (sometimes referred to as user responses), which may include, for example response times. In various embodiments, the eye movements 1146 includes, for example, gaze direction, fixation points, blink rate, squinting, and/or pupillary responses;
an evaluation and/or measurement module 1150 for analyzing tracked data, user interactions and/or system performance for optimization and/or adaptation and feedback to determine and/or measure, for example, eye fatigue 1152, visual performance 1154, visual health 1156, eye strain and/or visual discomfort 1158, blue light sensitivity 1160, and/or cognitive load and/or mental fatigue 1162. In some embodiments, the evaluation module performs real-time data processing and/or analysis, calculates performance metrics (e.g., reaction times, error rates), and/or assesses color perception and/or wavelength sensitivity. In some embodiments, the module 1150 can include one or more recommendation engines for AI-driven analysis for personalized recommendations, and/or suggestions. In some embodiments, the module 1150 also includes a reporting system for report generation, visual field mapping and/or color sensitivity profiling;
an input module 1164 for interpreting and/or processing user input from various sources (e.g., controllers, hand tracking, voice commands). This module can include hand tracking software, translating hand and finger movements into VR interactions; and/or
a calibration module 1166 for alignment of virtual and physical elements, often including initial setup procedures, for calibrating the device and/or experimental setups based on user data, which can include setup, and/or guiding users through the process of defining their viewing and/or test area and/or calibrating controllers.

The UI module 1134 may generate interactive visual elements that allow users to navigate and interact with the highly realistic 3D virtual world. This may include creating menus and buttons that appear to exist within a 3D space, implementing gesture-based controls that feel natural in the virtual world, designing visual feedback that matches the aesthetic of the environment, and/or integrating information displays seamlessly with the surroundings. The UI module 1134 may utilize various implementation methods, such as game engines (e.g., Unity, Unreal Engine) for UI implementation and integration, and/or 3D modeling software for creating UI assets.

The processing may include processing on host computers for tethered VR headsets, may include on-device processing for standalone VR/AR headsets, and/or cloud processing for computationally intensive tasks. In various embodiments, the UI module 1134 enhances user immersion and presence by, for example, creating UI elements that look and feel like they belong in the photorealistic environment, implementing holographic displays or interactive physical objects, and/or supporting interaction through VR controllers or hand tracking. In some embodiments, the UI module 1134 adapts the UI to different types of virtual environments, ensuring consistency and usability across various scenarios. In some embodiments, the UI module 1134 also handles user input (e.g., in collaboration with an input module, described below) through multiple modalities, including hand tracking, eye tracking, and controller input, to facilitate seamless interaction with the generated UI.

In some embodiments, the rendering module 1138 integrates the VR user interface elements with the photorealistic environment, ensuring proper depth, occlusion, and lighting interactions. In some embodiments, the rendering module 1138 implements stereo rendering techniques to create a sense of depth and dimensionality for the UI elements when displayed on the HMD. In some embodiments, the rendering module 1138 applies distortion correction and lens-specific optimizations to ensure the UI is properly displayed on the HMD's optics. In some embodiments, the rendering module 1138 utilizes techniques like foveated rendering to optimize UI rendering performance, particularly for resource-intensive photorealistic environments. In some embodiments, the rendering module 1138 handles dynamic UI updates and animations in real-time, maintaining consistent frame rates crucial for comfortable VR experiences. In some embodiments, the rendering module 1138 implements anti-aliasing and other image quality enhancements specific to HMD displays to ensure crisp, readable UI elements.

In various embodiments, the one or more scenarios 1142 can include real-world scenarios, dynamic real-world visual experiences, test sequences with progressively finer details, real-world motion and target recognition visual tasks, and/or various visual scenarios (including, for example, scenarios with different lighting conditions). In some embodiments, the simulation module 1140 may be further configured to generate and manage real-world scenarios in the VR user interface, such as simulating everyday activities or specific professional environments. In some embodiments, the simulation module 1140 may be further configured to create and control testing sequences that progressively introduce finer details and objects at varying depths within the three-dimensional virtual environment, allowing for comprehensive visual acuity assessment.

In some embodiments, the simulation module 1140 may be further configured to simulate dynamic real-world visual experiences by incorporating moving objects, changing environments, and interactive elements that respond to user actions. In some embodiments, the simulation module 1140 may be further configured to implement real-world motion and target recognition tasks, such as tracking moving objects or identifying specific targets within complex visual scenes. In some embodiments, the simulation module 1140 may be further configured to generate visual scenarios that require focus adjustments, simulating the need to shift focus between near and far objects in the virtual environment.

In some embodiments, the simulation module 1140 may be further configured to create a diverse range of visual scenarios, each designed to test different aspects of vision or simulate specific real-world conditions. In some embodiments, the simulation module 1140 may be further configured to implement lighting simulation algorithms to create visual scenarios with varying lighting conditions, including daylight, twilight, indoor lighting, and challenging low-light situations. In some embodiments, the simulation module 1140 may be further configured to utilize the PhysX engine or similar physics simulation tools to ensure realistic object behavior and interactions within these scenarios, enhancing the authenticity of the simulated experiences.

In some embodiments, the simulation module 1140 may be further configured to integrate with the rendering module 1138 to ensure that simulated scenarios are accurately displayed on the HMD, maintaining the intended visual fidelity and realism. In some embodiments, the simulation module 1140 may be further configured to allow customization and parametric control of scenarios, enabling the creation of tailored visual experiences for specific testing or training purposes.

For eye testing purposes, some embodiments track eye movements and response times with high frequency and precision. In some embodiments, for eye movements, and specifically for saccades, rapid movements of the eye between fixation points are tracked at rates of at least 100-500 Hz. This high frequency helps capture the quick and brief nature of these movements accurately. For fixations, periods where the eyes are relatively stationary and focused on a single point are tracked at slightly lower rates, but typically in the range of 50-100 Hz, to ensure precise measurement of duration and stability. For smooth pursuit (e.g., movements where the eyes smoothly follow a moving object), eye movements are also tracked at high rates (100-200 Hz) to accurately capture the speed and trajectory of the eye movements.

In some embodiments, the high-precision eye tracking is achieved through a combination of hardware and software algorithms. For example, the hardware may include multiple infrared cameras strategically positioned around each eye, capturing images at a minimum of 1,000 frames per second. These cameras may use custom-designed sensors with a minimum resolution (e.g., at least 5 megapixels) for detailed capture of eye movements. The software may use computer vision algorithms, including, for example, convolutional neural networks (CNNs), for pupil detection and/or corneal reflection tracking. These algorithms may process the high-frame-rate imagery in real-time, employing, for example, parallel computing techniques to maintain low latency. Some embodiments use a predictive model to anticipate eye movements, further reducing effective latency. Calibration routines, for example, may employ active learning methods to rapidly adapt to individual eye physiologies. Using such a combination of high-speed imagery, advanced image processing, and/or predictive modeling some embodiments can track eye movements with sub-millimeter precision, a latency of less than 5 milliseconds, and/or an operational frequency exceeding 120 Hz.

In some embodiments, for response times, specifically for reaction time (e.g., the time it takes for a person to respond to a visual stimulus, such as pressing a button when a light appears), are tracked with millisecond accuracy. This typically means using sampling rates of 1000 Hz or higher to ensure precise measurement. For decision time, which may include, for example, the duration between recognizing a visual stimulus and making a decision based on, are tracked using high-frequency tracking, typically around 500-1000 Hz, to accurately capture the cognitive processing speed.

High-frequency tracking ensures that no significant movement or response detail is missed, providing a more accurate and reliable assessment of visual function. Real-world visual tasks involve rapid and complex eye movements, and high-frequency tracking allows for a more detailed analysis of how well the eyes can handle such tasks. Subtle abnormalities in eye movements or delays in response times can be early indicators of visual or neurological problems. High-frequency tracking helps in detecting these issues at an early stage. In some embodiments, for eye testing, continuous tracking of eye movements and response times is performed at high frequencies (e.g., ranging from 50 Hz to 1000 Hz) to ensure precise and comprehensive data collection. While both eye testing and VR games benefit from eye-tracking technology, the former requires much higher precision, frequency, and reliability for clinical and diagnostic purposes. In contrast, VR games prioritize user experience and real-time interaction, allowing for lower precision and frequency in tracking (e.g., 30-120 Hz).

In some embodiments, the tracking module 1144 may be further configured to continuously track eye movements and response times to visual stimuli presented in the one or more real-world scenarios simulated in the VR user interface, using the camera at high frequencies (e.g., 100-500 Hz for saccades, 50-100 Hz for fixations). In some embodiments, the tracking module 1144 may be further configured to track eye movements and response times to visual stimuli presented in the testing sequence, capturing data throughout the progression of finer details and varying depths in the three-dimensional virtual environment. In some embodiments, the tracking module 1144 may be further configured to monitor eye movements and response times to visual stimuli presented in the dynamic real-world visual experience, adapting to changing environmental conditions and moving objects within the simulation.

In some embodiments, the tracking module 1144 may be further configured to track eye movements and response times specifically for real-world motion and target recognition visual tasks, providing detailed data on how users visually engage with moving objects and identify targets in complex scenes. In some embodiments, the tracking module 1144 may be further configured to monitor dynamic focus adjustments in response to visual stimuli presented in various visual scenarios, capturing data on how quickly and accurately users can shift focus between near and far objects in the virtual environment.

In some embodiments, the tracking module 1144 may be further configured to track user interactions and responses to visual stimuli across a range of visual scenarios, including those with different lighting conditions, providing comprehensive data on visual performance under various environmental conditions. In some embodiments, the tracking module 1144 may be further configured to integrate with the simulation module 1140 to ensure synchronized tracking of eye movements and responses with the presented visual stimuli across all types of simulated scenarios.

In some embodiments, the tracking module 1144 may be further configured to process and/or analyze the collected high-frequency data in real-time, providing immediate feedback on visual performance and enabling dynamic adjustments to the testing or training protocols as needed. These enhanced tracking capabilities ensure that the system can capture detailed, precise data on eye movements and responses across a wide range of simulated scenarios, supporting comprehensive analysis of visual function and performance in virtual reality environments. In some embodiments, the tracking module 1144 may be further configured to continuously track eye movements and response times in response to visual stimuli presented in the one or more dynamic lighting scenarios. This tracking is performed using the camera at high frequencies (e.g., 100-500 Hz for saccades, 50-100 Hz for fixations) to capture rapid eye movements in changing light conditions.

In some embodiments, the tracking module 1144 may be further configured to continuously monitor and record pupil data, including pupil dilation and constriction, in response to visual stimuli presented in the one or more dynamic lighting scenarios. This pupil tracking is performed at high frequencies (e.g., 120-250 Hz) to capture subtle and rapid changes in pupil size as lighting conditions change. In some embodiments, the tracking module 1144 may be further configured to specifically track eye movements, including saccades, fixations, and smooth pursuit, in response to visual stimuli presented in the one or more dynamic lighting scenarios. This tracking captures how the eyes adapt and respond to changing light levels, moving shadows, or shifting light sources within the virtual environment.

In some embodiments, the tracking module 1144 may be further configured to synchronize the eye tracking data with the simulated lighting conditions, allowing for precise analysis of how different lighting scenarios affect eye movements, pupil reactions, and response times. In some embodiments, the tracking module 1144 may be further configured to process and analyze the collected high-frequency eye movement, pupil, and response time data in real-time, providing immediate feedback on visual performance under varying lighting conditions.

In some embodiments, the tracking module 1144 may be further configured to integrate with the simulation module 1140 to ensure that eye tracking is precisely coordinated with the dynamic changes in lighting conditions, allowing for accurate assessment of visual adaptation to light changes. These enhancements enable the system to capture detailed, time-synced data on eye movements, pupil reactions, and/or response times, specifically in relation to changing lighting conditions in the virtual environment, supporting comprehensive analysis of visual function and/or performance under various lighting scenarios.

In some embodiments, the evaluation and/or measurement module 1150 may be further configured to analyze eye movements and response times captured by the tracking module 1144 to evaluate visual acuity and perception. This may include, for example, assessing the accuracy and speed of eye movements in response to stimuli of varying sizes and contrasts. In some embodiments, the evaluation and/or measurement module 1150 may be further configured to utilize eye movement data and response times to specifically test and evaluate visual acuity, considering factors such as the minimum resolvable detail and reaction speed to visual stimuli.

In some embodiments, the evaluation and/or measurement module 1150 may be further configured to assess depth perception, motion detection, and spatial awareness by analyzing eye movements and response times during tasks that involve tracking moving objects, judging distances, and navigating 3D environments. In some embodiments, the evaluation and/or measurement module 1150 may be further configured to measure dynamic visual acuity by evaluating eye movements and response times when tracking moving targets of varying speeds and sizes, quantifying the ability to discern details of objects in motion. In some embodiments, the evaluation and/or measurement module 1150 may be further configured to analyze dynamic focus adjustment data to measure astigmatism, examining how the eyes focus on lines and shapes at different orientations and distances.

In some embodiments, the evaluation and/or measurement module 1150 may be further configured to process user interactions and responses to visual stimuli to measure and adjust for visual distortions. This may include, for example, analyzing how users perceive and interact with potentially distorted images or environments in the VR interface. In some embodiments, the evaluation and/or measurement module 1150 may be further configured to evaluate user interactions and responses in low-light scenarios to measure night blindness, assessing visual performance and adaptation in simulated nighttime or dim lighting conditions. In some embodiments, the evaluation and/or measurement module 1150 may be further configured to integrate with the simulation module 1140 to ensure that evaluations and measurements are precisely correlated with the specific visual stimuli and environmental conditions presented in each test scenario.

In some embodiments, the evaluation and/or measurement module 1150 may be further configured to implement advanced algorithms to interpret complex eye movement patterns and response data, translating raw tracking data into meaningful metrics for each visual function being assessed. In some embodiments, the evaluation and/or measurement module 1150 may be further configured to generate comprehensive reports detailing the results of visual function assessments, including quantitative measures of visual acuity, depth perception, motion detection, astigmatism, and night vision capabilities. In some embodiments, the evaluation and/or measurement module 1150 may be further configured to provide real-time feedback during testing sessions, allowing for dynamic adjustment of test parameters based on ongoing performance and response patterns. These features enable the system to conduct thorough, quantitative evaluations of various aspects of visual function based on eye movement data and/or user responses, supporting detailed analysis and measurement of visual capabilities within the VR environment.

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise rearranged in various embodiments. In some embodiments, the memory 1124 stores a subset of the modules and data structures identified above. Furthermore, in some embodiments, the memory 1124 stores additional modules or data structures not described above. Example details and/or operations of the modules, data structures, applications and/or procedures, are further described below, according to some embodiments.

Although FIG. 11A shows a computing device, FIG. 11A is intended more as a functional description of the various features that may be present rather than as a structural schematic of the embodiments described herein. In practice, and as recognized by those of ordinary skill in the art, items shown separately could be combined and some items could be separated.

### VR Eye Fatigue Monitoring and Adjustment System

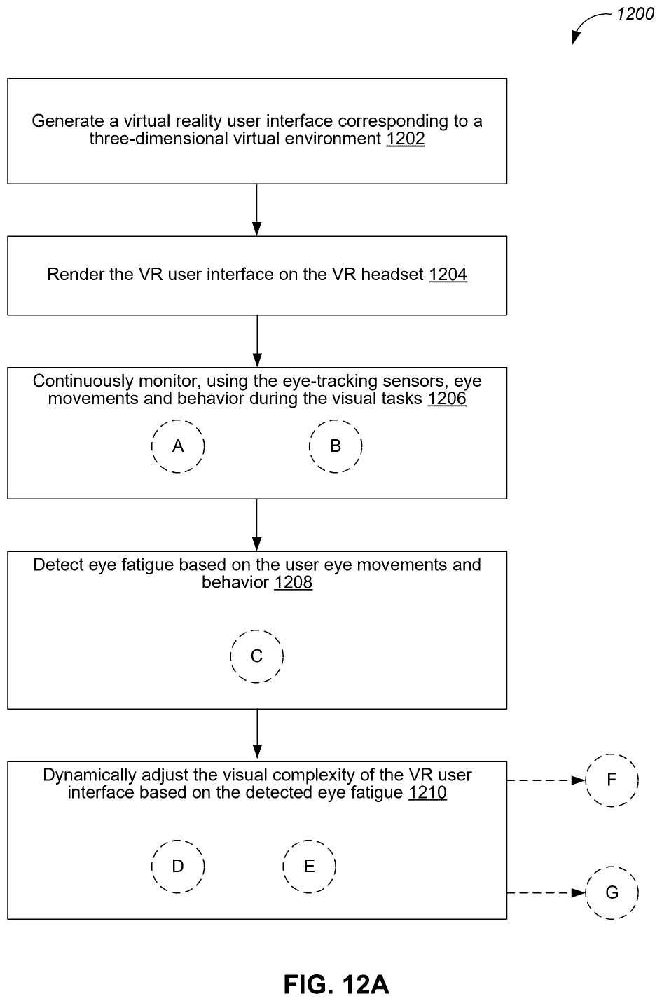

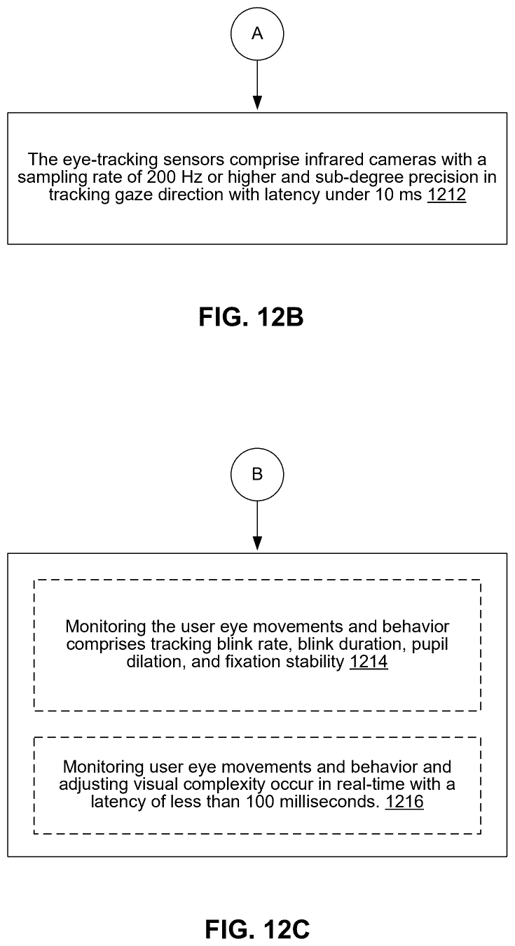

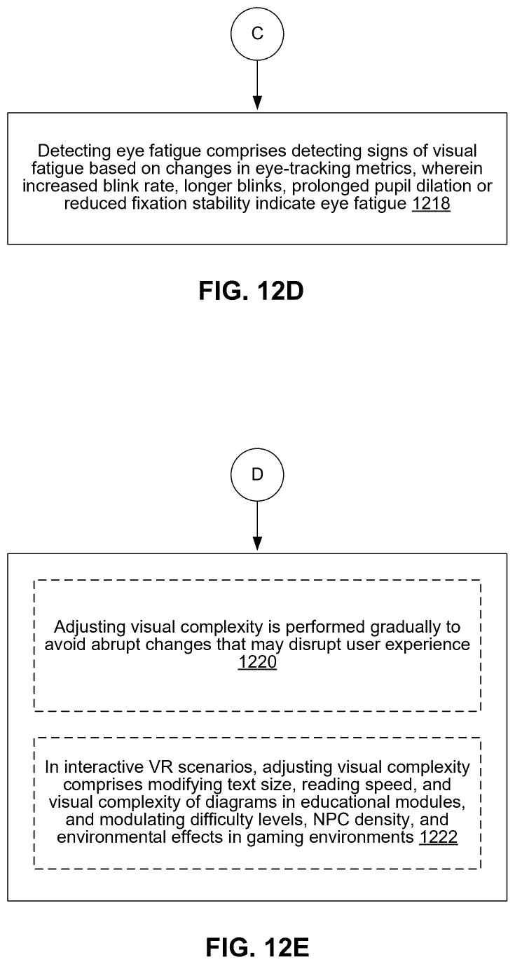

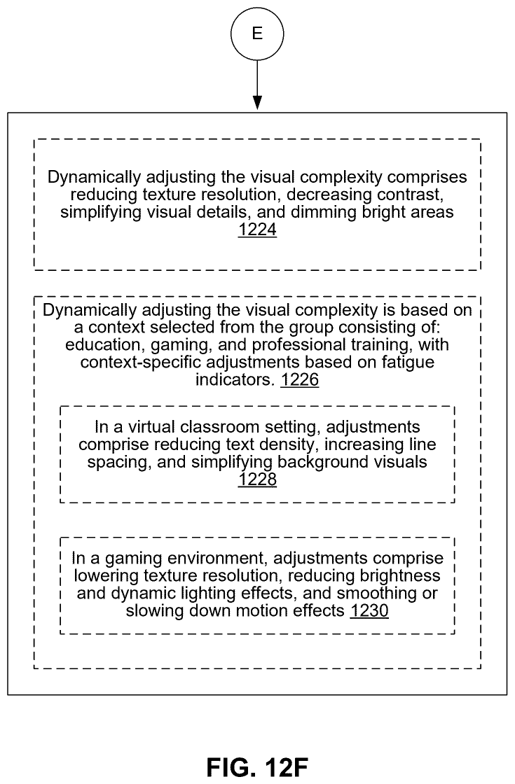

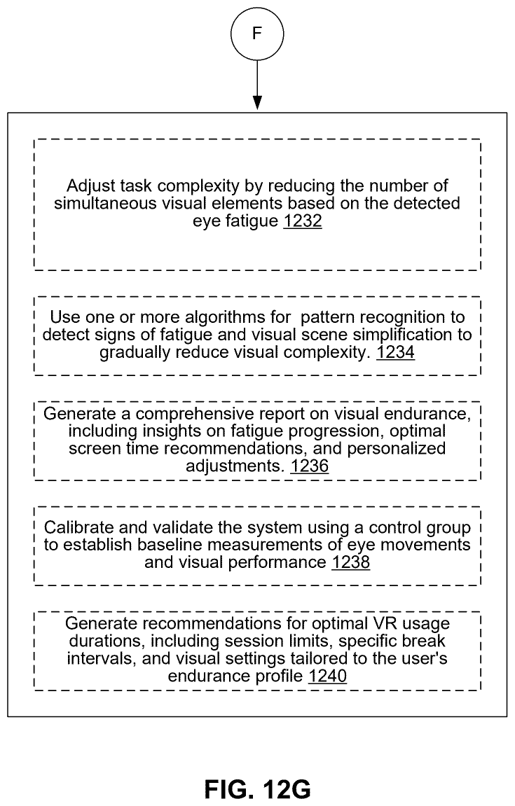

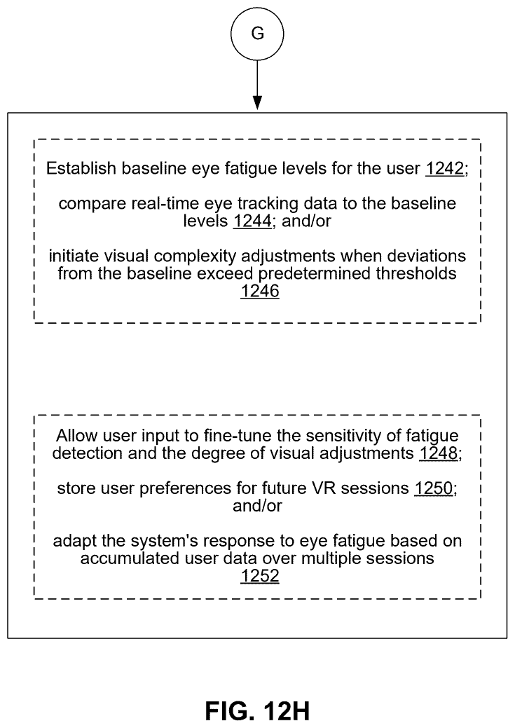

According to some embodiments, the vision test system 1100 described above is configured to implement a virtual reality (VR) system that adjusts visual complexity based on real-time eye fatigue monitoring. FIGS. 12A-12H show a flow diagram of an example process 1200 for implementing a virtual reality (VR) system that adjusts visual complexity based on real-time eye fatigue monitoring, according to some embodiments. Conventional methods of eye fatigue monitoring often involve subjective questionnaires and manual observation, which are prone to inaccuracies and do not provide real-time feedback. These methods lack the ability to dynamically adjust visual content based on user fatigue levels, making them less effective in preventing eye strain during prolonged use of digital devices. Accordingly, the techniques described herein address at least some of these problems.

The computer device 140 (e.g., the computing device described above in reference to FIGS. 11A and 11B) generates (e.g., in step 1202) (e.g., using the UI module 1134) a virtual reality (VR) user interface (UI) corresponding to a three-dimensional virtual environment (e.g., an environment 1136). In some embodiments, game engines (e.g., platforms like Unity or Unreal Engine) are used to implement the UI and integrate it with the virtual environment. 3D modeling software can be used for creating assets that may be part of the UI in a photorealistic environment.

In some embodiments, this step is performed on a host computer, whereby the main processing unit (CPU) and graphics card (GPU) of the computer connected to a VR/AR headset handles much of the heavy lifting for generating and rendering the UI. This can be useful for tethered VR headsets that rely on a powerful PC for processing. In some embodiments, this step is performed on the headset itself. Standalone VR/AR headsets have onboard processors that can handle some or all of the UI generation and rendering. This on-device processing provides responsive, low-latency interactions. Cloud processing can also be used for some aspects of UI generation. For example, tasks requiring heavy computation might be offloaded to cloud servers and streamed to the headset. A combination of the above, with some elements pre-baked during development, some processed on a host PC, and some handled by the headset itself, can be used in some embodiments.

In some embodiments, the step of generating a VR UI corresponding to a photorealistic environment includes creating interactive visual elements that allow users to navigate and interact with a highly realistic 3D virtual world. Photorealistic virtual environment refers to a 3D digital space that looks and behaves as close to reality as possible. Advanced graphics, lighting, textures, and/or physics simulations can be used to create a highly detailed and lifelike virtual world. VR user interface is the set of visual elements, controls, and/or interaction methods that allow users to navigate, manipulate, and/or engage with the virtual environment. In VR, these interfaces are designed to be intuitive and immersive, often blending seamlessly with the virtual world.

Generating the interface may include generating UI elements that are both functional and visually consistent with the photorealistic environment. In various embodiments, this includes menus and buttons that appear to exist within the 3D space, gesture-based controls that feel natural in the virtual world, visual feedback that matches the aesthetic of the environment, and/or information displays that integrate with the surroundings. The computer device 140 creates an interface that enhances the user's sense of presence and immersion in the virtual world. This often means making UI elements that look and feel like they belong in the photorealistic environment, such as holographic displays or physical objects that the user can interact with using VR controllers or hand tracking.

Eye testing using photorealistic environments offers several advantages compared to traditional methods. Photorealistic environments provide a more accurate and comprehensive assessment of visual function. For example, photorealistic environments provide realistic simulation, mimic real-world conditions much more accurately than traditional eye charts or simple visual tests. This allows for a more accurate assessment of how well a person can see in everyday situations. These environments can change dynamically to simulate different lighting conditions, distances, and angles, providing a more comprehensive test of visual capabilities, including peripheral vision and depth perception.

Patients, especially children or those with attention difficulties, may find photorealistic environments more engaging than standard tests, leading to more reliable results as they are more likely to fully participate in the testing process. Traditional eye tests often focus on static images and high-contrast letters. Photorealistic environments, on the other hand, can be used to present complex, real-world visual tasks that can better assess functions like motion detection, contrast sensitivity, and/or color perception. Furthermore, the photorealistic environment can be customized to the specific needs or conditions of the patient, such as simulating the individual's workplace or home setting, providing a personalized and relevant assessment of their vision.

More complex and varied testing scenarios, which photorealistic environments can help simulate, can help in the early detection of visual problems that might not be apparent in traditional tests. This includes issues related to glare, night vision, and visual processing speeds. Advanced eye-tracking technology, specific examples of which are described herein, can be used in photorealistic environments to provide objective data on eye movements, fixation points, and response times, offering a more detailed analysis of visual function. For patients undergoing vision therapy or rehabilitation, photorealistic environments can provide a controlled yet realistic setting for practicing visual skills, making the training more effective and directly applicable to real-world tasks. Overall, eye testing using photorealistic environments described herein, represents a significant advancement in optometry and vision science, offering a richer, more detailed, and accurate assessment of visual health.

The computer device 140 renders (e.g., in step 1204) (e.g., using the rendering module 1138) the VR user interface on the VR headset (e.g., on the HMD 1102). In some embodiments, photorealistic environments are displayed by leveraging various techniques and technologies described herein, according to some embodiments. Some embodiments use photogrammetry to create highly detailed 3D models from a set of photographs. By capturing real-world objects or environments from multiple angles, photogrammetry helps reconstruct their geometry and computer textures with a high degree of realism. In some embodiments, these models are then imported into the VR environment (sometimes referred to as the photorealistic environment or three-dimensional virtual environment).

Some embodiments provide 360-degree photography and videography. In some embodiments, VR devices display panoramic 360-degree photos and videos, which provide an immersive and photorealistic representation of real-world environments. In some embodiments, these are captured using specialized camera rigs or stitched together from multiple camera feeds. Some embodiments use real-time ray tracing. Modern graphics hardware and rendering techniques like real-time ray tracing help simulate the behavior of light in a physically accurate manner. By accurately modeling the interaction of light with materials, surfaces, and objects, ray tracing produces highly photorealistic images and environments in real-time. Some embodiments provide high-resolution textures and models. VR devices leverage high-resolution textures and detailed 3D models to create environments that closely resemble reality.

Some embodiments generate photorealistic environments using a combination of advanced rendering techniques and real-world data integration. High-resolution textures, captured through photogrammetry, may be mapped onto geometrically accurate 3D models. Global illumination algorithms, including ray tracing and radiosity, may be employed to simulate realistic lighting conditions. Physical-based rendering (PBR) materials may be used to accurately represent surface properties, such as reflectivity, roughness, and subsurface scattering. Dynamic elements, such as moving objects or changing weather conditions, may be simulated using particle systems and fluid dynamics algorithms. Some embodiments also incorporate real-time occlusion culling and level-of-detail (LOD) management to maintain high frame rates while preserving visual fidelity. To ensure consistency and repeatability, each photorealistic environment may be generated based on predefined parameters. These parameters may include lighting conditions, object placements, and/or atmospheric effects. In this way, some embodiments create controlled yet highly detailed environments that can be easily replicated or modified for different testing scenarios.

In some embodiments, the environments are created using techniques like photogrammetry, 3D scanning, or manually by artists and designers. Some embodiments use physically based rendering (PBR). PBR includes simulating the behavior of materials and their interactions with light based on real-world physics principles. By accurately modeling materials and their properties, such as roughness, metallic properties, and reflectance, PBR produces highly realistic visuals in VR environments. Some embodiments use image-based rendering, which includes using real-world photographs or video footage as the basis for rendering virtual environments. In some embodiments, by projecting and blending these images onto 3D geometry, a highly photorealistic environment is created. In some embodiments, VR devices capture real-world lighting information using techniques like light probes or environmental capture. This data can then be used to accurately simulate and recreate realistic lighting conditions within the virtual environment. By combining the techniques described herein and leveraging the latest advancements in graphics hardware and rendering algorithms, VR devices can provide highly immersive and photorealistic virtual experiences that closely resemble real-world environments.

Photorealistic environments used for eye testing can differ significantly from those used in VR games in several aspects, including design, functionality, and application. Photorealistic environments for eye testing are designed for precision, control, and repeatability to assess visual functions accurately, while those for VR games focus on creating immersive, interactive, and enjoyable experiences for entertainment. In contrast to VR games, eye testing requires clinical precision. Accordingly, some embodiments provide highly controlled and repeatable conditions for accurate diagnosis and assessment of visual functions. In some embodiments, specific scenarios are tailored to simulate real-world conditions that are relevant for visual testing, such as different lighting conditions, contrast levels, and visual tasks like reading or recognizing objects. Environments are kept consistent across tests to ensure reliable results. This includes controlled variations in visual stimuli to test specific aspects of vision.

Eye testing also requires precision tracking. Accordingly, some embodiments utilize high-precision eye-tracking to measure fine details of eye movements, fixations, and/or response times. Some embodiments collect accurate data for clinical analysis, including metrics, such as saccadic latency, fixation stability, and smooth pursuit accuracy. Some embodiments can include standardized visual tests, such as visual acuity tests, contrast sensitivity tests, and visual field tests. Example headsets that may be used for implementing the system and/or methods described herein include Varjo VR-3, which integrates high-resolution displays (over 70 PPD) with eye-tracking technology that captures eye movements at 200 Hz. These headsets are particularly suitable for applications requiring precise eye-tracking to adjust visual complexity in real-time.

In some embodiments, the photorealistic virtual environment prioritizes precision, control, repeatability and/or data collection over immersion, interaction, variety and/or user experience to assess visual functions accurately.

For example, a photorealistic environment for eye testing that includes a simulated driving environment can include a controlled simulation of driving conditions at night or in fog, designed to assess visual acuity, peripheral vision, and reaction times. The environment would include standardized visual stimuli, such as road signs, other vehicles, and pedestrians, which appear in predetermined patterns and intervals. For repeatability, each test is consistent, with the same conditions and stimuli presented in the same manner each time. This ensures that results can be reliably compared across different sessions or subjects. As another example, a photorealistic environment for eye testing that includes reading and office tasks can include a photorealistic simulation of an office environment with various reading tasks. This could include reading text on a computer screen, paper documents, and recognizing icons or objects on a cluttered desk.

For repeatability, text size, font, contrast, and lighting conditions are kept constant across tests. This allows precise measurement of reading speed, accuracy, and visual fatigue under standardized conditions. As yet another example, a supermarket simulation can include a virtual supermarket where patients are asked to locate and identify products on shelves. The environment would include standardized lighting, product placement, and visual clutter. For repeatability, the position and appearance of products remain the same in each test, ensuring that any changes in performance are due to the patient's vision and not variations in the environment. Eye testing environments prioritize controlled and repeatable conditions to ensure accurate measurement of visual functions instead of, or in addition to, focusing on creating immersive and interactive experiences that engage and entertain players. Eye testing environments are standardized to eliminate variables that could affect the results. A goal of eye testing environments, such as the ones described herein, is to collect precise data for clinical analysis, more than merely providing enjoyable user experience.

In the context of a photorealistic virtual environment designed for precise visual function assessment, qualities, such as precision, control, repeatability, and data collection, may be quantified or measured using the following methodologies. Precision may be quantified by measuring the variance in visual acuity scores or reaction times when the same stimuli are presented multiple times under identical conditions. A lower variance would indicate higher precision. Additionally, the spatial resolution of the visual stimuli may be quantified by the pixel density in the VR environment, where higher pixel density corresponds to higher precision in visual representation.

Control may be measured by assessing the fidelity of the virtual environment to real-world parameters. For instance, in a simulated driving environment, control may be quantified by how accurately the speed, direction, and lighting conditions match predefined standards. Metrics, such as frame rate stability, latency in rendering, and synchronization with real-world physics (e.g., gravity, friction) may serve as quantitative measures of control.

Repeatability may be quantified by the consistency of test results across multiple sessions. Statistical methods, such as calculating the intraclass correlation coefficient (ICC), may be used to measure the reliability of visual function assessments over time. A high ICC value may indicate that the VR environment consistently produces similar outcomes, highlighting strong repeatability. The effectiveness of data collection may be measured by the amount and quality of data points gathered during each session. This may include the resolution of eye-tracking data, the accuracy of response time measurements, and the granularity of physiological data (e.g., pupil dilation, heart rate). The completeness of data collection, indicated by minimal data loss or artifacts, may also be used.

In some embodiments, the photorealistic virtual environment corresponds to an environment selected from the group consisting of: urban streets, natural landscapes, indoor settings (e.g., living rooms, offices), and crowded public spaces (e.g., malls, transportation hubs). The system may define, store, and/or use scenarios with a level of detail and movement similar to busy intersections or trails by leveraging advanced computer graphics techniques and/or a robust database architecture.

For example, each environment, such as a busy intersection or a forest trail, may be defined by its unique set of visual and interactive elements. For a busy intersection, the system may include parameters, such as traffic density, pedestrian flow, vehicle speeds, traffic light cycles, and/or ambient noise levels. For a forest trail, the environment may include varying terrain textures, dynamic lighting based on time of day, and/or movement of flora and fauna.

Optionally, scenarios may be stored as modular data sets within the system's database. Each scenario may include 3D models, textures, lighting maps, and/or behavioral scripts that dictate how objects in the environment interact with the user.

For example, a busy intersection scenario may store detailed vehicle models, pedestrian avatars, and/or algorithms controlling their movement patterns. The storage system may be optimized for quick retrieval and modification, allowing scenarios to be adapted based on user requirements or testing protocols. The system may use these scenarios by dynamically loading them into the VR environment during testing.

The criteria for what constitute each environment can include various factors. For example, the criteria can include a Level of Detail (LOD). For busy intersections, for example, the LOD may include high-resolution textures for vehicles, road surfaces, and buildings, alongside complex shadowing and/or reflection effects. For trails, for example, the LOD may emphasize realistic foliage, ground textures, and/or subtle environmental movements like wind in the trees. The criteria can also include a movement complexity. In busy intersections, movement complexity may involve multiple objects (e.g., vehicles, pedestrians) moving at varying speeds and/or trajectories.

For trails, movement complexity may include the swaying of trees, shifting light through the canopy, and/or the user's interaction with uneven terrain; (iii) interactivity: The degree to which the user can interact with the environment may also define its complexity. In an intersection, users may respond to traffic signals, navigate around obstacles, and/or follow a vehicle's trajectory. In a trail scenario, interaction may include avoiding obstacles, tracking wildlife, and/or responding to changes in terrain.

In some embodiments, the VR user interface allows a user to navigate through virtual environments using natural head and eye movements, mimicking real-world interactions and responses. Natural head and eye movements in the context of a VR environment may be defined and/or measured using several parameters that reflect the typical behavior of these movements in real-world scenarios. For definition of natural movements, natural head movements may be characterized by the range, speed, and/or smoothness with which users typically move their heads when engaging with their environment. This may include nodding, turning the head left or right, tilting, and/or the combination of these movements during tasks, such as scanning a room or focusing on different objects in the VR environment.

Natural eye movements may be defined by saccades (quick jumps of the eye between fixation points), fixations (periods where the eyes are stationary and focused on a single point), and/or smooth pursuit (the eye's ability to track a moving object). The parameters may include saccadic velocity, fixation duration, and/or the accuracy of smooth pursuit. Head movements may be measured using gyroscopes and accelerometers embedded in the VR headset. The system may record the angular velocity and acceleration of the head in three axes (pitch, yaw, and roll) and/or compare these metrics against established norms for natural head movements. Eye movements may be measured using infrared eye-tracking technology that monitors the position and movement of the eyes within the VR headset. The system may capture data on saccadic movements, including their amplitude, velocity, and frequency, as well as fixation stability and duration. Smooth pursuit may be measured by tracking the eye's ability to follow a moving target with minimal lag or deviation.

Referring back to FIG. 12A, the computer device 140 continuously monitors (e.g., in step 1206) (e.g., using the tracking module 1144), using the eye-tracking sensors, user eye movements and behavior (e.g., the eye movements and/or behavior 1146) (e.g., in response to simulated conditions in the environment). Referring to FIG. 12B, in some embodiments, the eye-tracking sensors include (e.g., in step 1212) infrared cameras with a sampling rate of 200 Hz or higher and/or sub-degree precision in tracking gaze direction with latency under 10 ms. The eye-tracking sensors may include infrared cameras that capture images of the user's eyes at a high frame rate (e.g., 200 Hz or higher). These images may be processed in real-time using computer vision algorithms to extract features, such as pupil position, corneal reflection, and/or eyelid position. From these features, various eye movement metrics may be calculated, including, for example, saccades, fixations, blinks, and pupil dilation. Referring to FIG. 12C, in some embodiments, monitoring the user eye movements and behavior includes tracking (e.g., in step 1214) blink rate, blink duration, pupil dilation, and fixation stability. Referring to FIG. 12C, in some embodiments, monitoring user eye movements and behavior and adjusting visual complexity occur (e.g., in step 1216) in real-time with a latency of less than 100 milliseconds.

Calibrated eye-tracking systems may be used. For example, eye-tracking systems may be calibrated for each user to account for individual differences in eye physiology, such as interpupillary distance (IPD) and eye dominance. Calibration may help ensure that the system accurately tracks the user's gaze direction, fixation points, and saccadic movements. In environments where high accuracy is paramount, redundant tracking systems (e.g., combining inside-out tracking with external cameras) may be employed. This redundancy may help cross-verify data and correct any potential inaccuracies caused by a single tracking method. The VR system may continuously monitor the tracking data in real time to detect and/or correct any anomalies. For example, if the system detects a sudden, unrealistic jump in eye movement, the system may prompt a recalibration or discard the aberrant data to maintain the accuracy of the test results.

Referring back to FIG. 12A, the computer device 140 detects (e.g., in step 1208) eye fatigue (e.g., the eye fatigue 1152) (e.g., using the evaluation/measurement/adjustment module 1150) based on the user eye movements and behavior. This detection process may include, for example, analyzing the eye-tracking data using machine learning algorithms trained on datasets of eye movement patterns associated with different levels of fatigue. The system may use a combination of rule-based heuristics and statistical models to classify the current state of the user's eyes. Referring to FIG. 12D, in some embodiments, detecting eye fatigue includes detecting (e.g., in step 1218) signs of visual fatigue based on changes in eye-tracking metrics. Increased blink rate, longer blinks, prolonged pupil dilation and/or reduced fixation stability may indicate eye fatigue. Some embodiments map eye-tracking indicators to eye fatigue as follows: increased blink rate often indicates eye strain; longer blinks suggest fatigue, as the eyes struggle to remain open; prolonged dilation can indicate discomfort or cognitive load; and/or reduced stability is associated with fatigue and difficulty in maintaining focus. There may be differences in indicators or fatigue levels for various applications. For example, for education, fatigue is often linked to text density and screen time. For gaming, fatigue may be influenced by fast-paced motion and high contrast. For professional training, complexity and precision of visual tasks may determine fatigue levels.

Referring back to FIG. 12A, the computer device 140 dynamically adjusts (e.g., in step 1210) (e.g., using the evaluation/measurement/adjustment module 1150) the visual complexity of the VR user interface based on the detected eye fatigue. This adjustment process may include, for example, modifying various parameters of the rendered environment, such as texture resolution, polygon count, and/or lighting complexity, and the number of moving objects. The system may use, for example, a predefined set of complexity levels or a continuous scale, adjusting the visual elements gradually to avoid jarring transitions. These adjustments may be made in real-time, with the rendering engine applying the changes within a single frame or over a short sequence of frames to ensure smooth visual transitions, for example. Referring to FIG. 12E, in some embodiments, the computer device 140 adjusts (e.g., in step 1220) visual complexity gradually so as to avoid abrupt changes that may disrupt user experience. In some embodiments, in interactive VR scenarios, for example, the computer device 140 adjusts (e.g., in step 1222) visual complexity by modifying text size, reading speed, and visual complexity of diagrams in educational modules, and/or modulating difficulty levels, number and concentration of non-player characters (NPC density), and/or environmental effects in gaming environments.

Referring to FIG. 12F, in some embodiments, the computer device 140 dynamically adjusts (e.g., in step 1218) the visual complexity by reducing texture resolution, decreasing contrast, simplifying visual details, and/or dimming bright areas. In some embodiments, the computer device 140 dynamically adjusts (e.g., in step 1226) the visual complexity based on a context selected from the group consisting of: education, gaming, and professional training (e.g., with context-specific adjustments based on fatigue indicators). In some embodiments, in a virtual classroom setting, for example, adjustments may include (e.g., in step 1228) reducing text density, increasing line spacing, and/or simplifying background visuals. In some embodiments, in a gaming environment, for example, adjustments may include (e.g., in step 1230) lowering texture resolution, reducing brightness and dynamic lighting effects, and/or smoothing or slowing down motion effects.

The texture resolution reduction may be implemented using mipmap levels, dynamically selecting lower resolution textures based on the fatigue level, for example. Contrast reduction can be achieved by adjusting the tone mapping parameters in the rendering pipeline. Visual detail simplification may include, for example, switching to lower polygon models or disabling certain post-processing effects. Dimming bright areas may be implemented by adjusting the exposure settings in the virtual camera or modifying the emission properties of light sources in the scene.

Referring to FIG. 12G, in some embodiments, the computer device 140 also adjusts (e.g., in step 1232) task complexity by reducing the number of simultaneous visual elements based on the detected eye fatigue. This step may include, for example, dynamically culling non-essential objects from the scene, reducing the number of moving elements, and/or simplifying user interface components. The system may maintain a priority queue of visual elements, removing lower priority items as fatigue increases, for example. In some embodiments, the computer device 140 also uses (e.g., in step 1234) one or more algorithms for pattern recognition to detect signs of fatigue and/or visual scene simplification to gradually reduce visual complexity and/or insert micro-breaks (e.g., brief pauses in gameplay). In some embodiments, the computer device 140 also generates (e.g., in step 1236) a comprehensive report on visual endurance, which may include, for example, insights on fatigue progression, optimal screen time recommendations, and/or personalized adjustments. In some embodiments, the computer device 140 also calibrates and/or validates (e.g., in step 1238) the system using a control group to establish baseline measurements of eye movements and visual performance. Some embodiments gather data from a diverse group of users, compare it against fatigue indicators, and/or refine algorithms. In some embodiments, the computer device 140 also generates (e.g., in step 1240) recommendations for optimal VR usage durations, which may include, for example, session limits, specific break intervals, and/or visual settings tailored to the user's endurance profile.

Referring to FIG. 12H, in some embodiments, the computer device 140 also establishes (e.g., in step 1242) baseline eye fatigue levels for the user, compares (e.g., in step 1244) real-time eye tracking data to the baseline levels, and/or initiates (e.g., in step 1246) visual complexity adjustments when deviations from the baseline exceed predetermined thresholds. The baseline establishment process may include a calibration session where the user's eye movements are recorded during a series of standardized visual tasks. This data may then be used to create a personalized fatigue model for the user. During active use, the system may, for example, continuously compare current eye tracking metrics to this baseline, using statistical methods to detect significant deviations.

In some embodiments, the computer device 140 also allows (e.g., in step 1248) user input to fine-tune the sensitivity of fatigue detection and the degree of visual adjustments, stores (e.g., in step 1250) user preferences for future VR sessions, and/or adapts (e.g., in step 1252) the system's response to eye fatigue based on accumulated user data over multiple sessions. The user preferences and historical data may be stored in a secure database, with the system using machine learning techniques, for example, to refine its fatigue detection and adjustment strategies over time. This adaptive method can help ensure that the system becomes more personalized and effective with continued use.

### VR Real-Time Visual Health Monitoring System

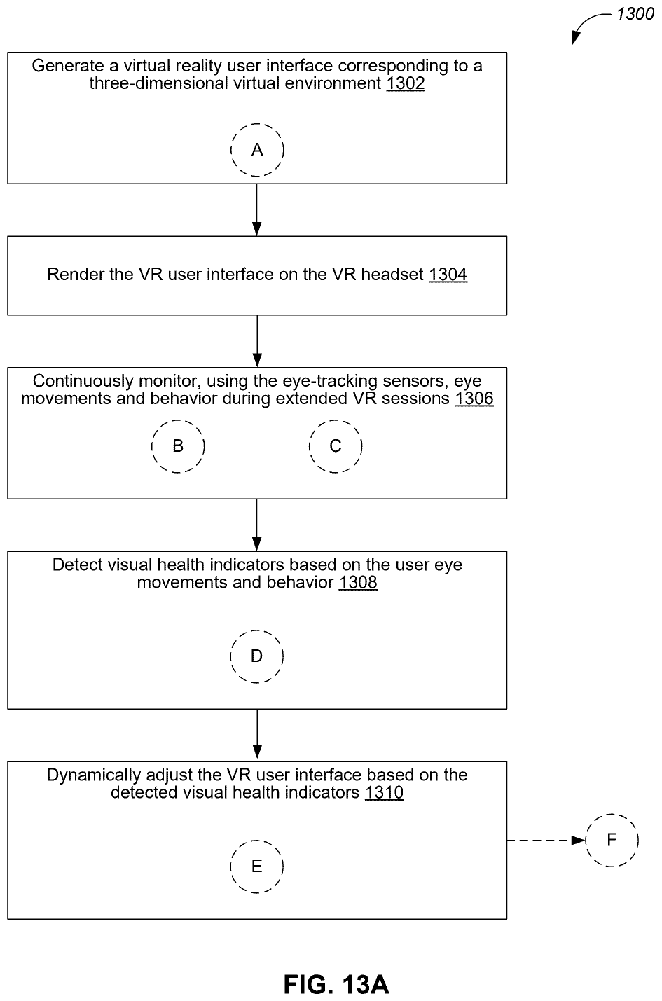

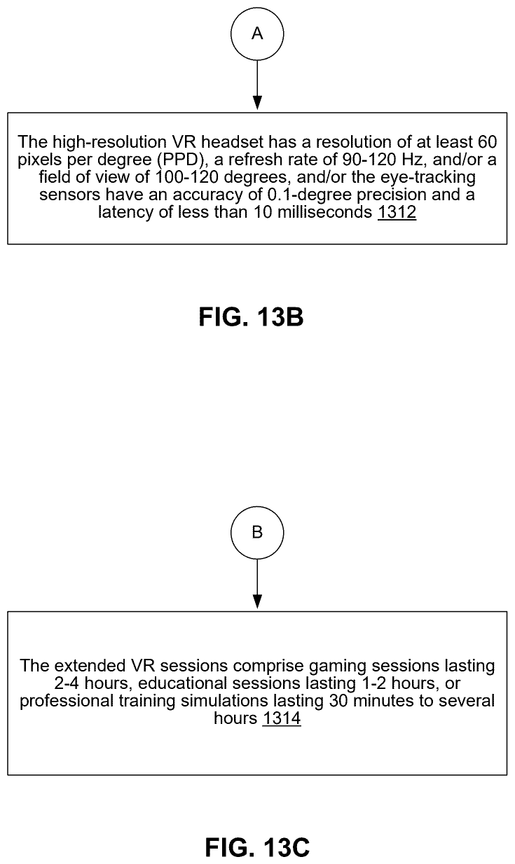

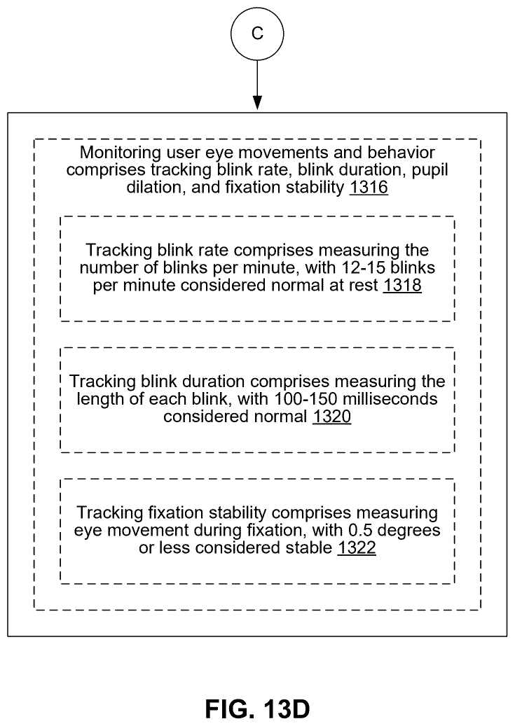

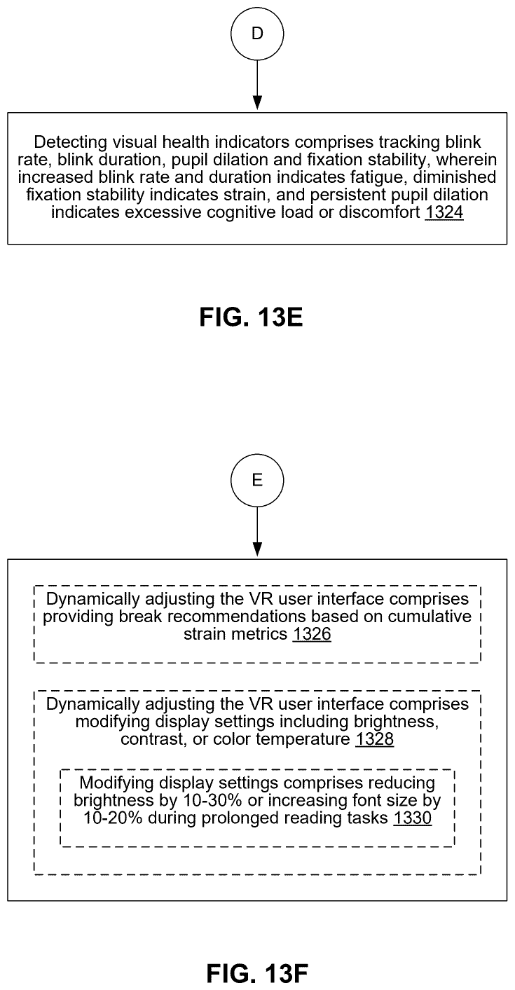

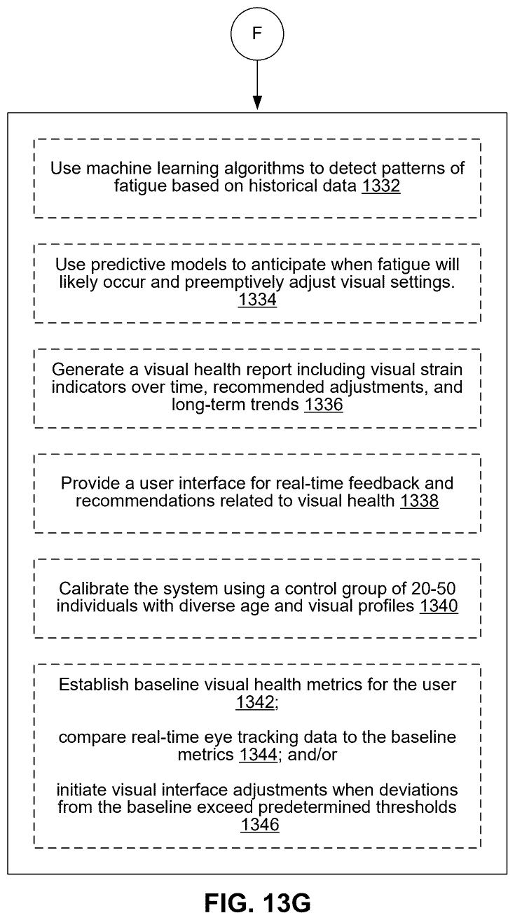

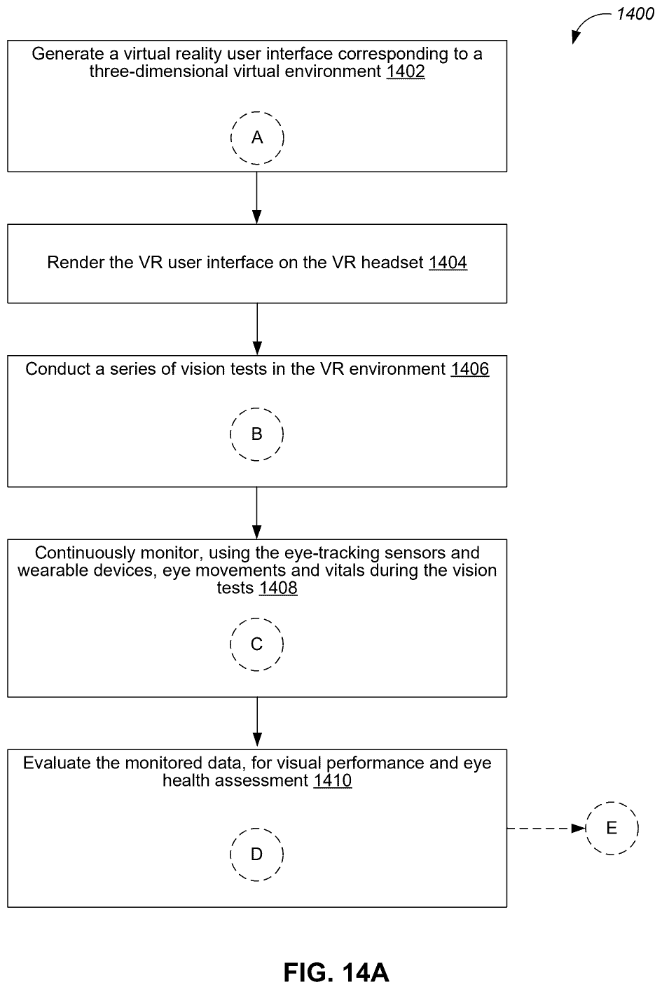

According to some embodiments, the vision test system 1100 described above is configured to implement a method for real-time visual health monitoring during extended use. FIGS. 13A-13G show a flow diagram of an example process 1300 for real-time visual health monitoring during extended use, according to some embodiments. Traditional methods involve intermittent breaks, static exercises, and questionnaires, lacking real-time feedback. This is inadequate for detecting early signs of visual strain or adjusting content dynamically to mitigate potential issues. Accordingly, the techniques described herein address at least some of these problems.

The computer device 140 (e.g., the computing device described above in reference to FIGS. 11A and 11B) generates (e.g., in step 1302) (e.g., using the UI module 1134) a VR user interface corresponding to a three-dimensional virtual environment.

The computer device 140 also renders (e.g., in step 1304) (e.g., using the rendering module 1138) the VR user interface on the VR headset (e.g., on the HMD 312A). Example details of the three-dimensional virtual environment and rendering the VR user interface are described above in reference to FIG. 12B, according to some embodiments.

The computer device 140 also continuously monitors (e.g., in step 1306) (e.g., using the tracking module 1144), using the eye-tracking sensors, eye movements and/or behavior (e.g., the eye movements and/or vitals 1146, and the responses and/or behavior 1148) during extended VR sessions. In some embodiments, (e.g., in step 1312, FIG. 13B) the high-resolution VR headset has a resolution of at least 60 pixels per degree (PPD), a refresh rate of 90-120 Hz, and/or a field of view of 100-120 degrees, and/or the eye-tracking sensors have an accuracy of 0.1-degree precision and/or a latency of less than 10 milliseconds. In some embodiments, the extended VR sessions include (e.g., in step 1314, FIG. 13C) gaming sessions lasting 2-4 hours, educational sessions lasting 1-2 hours, and/or professional training simulations lasting 30 minutes to several hours.

Referring to FIG. 13D, in some embodiments, the computer device 140 monitors (e.g., in step 1316) user eye movements and/or behavior by tracking blink rate, blink duration, pupil dilation, and/or fixation stability. In some embodiments, the computer device 140 tracks (e.g., in step 1318) blink rate by measuring the number of blinks per minute, with 12-15 blinks per minute considered normal at rest. In some embodiments, the computer device 140 tracks (e.g., in step 1320) blink duration by measuring the length of each blink, with 100-150 milliseconds considered normal. In some embodiments, the computer device 140 tracks pupil dilation by measuring pupil size, with 2-4 millimeters considered normal. In some embodiments, the computer device 140 tracks (e.g., in step 1322) fixation stability by measuring eye movement during fixation, with 0.5 degrees or less considered stable.

Referring back to FIG. 13A, the computer device 140 also detects (e.g., in step 1306) (e.g., using the evaluation/measurement/adjustment module 1150) visual health indicators (e.g., the visual health 1156) based on the user eye movements and behavior. Referring to FIG. 13E, in some embodiments, the computer device 140 detects (e.g., in step 1324) visual health indicators by tracking blink rate, blink duration, pupil dilation and/or fixation stability. The computer device 140 interprets, for example, increased blink rate and/or duration as indicating fatigue, diminished fixation stability as indicating strain, and/or persistent pupil dilation as indicating excessive cognitive load and/or discomfort.

Referring back to FIG. 13A, the computer device 140 also dynamically adjusts (e.g., in step 1308) the VR user interface based on the detected visual health indicators. Referring to FIG. 13F, in some embodiments, the computer device 140 dynamically adjusts (e.g., in step 1326) the VR user interface by providing break recommendations based on cumulative strain metrics. In some embodiments, the computer device 140 dynamically adjusts (e.g., in step 1328) the VR user interface by modifying display settings including brightness, contrast, and/or color temperature. In some embodiments, the computer device 140 modifies (e.g., in step 1330) display settings by reducing brightness by 10-30% and/or increasing font size by 10-20% during prolonged reading tasks.

Referring to FIG. 13G, in some embodiments, the computer device 140 uses (e.g., in step 1332) machine learning algorithms to detect patterns of fatigue based on historical data. In some embodiments, the computer device 140 uses (e.g., in step 1334) predictive models to anticipate when fatigue will likely occur and/or preemptively adjusts visual settings. In some embodiments, the computer device 140 generates (e.g., in step 1336) a visual health report including visual strain indicators over time, recommended adjustments, and/or long-term trends. In some embodiments, the computer device 140 provides (e.g., in step 1338) a user interface for real-time feedback and/or recommendations related to visual health. In some embodiments, the computer device 140 calibrates (e.g., in step 1340) the system using a control group of 20-50 individuals with diverse age and/or visual profiles, establishes (e.g., in step 1342) baseline visual health metrics for the user, compares (e.g., in step 1344) real-time eye tracking data to the baseline metrics, and/or initiates (e.g., in step 1346) visual interface adjustments when deviations from the baseline exceed predetermined thresholds.

Example details of various steps described herein are further described above in reference to FIGS. 12A-12H, according to some embodiments.

### VR-Based Vision Testing and Eye Health Monitoring System

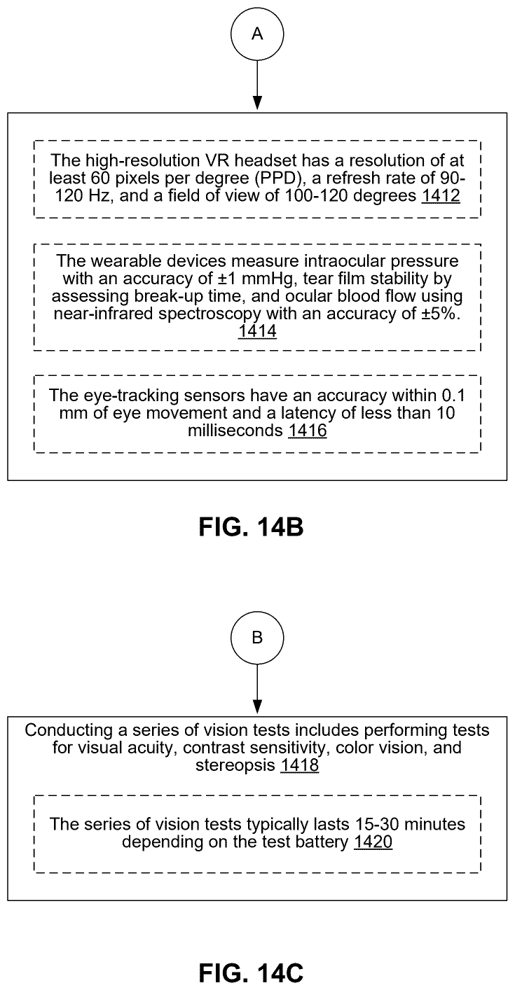

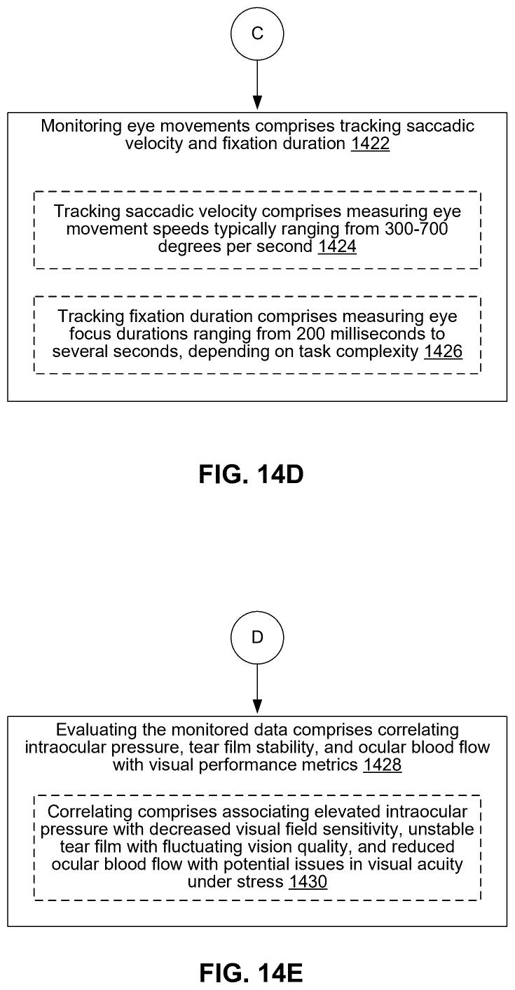

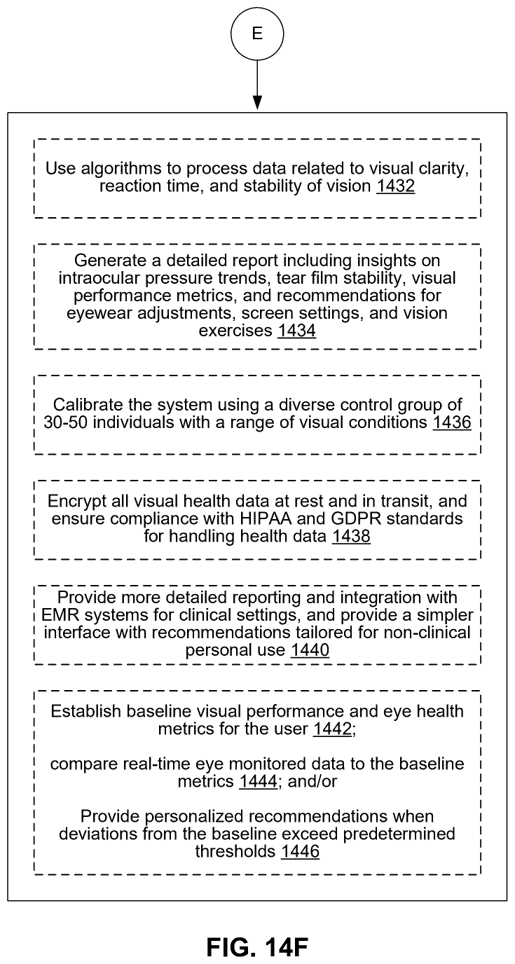

According to some embodiments, the vision test system 1100 described above is configured for vision testing and eye health monitoring. FIGS. 14A-14F show a flow diagram of an example process 1400 for vision testing and eye health monitoring, according to some embodiments. Traditional vision tests include Snellen charts, intraocular pressure tests (e.g., using tonometry), and tear film assessments. These tests are usually performed in clinical settings and do not offer the immersive or dynamic environment provided by VR-based systems. Accordingly, the techniques described herein address at least some of these problems.

The computer device 140 (e.g., the computing device described above in reference to FIGS. 11A and 11B) generates (e.g., in step 1402) (e.g., using the UI module 1134) a VR user interface corresponding to a three-dimensional virtual environment (e.g., the environment 1136). In some embodiments, the computer device 140 also includes wearable device(s) for measuring intraocular pressure, tear film stability, and/or ocular blood flow. Referring to FIG. 14B, in some embodiments, the high-resolution VR headset has (e.g., in step 1412) a resolution of at least 60 pixels per degree (PPD), a refresh rate of 90-120 Hz, and/or a field of view of 100-120 degrees. In some embodiments, the wearable devices measure (e.g., in step 1414) intraocular pressure with an accuracy of ±1 mmHg, tear film stability by assessing break-up time, and/or ocular blood flow using near-infrared spectroscopy with an accuracy of ±5%. In some embodiments, the eye-tracking sensors have (e.g., in step 1416) an accuracy within 0.1 mm of eye movement and/or a latency of less than 10 milliseconds.

The computer device 140 also renders (e.g., in step 1404) (e.g., using the rendering module 1138) the VR user interface on the VR headset (e.g., HMD 312A). Example details of the three-dimensional virtual environment and rendering the VR user interface are described above in reference to FIG. 12B, according to some embodiments.

The computer device 140 also conducts (e.g., in step 1406) (e.g., using the simulation module 1140) a series of vision tests (e.g., the test sequences 1142) in the VR user interface (e.g., within a VR environment presented in the VR user interface). Referring to FIG. 14C, in some embodiments, the computer device 140 conducts (e.g., in step 1418) a series of vision tests by performing tests for visual acuity, contrast sensitivity, color vision, and/or stereopsis. In some embodiments, the series of vision tests typically lasts (e.g., in step 1420) 15-30 minutes depending on test battery.

The computer device 140 also continuously monitors (e.g., in step 1408) (e.g., using the tracking module 1144), using the eye-tracking sensors and the wearable devices, eye movements and vitals (e.g., the eye movements and/or vitals 1146) during the vision tests. Referring to FIG. 14D, in some embodiments, the computer device 140 monitors (e.g., in step 1422) eye movements by tracking saccadic velocity and/or fixation duration. In some embodiments, the computer device 140 tracks (e.g., in step 1424) saccadic velocity by measuring eye movement speeds typically ranging from 300-700 degrees per second. In some embodiments, the computer device 140 tracks (e.g., in step 1426) fixation duration by measuring eye focus durations ranging from 200 milliseconds to several seconds, depending on task complexity.

Referring again to FIG. 14A, the computer device 140 also evaluates (e.g., in step 1410) the monitored data for vision performance (e.g., the vision performance 1154) and eye health assessment (e.g., the visual health 1156). Referring to FIG. 14E, in some embodiments, the computer device 140 evaluates (e.g., in step 1428) the monitored data by correlating intraocular pressure, tear film stability, and/or ocular blood flow with visual performance metrics. In some embodiments, the computer device 140 correlates (e.g., in step 1430) by associating elevated intraocular pressure with decreased visual field sensitivity, unstable tear film with fluctuating vision quality, and/or reduced ocular blood flow with potential issues in visual acuity under stress.

Referring to FIG. 14F, in some embodiments, the computer device 140 uses (e.g., in step 1432) algorithms to process data related to visual clarity, reaction time, and/or stability of vision. In some embodiments, the computer device 140 generates (e.g., in step 1434) a detailed report including insights on intraocular pressure trends, tear film stability, visual performance metrics, and/or recommendations for eyewear adjustments, screen settings, and/or vision exercises. In some embodiments, the computer device 140 compares (e.g., in step 1436) monitored data against established clinical thresholds to flag potential issues, such as intraocular pressure exceeding 21 mmHg for glaucoma risk. In some embodiments, the computer device 140 calibrates (e.g., in step 1436) the system using a diverse control group of 30-50 individuals with a range of visual conditions. In some embodiments, the computer device 140 (e.g., in step 1438) encrypts all visual health data at rest and/or in transit, and/or ensures compliance with HIPAA and/or GDPR standards for handling health data. In some embodiments, the computer device 140 provides (e.g., in step 1440) more detailed reporting and/or integration with EMR systems for clinical settings, and/or provides (e.g., in step 1440) a simpler interface with recommendations tailored for non-clinical personal use. In some embodiments, the computer device 140 also establishes (e.g., in step 1442) baseline visual performance and/or eye health metrics for the user, compares (e.g., in step 1444) real-time monitored data to the baseline metrics, and/or provides (e.g., in step 1446) personalized recommendations when deviations from the baseline exceed predetermined thresholds.

Example details of various steps described herein are further described above in reference to FIGS. 12A-12H, according to some embodiments.

### VR Eye Strain Identification System

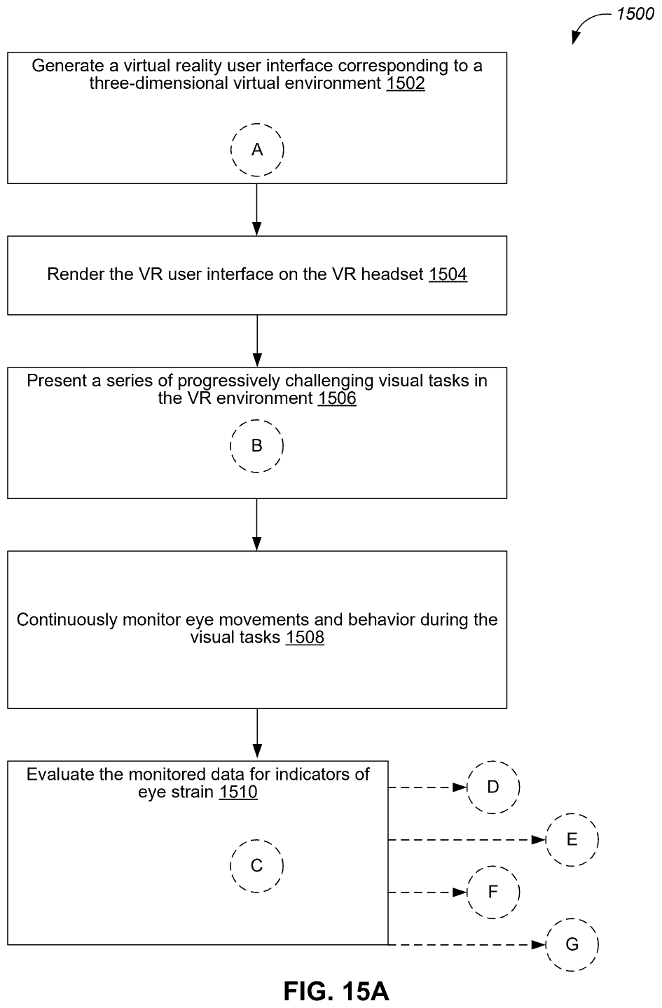

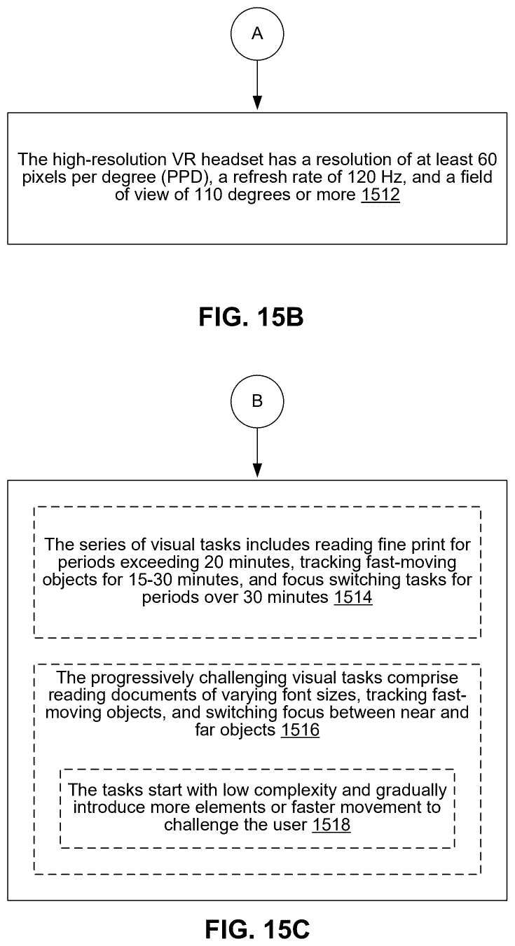

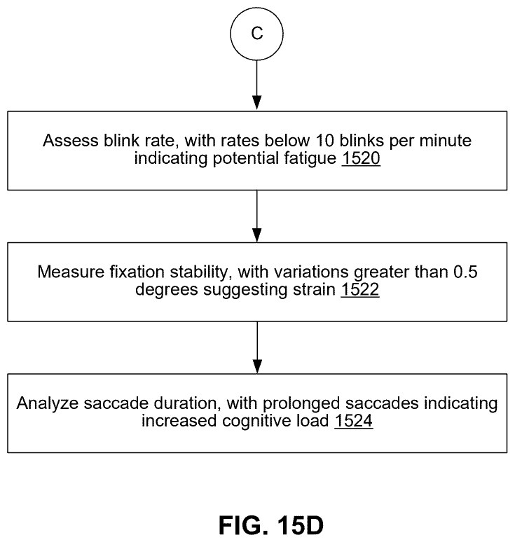

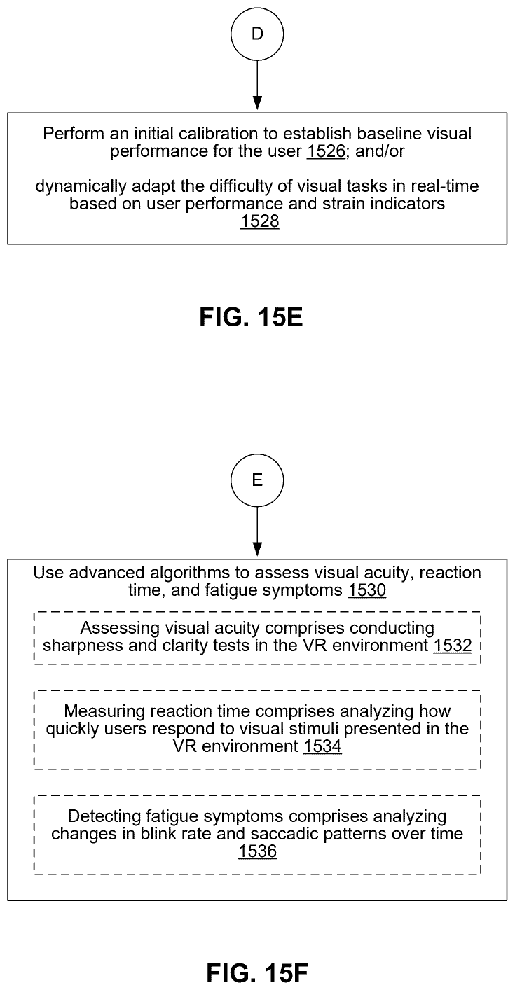

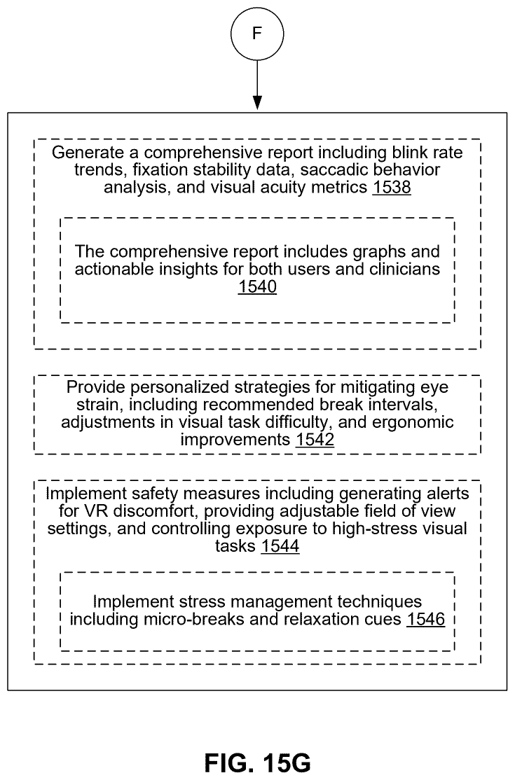

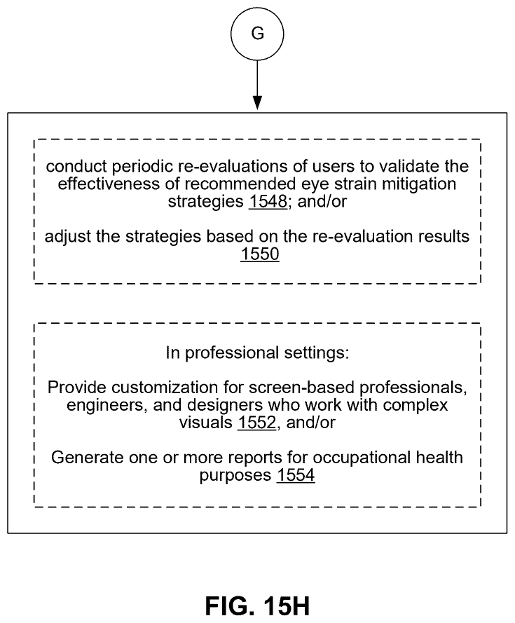

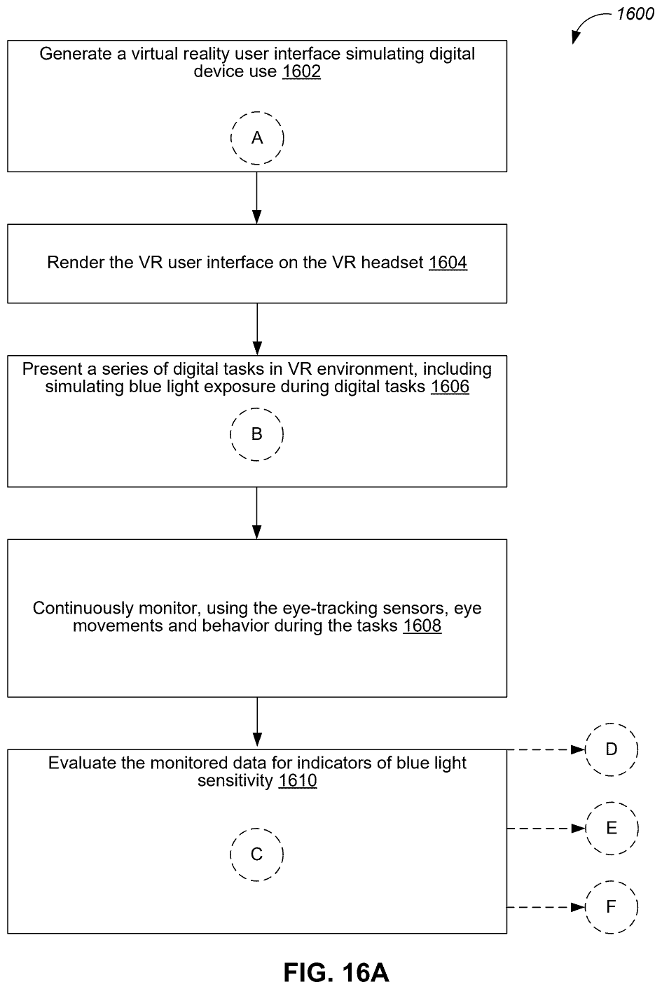

According to some embodiments, the vision test system 1100 described above is configured for identifying potential eye strain issues through prolonged engagement. FIGS. 15A-15H show a flow diagram of an example process 1500 for identifying potential eye strain issues through prolonged engagement, according to some embodiments. Traditional methods include manual observation, subjective feedback, and static eye tests (e.g., Schirmer's test, visual acuity tests). These methods are not well-suited for dynamic environments or prolonged digital use. Accordingly, the techniques described herein address at least some of these problems.

The computer device 140 (e.g., the computing device described above in reference to FIGS. 11A and 11B) generates (e.g., in step 1502) (e.g., using the UI module 1134) a VR user interface corresponding to a three-dimensional virtual environment (e.g., the environment 1136). Referring next to FIG. 15B, in some embodiments, (e.g., in step 1512) the high-resolution VR headset has a resolution of at least 60 pixels per degree (PPD), a refresh rate of 120 Hz, and/or a field of view of 110 degrees or more.

Referring back to FIG. 15A, the computer device 140 also renders (e.g., in step 1504) (e.g., using the rendering module 1138) the VR user interface on the HMD 312A. Example details of the three-dimensional virtual environment and rendering the VR user interface are described above in reference to FIG. 12B, according to some embodiments.

The computer device 140 also presents (e.g., in step 1506) (e.g., using the simulation module 1140) a series of progressively challenging visual tasks (e.g., the scenarios 1142) in the VR user interface. Referring next to FIG. 15C, in some embodiments, the series of visual tasks includes (e.g., in step 1514) reading fine print for periods exceeding 20 minutes, tracking fast-moving objects for 15-30 minutes, and/or focus switching tasks for periods over 30 minutes. In some embodiments, the progressively challenging visual tasks include (e.g., in step 1516) reading documents of varying font sizes, tracking fast-moving objects, and/or switching focus between near and/or far objects. In some embodiments, the computer device 140 (e.g., in step 1518) starts the tasks with low complexity and/or gradually introduces more elements and/or faster movement to challenge the user.

Referring back to FIG. 15A, the computer device 140 also continuously monitors (e.g., in step 1508) (e.g., using the tracking module 1144), using the eye-tracking sensors, eye movements and/or behavior (e.g., the eye movements 1146, the responses 1148) during the visual tasks.

Referring back to FIG. 15A, the computer device 140 also evaluates (e.g., in step 1510) (e.g., using the evaluation/measurement module 1150) the monitored data for indicators of eye strain (e.g., the eye strain 1158). Referring next to FIG. 15D, in some embodiments, the computer device 140 evaluates the monitored data by assessing (e.g., in step 1520) blink rate, with rates below 10 blinks per minute indicating potential fatigue, measuring (e.g., in step 1522) fixation stability, with variations greater than 0.5 degrees suggesting strain, and/or analyzing (e.g., in step 1524) saccade duration, with prolonged saccades indicating increased cognitive load.

Referring next to FIG. 15E, in some embodiments, the computer device 140 also performs (e.g., in step 1526) an initial calibration to establish baseline visual performance for the user, and/or dynamically adapts (e.g., in step 1528) the difficulty of visual tasks in real-time based on user performance and/or strain indicators.

Referring next to FIG. 15F, in some embodiments, the computer device 140 also uses (e.g., in step 1530) one or more algorithms to assess visual acuity, reaction time, and/or fatigue symptoms. In some embodiments, the computer device 140 assesses (e.g., in step 1532) visual acuity by conducting sharpness and/or clarity tests in the VR environment. In some embodiments, the computer device 140 measures (e.g., in step 1534) reaction time by analyzing how quickly users respond to visual stimuli presented in the VR environment. In some embodiments, the computer device 140 detects (e.g., in step 1536) fatigue symptoms by analyzing changes in blink rate and/or saccadic patterns over time.

Referring next to FIG. 15G, in some embodiments, the computer device 140 also generates (e.g., in step 1538) a comprehensive report including blink rate trends, fixation stability data, saccadic behavior analysis, and/or visual acuity metrics. In some embodiments, the comprehensive report includes (e.g., in step 1540) graphs and/or actionable insights for both users and/or clinicians. In some embodiments, the computer device 140 provides (e.g., in step 1542) personalized strategies for mitigating eye strain (e.g., based on individual's visual capacity and/or history of eye strain), including recommended break intervals, adjustments in visual task difficulty, and/or ergonomic improvements. In some embodiments, the computer device 140 implements (e.g., in step 1544) safety measures including generating alerts for VR discomfort and/or motion sickness, providing adjustable field of view settings, and/or controlling exposure to high-stress visual tasks. In some embodiments, the computer device 140 implements (e.g., in step 1546) stress management techniques including micro-breaks and/or relaxation cues.

Referring next to FIG. 15H, in some embodiments, the computer device 140 also conducts (e.g., in step 1548) periodic re-evaluations of users to validate the effectiveness of recommended eye strain mitigation strategies; and/or adjusts (e.g., in step 1550) the strategies based on the re-evaluation results. In some embodiments, in professional settings, the computer device 140 provides (e.g., in step 1552) customization for screen-based professionals, engineers, and/or designers who work with complex visuals, and/or generates (e.g., in step 1554) one or more reports for occupational health purposes.

Example details of various steps described herein are further described above in reference to FIGS. 12A-12H, according to some embodiments.

### VR Blue Light Sensitivity Evaluation System

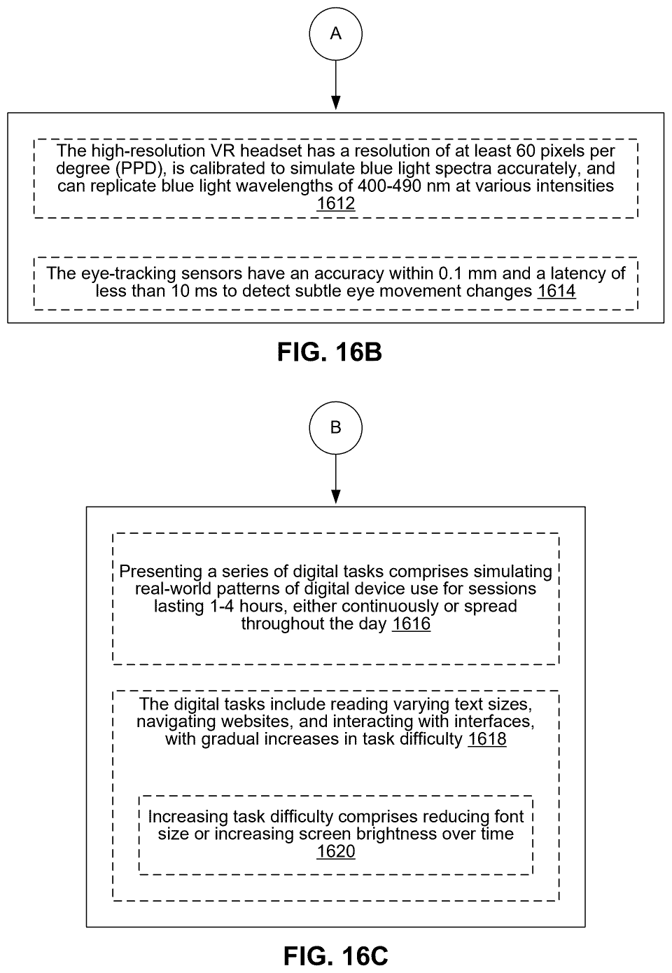

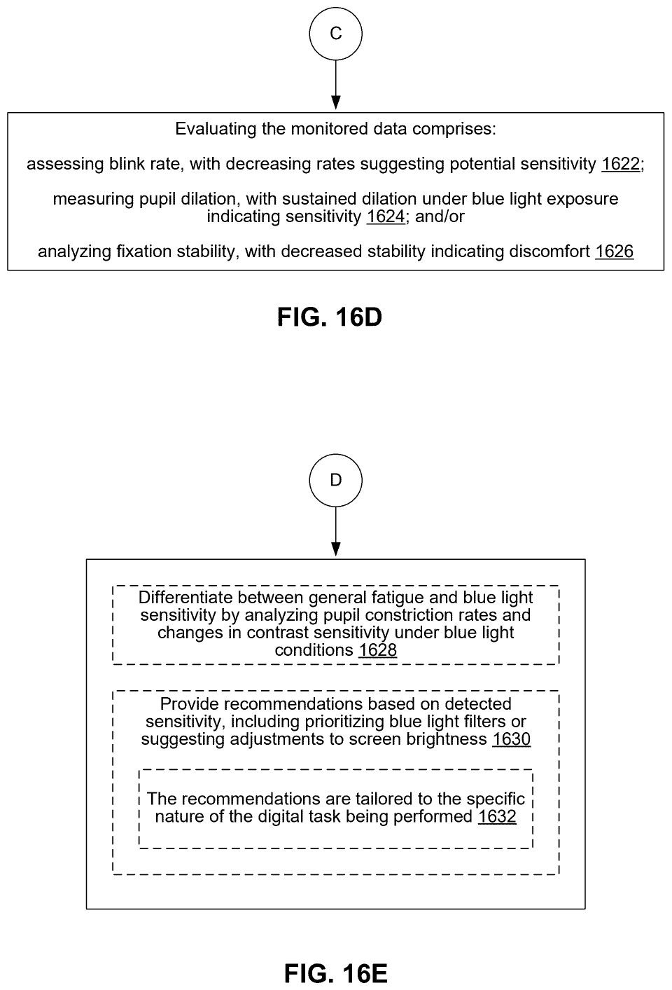

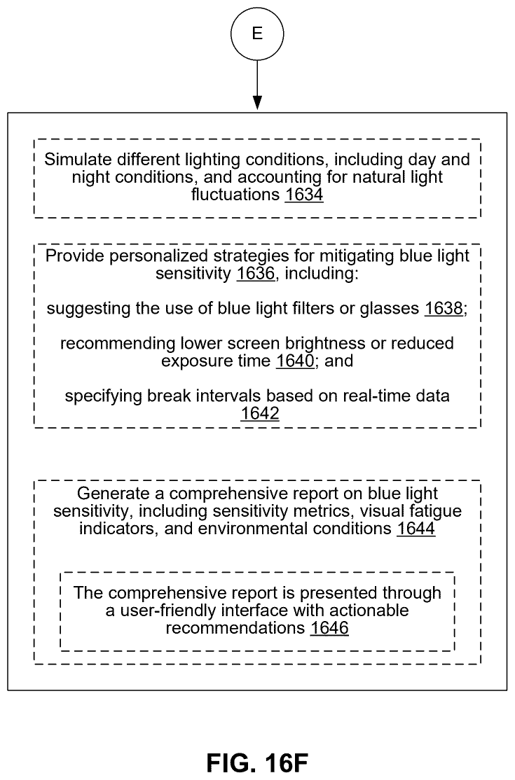

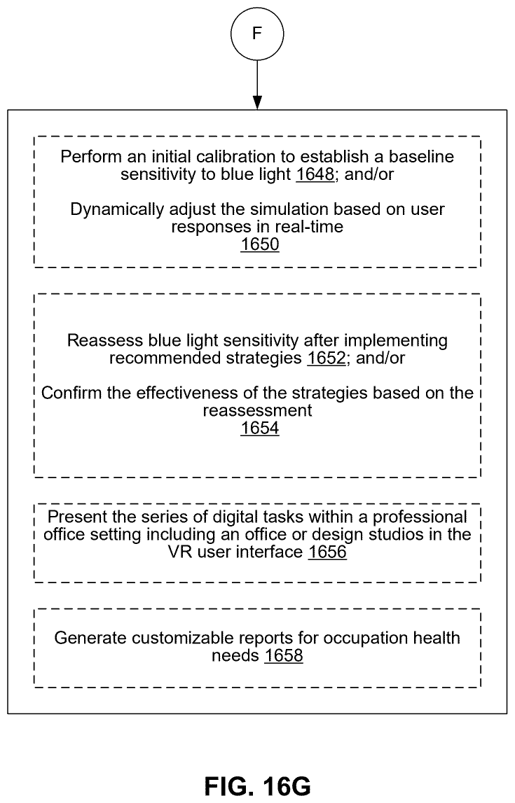

According to some embodiments, the vision test system 1100 described above is configured for evaluating vision during digital device use and identifying blue light sensitivity. FIGS. 16A-16G show a flow diagram of an example process 1600 for evaluating vision during digital device use and identifying blue light sensitivity, according to some embodiments. Traditional methods involve static blue light exposure tests and questionnaires about screen time habits. These are often not personalized and lack real-time monitoring of visual fatigue. Accordingly, the techniques described herein address at least some of the problems described herein.

The computer device 140 (e.g., the computing device described above in reference to FIGS. 11A and 11B) generates (e.g., in step 1602) (e.g., using the UI module 1134) a VR user interface simulating digital device use (e.g., the environment 1136). The digital device use can be the use of a personal computer, a laptop, and/or a mobile device, for example. Referring next to FIG. 16B, in some embodiments, (e.g., in step 1612) the high-resolution VR headset has a resolution of at least 60 pixels per degree (PPD), is calibrated to simulate blue light spectra accurately, and/or can replicate blue light wavelengths of 400-490 nm at various intensities. In some embodiments, (e.g., in step 1614) the eye-tracking sensors have an accuracy within 0.1 mm and/or a latency of less than 10 ms to detect subtle eye movement changes.

The computer device 140 also renders (e.g., in step 1604) (e.g., using the rendering module 1138) the VR user interface on the HMD 312A. Example details of the three-dimensional virtual environment and rendering the VR user interface are described above in reference to FIG. 12B, according to some embodiments.

The computer device 140 also presents (e.g., in step 1606) (e.g., using the simulation module 1140), in the VR user interface, a series of digital tasks (e.g., the scenarios 1142), including simulating blue light exposure during the digital tasks. The digital tasks may include, for example, browsing websites, reading an article on the web, coding, and/or similar online activities. Referring next to FIG. 16C, in some embodiments, the computer device 140 presents (e.g., in step 1616) a series of digital tasks by simulating real-world patterns of digital device use for sessions lasting 1-4 hours, either continuously and/or spread throughout the day. In some embodiments, the digital tasks include (e.g., in step 1618) reading varying text sizes, navigating websites, and/or interacting with interfaces, with gradual increases in task difficulty. In some embodiments, the computer device 140 increases (e.g., in step 1620) task difficulty by reducing font size and/or increasing screen brightness over time.

Referring back to FIG. 16A, the computer device 140 also continuously monitors (e.g., in step 1608) (e.g., using the tracking module 1144), using the eye-tracking sensors, eye movements and/or behavior (e.g., the eye movements 1146, the responses 1148) during the digital tasks.

Referring back to FIG. 16A, the computer device 140 also evaluates (e.g., in step 1610) (e.g., using the evaluation/measurement module 1150) the monitored data for indicators of blue light sensitivity (e.g., the blue light sensitivity 1160). Referring next to FIG. 16D, in some embodiments, the computer device 140 evaluates the monitored data by assesses (e.g., in step 1622) blink rate, with decreasing rates suggesting potential sensitivity, measuring (e.g., in step 1624) pupil dilation, with sustained dilation under blue light exposure indicating sensitivity, and/or analyzing (e.g., in step 1626) fixation stability, with decreased stability indicating discomfort.

Referring next to FIG. 16E, in some embodiments, the computer device 140 also differentiates (e.g., in step 1628) between general fatigue and/or blue light sensitivity by analyzing pupil constriction rates and/or changes in contrast sensitivity under blue light conditions. In some embodiments, the computer device 140 also provides (e.g., in step 1630) recommendations based on detected sensitivity, including prioritizing blue light filters and/or suggesting adjustments to screen brightness. In some embodiments, the computer device 140 tailors (e.g., in step 1632) the recommendations to the specific nature of the digital task being performed.

Referring next to FIG. 16F, in some embodiments, the computer device 140 also simulates (e.g., in step 1634) different lighting conditions, including day and/or night conditions, and/or accounts for natural light fluctuations. In some embodiments, the computer device 140 also provides (e.g., in step 1636) personalized strategies for mitigating blue light sensitivity, including, by, for example, suggesting (e.g., in step 1638) the use of blue light filters and/or glasses, recommending (e.g., in step 1640) lower screen brightness and/or reduced exposure time, and/or specifying (e.g., in step 1642) break intervals based on real-time data. In some embodiments, the computer device 140 also generates (e.g., in step 1644) a comprehensive report on blue light sensitivity, including sensitivity metrics, visual fatigue indicators, and/or environmental conditions. In some embodiments, the computer device 140 also presents (e.g., in step 1646) the comprehensive report through a user-friendly interface with actionable recommendations.

Referring next to FIG. 1G, in some embodiments, the computer device 140 also performs (e.g., in step 1648) an initial calibration to establish a baseline sensitivity to blue light, and/or dynamically adjusts (e.g., in step 1650) the simulation based on user responses in real-time. In some embodiments, the computer device 140 also reassesses (e.g., in step 1652) blue light sensitivity after implementing recommended strategies, and/or confirms (e.g., in step 1654) the effectiveness of the strategies based on the reassessment. In some embodiments, the computer device 140 also presents (e.g., in step 1656) the series of digital tasks within a professional office setting including an office and/or design studios in the VR user interface. In some embodiments, the computer device 140 also generates (e.g., in step 1658) customizable reports for occupational health needs.

Example details of various steps described herein are further described above in reference to FIGS. 12A-12H, according to some embodiments.

### VR Cognitive Load and Mental Fatigue Testing System

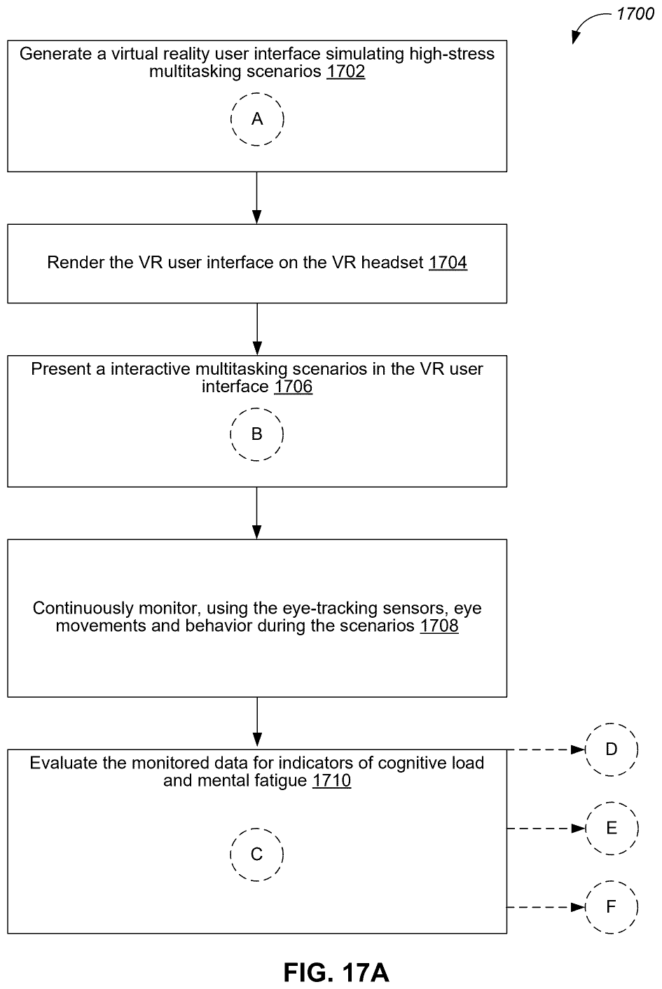

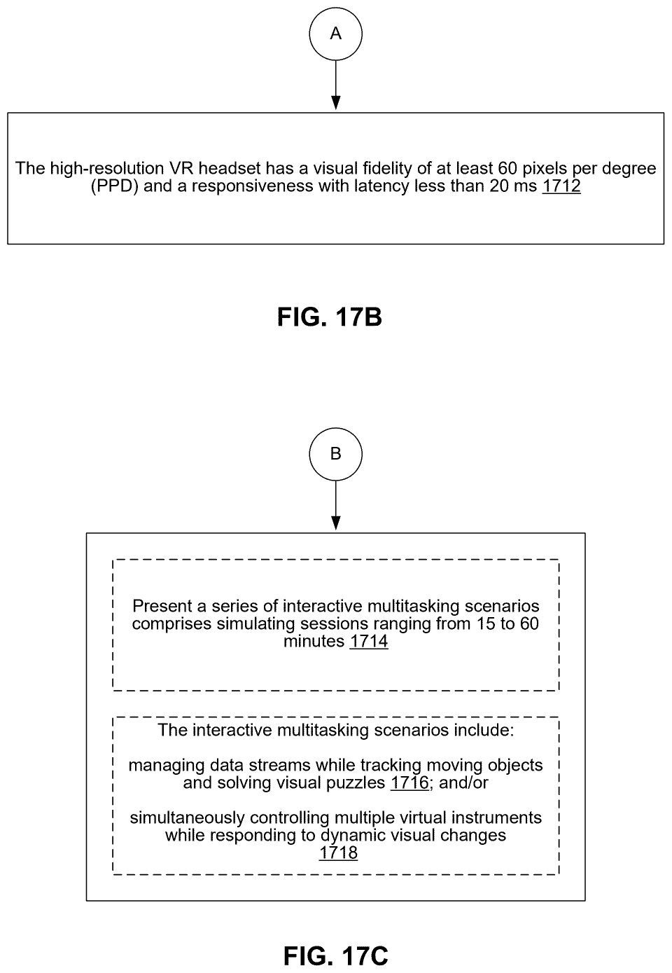

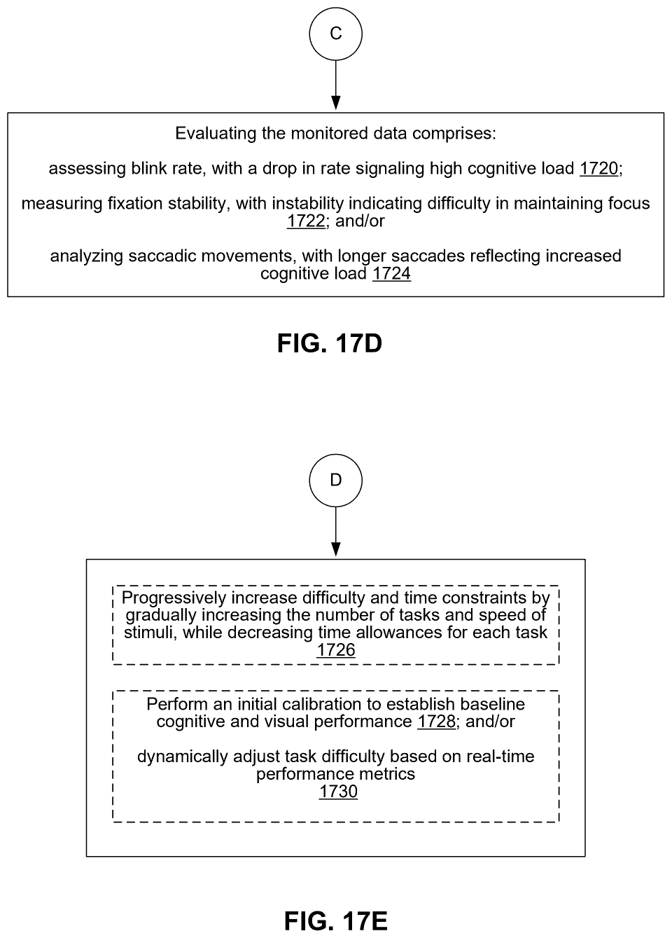

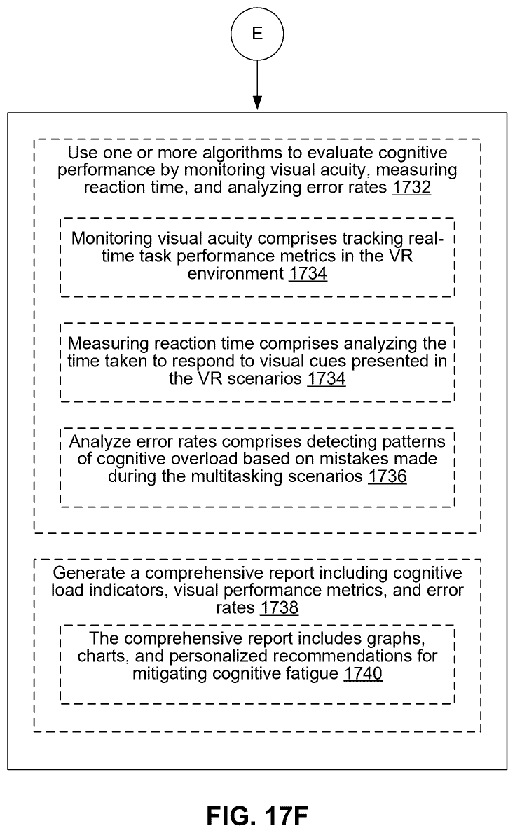

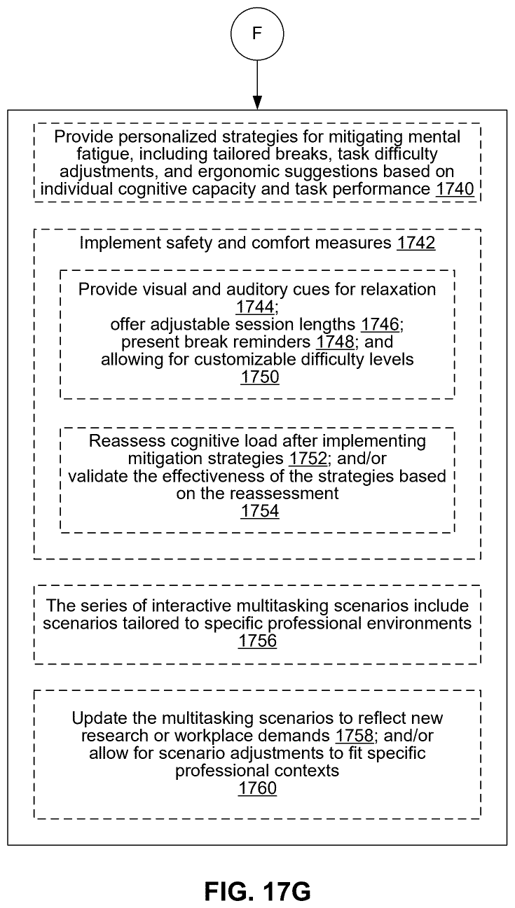

According to some embodiments, the vision test system 1100 described above is configured for testing cognitive load and mental fatigue effects on vision. FIGS. 17A-17G show a flow diagram of an example process 1700 for testing cognitive load and mental fatigue effects on vision, according to some embodiments. Traditional methods include cognitive testing (e.g., Stroop test), reaction time measurements, and self-reporting scales. These methods lack the immersive and controlled environment offered by VR. Accordingly, the techniques described herein address at least some of these problems.

The computer device 140 (e.g., the computing device described above in reference to FIGS. 11A and 11B) generates (e.g., in step 1702) (e.g., using the UI module 1134) a VR user interface simulating high-stress multitasking scenarios (e.g., the environment 1136). Referring next to FIG. 17B, in some embodiments, (e.g., in step 1712) the high-resolution VR headset has a visual fidelity of at least 60 pixels per degree (PPD) and a responsiveness with latency less than 20 ms 1712.

Referring back to FIG. 17A, the computer device 140 also renders (e.g., in step 1704) (e.g., using the rendering module 1138) the VR user interface on the VR headset (e.g., on the HMD 312A). Example details of the three-dimensional virtual environment and rendering the VR user interface are described above in reference to FIG. 12B, according to some embodiments.

The computer device 140 also presents (e.g., in step 1706) (e.g., using the simulation module 1140), in the VR user interface, a series of interactive multitasking scenarios (e.g., the scenarios 1142). Referring next to FIG. 17C, in some embodiments, the computer device 140 presents (e.g., in step 1714) a series of interactive multitasking scenarios by simulating sessions ranging from 15 to 60 minutes. In some embodiments, the interactive multitasking scenarios include managing (e.g., in step 1716) data streams while tracking moving objects and/or solving visual puzzles, and/or simultaneously controlling (e.g., in step 1718) multiple virtual instruments while responding to dynamic visual changes.

Referring back to FIG. 17A, the computer device 140 also continuously monitors (e.g., in step 1708) (e.g., using the tracking module 1144), using the eye-tracking sensors, eye movements and/or behaviors (e.g., the eye movements 1146, the responses 1148) during the scenarios.

Referring back to FIG. 17A, the computer device 140 also evaluates (e.g., in step 1710) (e.g., using the evaluation/measurement module 1150) the monitored data for indicators of cognitive load and mental fatigue (e.g., the cognitive load and/or mental fatigue 1162). Referring next to FIG. 17D, in some embodiments, the computer device 140 evaluates the monitored data by assessing (e.g., in step 1720) blink rate, with a drop in rate signaling high cognitive load, measuring (e.g., in step 1722) fixation stability, with instability indicating difficulty in maintaining focus, and/or analyzing (e.g., in step 1724) saccadic movements, with longer saccades reflecting increased cognitive load.

Referring next to FIG. 17B, in some embodiments, the high-resolution VR headset has a visual fidelity of at least 60 pixels per degree (PPD) and/or a responsiveness with latency less than 20 ms.

Referring next to FIG. 17E, in some embodiments, the computer device 140 also progressively increases (e.g., in step 1726) difficulty and/or time constraints by gradually increasing the number of tasks and/or speed of stimuli, while decreasing time allowances for each task. In some embodiments, the computer device 140 also performs (e.g., in step 1728) an initial calibration to establish baseline cognitive and/or visual performance; and/or dynamically adjusts (e.g., in step 1730) task difficulty based on real-time performance metrics.

Referring next to FIG. 17F, in some embodiments, the computer device 140 also uses (e.g., in step 1732) one or more algorithms to evaluate cognitive performance by monitoring visual acuity, measuring reaction time, and/or analyzing error rates. In some embodiments, the computer device 140 monitors (e.g., in step 1734) visual acuity by tracking real-time task performance metrics in the VR environment. In some embodiments, the computer device 140 measures (e.g., in step 1734) reaction time by analyzing the time taken to respond to visual cues presented in the VR scenarios. In some embodiments, the computer device 140 analyzes (e.g., in step 1736) error rates by detecting patterns of cognitive overload based on mistakes made during the multitasking scenarios. In some embodiments, the computer device 140 also generates (e.g., in step 1738) a comprehensive report including cognitive load indicators, visual performance metrics, and/or error rates. In some embodiments, the comprehensive report includes (e.g., in step 1740) graphs, charts, and/or personalized recommendations for mitigating cognitive fatigue.

Referring next to FIG. 17G, in some embodiments, the computer device 140 also provides (e.g., in step 1740) personalized strategies for mitigating mental fatigue, including tailored breaks, task difficulty adjustments, and/or ergonomic suggestions based on individual cognitive capacity and/or task performance. In some embodiments, the computer device 140 also implements (e.g., in step 1742) safety and/or comfort measures including, for example: provides (e.g., in step 1744) visual and/or auditory cues for relaxation, offers (e.g., in step 1746) adjustable session lengths, presents (e.g., in step 1748) break reminders; and/or allows (e.g., in step 1750) for customizable difficulty levels. In some embodiments, the computer device 140 also reassesses (e.g., in step 1752) cognitive load after implementing mitigation strategies; and/or validates (e.g., in step 1754) the effectiveness of the strategies based on the reassessment. In some embodiments, the series of interactive multitasking scenarios include (e.g., in step 1756) one or more scenarios tailored to specific professional environments. In some embodiments, the computer device 140 also updates (e.g., in step 1758) the multitasking scenarios to reflect new research and/or workplace demands; and/or allows (e.g., in step 1760) for scenario adjustments to fit specific professional contexts.

Example details of various steps described herein are further described above in reference to FIGS. 12A-12H, according to some embodiments.

### VR Eye Strain Identification System

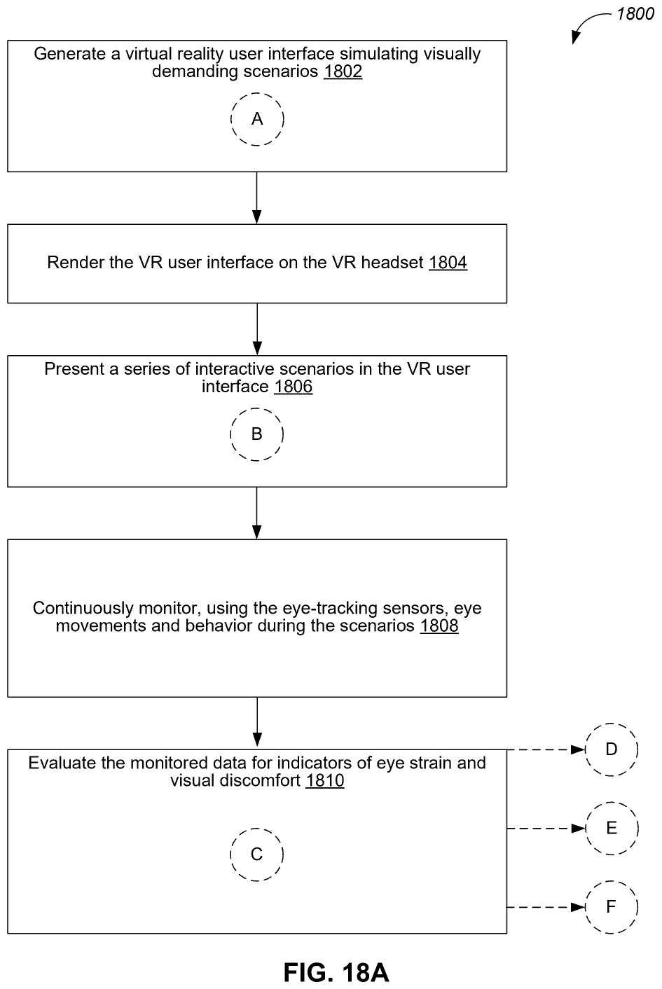

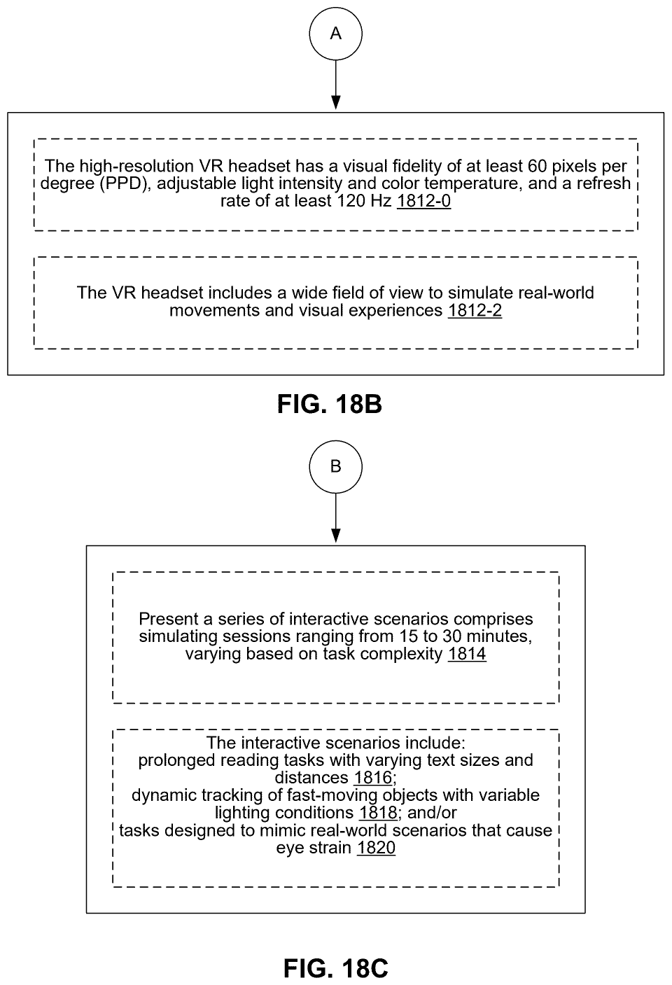

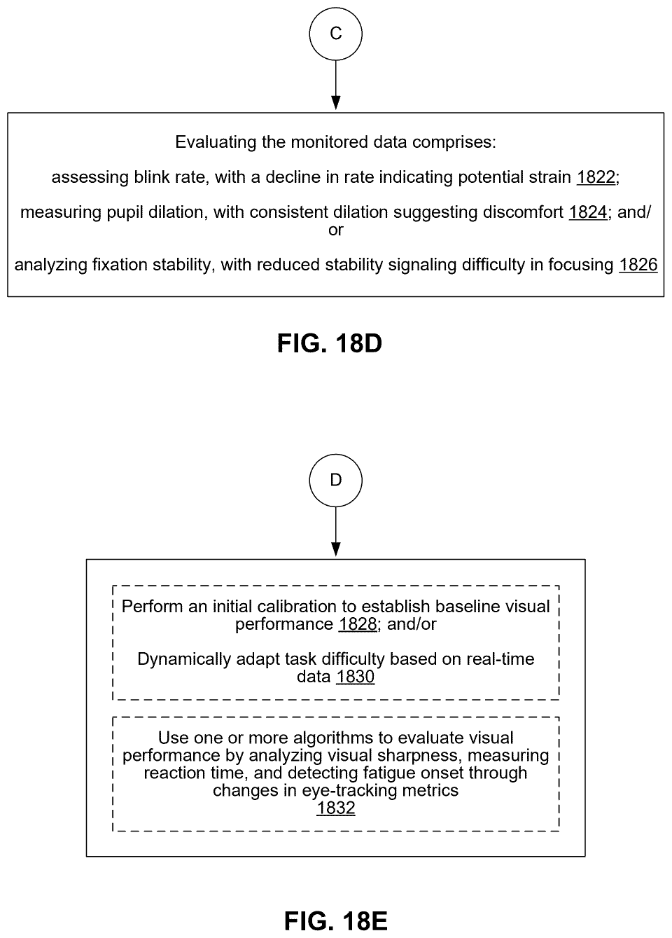

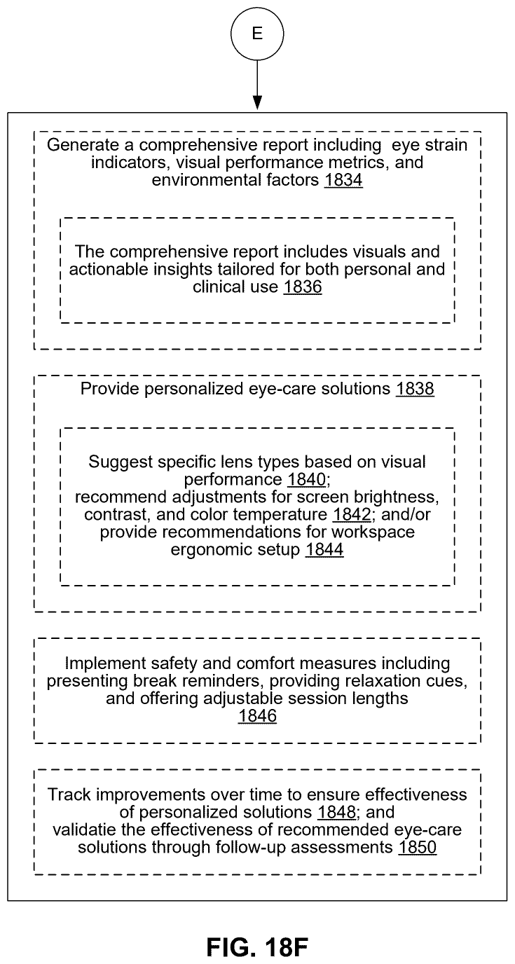

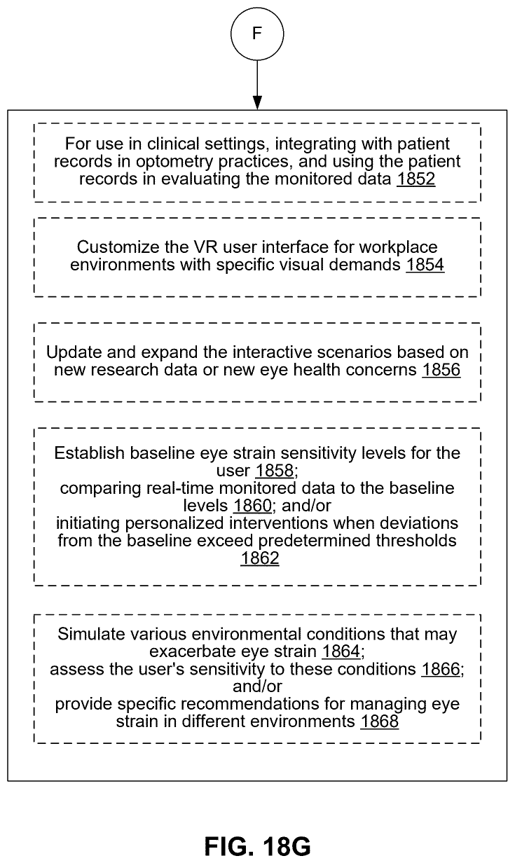

According to some embodiments, the vision test system 1100 described above is configured for evaluating visual discomfort in users with eye strain sensitivity. FIGS. 18A-18G show a flow diagram of an example process 1800 for evaluating visual discomfort in users with eye strain sensitivity, according to some embodiments. Traditional assessments include subjective questionnaires, Snellen charts, and manual testing of accommodation responses. These methods are less dynamic and do not offer real-time monitoring. Accordingly, the techniques described herein address at least some of these problems.

The computer device 140 (e.g., the computing device described above in reference to FIGS. 11A and 11B) generates (e.g., in step 1802) (e.g., using the UI module 1134) a VR user interface simulating visually demanding tasks (e.g., the environment 1136). Referring next to FIG. 18B, in some embodiments, (e.g., in step 1812-0) the high-resolution VR headset has a visual fidelity of at least 60 pixels per degree (PPD), adjustable light intensity and/or color temperature, and/or a refresh rate of at least 120 Hz. In some embodiments, (e.g., in step 1812-2) the VR headset includes a wide field of view to simulate real-world movements and/or visual experiences.

Referring back to FIG. 18A, the computer device 140 also renders (e.g., in step 1804) (e.g., using the rendering module 1138) the VR user interface on the VR headset (e.g., on the HMD 312A). Example details of the three-dimensional virtual environment and rendering the VR user interface are described above in reference to FIG. 12B, according to some embodiments.

The computer device 140 also presents (e.g., in step 1806) (e.g., using the simulation module 1140), in the VR user interface, a series of interactive scenarios (e.g., the scenarios 1142). In some embodiments, the computer device 140 presents (e.g., in step 1814) a series of interactive scenarios by simulating sessions ranging from 15 to 30 minutes, varying based on task complexity. In some embodiments, the interactive scenarios includes, (e.g., in step 1816) prolonged reading tasks with varying text sizes and/or distances, (e.g., in step 1818) dynamic tracking of fast-moving objects with variable lighting conditions, and/or (e.g., in step 1820) tasks designed to mimic real-world scenarios that cause eye strain.

Referring back to FIG. 18A, the computer device 140 also continuously monitors (e.g., in step 1808) (e.g., using the tracking module 1144), using the eye-tracking sensors, eye movements and/or behaviors (e.g., the eye movements 1146, the responses 1148) during the scenarios.

Referring back to FIG. 18A, the computer device 140 also evaluates (e.g., in step 1810) (e.g., using the evaluation/measurement module 1150) the monitored data for indicators of eye strain and visual discomfort (e.g., the eye strain and/or visual discomfort 1158). Referring next to FIG. 18D, in some embodiments, the computer device 140 evaluates the monitored data by: assessing (e.g., in step 1822) blink rate, with a decline in rate indicating potential strain, measuring (e.g., in step 1824) pupil dilation, with consistent dilation suggesting discomfort, and/or analyzing (e.g., in step 1826) fixation stability, with reduced stability signaling difficulty in focusing.

Referring next to FIG. 18E, in some embodiments, the computer device 140 also performs (e.g., in step 1828) an initial calibration to establish baseline visual performance, and/or dynamically adapts (e.g., in step 1830) task difficulty based on real-time data. In some embodiments, the computer device 140 also uses (e.g., in step) 1832 one or more algorithms to evaluate visual performance by analyzing visual sharpness, measuring reaction time, and/or detecting fatigue onset through changes in eye-tracking metrics.

Referring next to FIG. 18F, in some embodiments, the computer device 140 also generates (e.g., in step 1834) a comprehensive report including eye strain indicators, visual performance metrics, and/or environmental factors. In some embodiments, the comprehensive report includes (e.g., in step 1836) visuals and/or actionable insights tailored for both personal and/or clinical use. In some embodiments, the computer device 140 also provides (e.g., in step 1838) personalized eye-care solutions, including: The computer device 140 suggests (e.g., in step 1840) specific lens types based on visual performance, recommends (e.g., in step 1842) adjustments for screen brightness, contrast, and/or color temperature, and/or provides (e.g., in step 1844) recommendations for workspace ergonomic setup. In some embodiments, the computer device 140 also implements (e.g., in step 1846) safety and/or comfort measures including presenting break reminders, providing relaxation cues, and/or offering adjustable session lengths. In some embodiments, the computer device 140 also tracks (e.g., in step 1848) improvements over time to ensure effectiveness of personalized solutions; and/or validates (e.g., in step 1850) the effectiveness of recommended eye-care solutions through follow-up assessments.

Referring next to FIG. 18G, in some embodiments, for use in clinical settings, the computer device 140 also integrates (e.g., in step 1852) with patient records in optometry practices, and/or uses the patient records in evaluating the monitored data. In some embodiments, the computer device 140 also customizes (e.g., in step 1854) the VR user interface for workplace environments with specific visual demands. In some embodiments, the computer device 140 (e.g., in step 1856) updates and/or expands the interactive scenarios based on new research data and/or new eye health concerns. In some embodiments, the computer device 140 also establishes (e.g., in step 1858) baseline eye strain sensitivity levels for the user, compares (e.g., in step 1860) real-time monitored data to the baseline levels, and/or initiates (e.g., in step 1862) personalized interventions when deviations from the baseline exceed predetermined thresholds. In some embodiments, the computer device 140 also simulates (e.g., in step 1864) various environmental conditions that may exacerbate eye strain, assesses (e.g., in step 1866) the user's sensitivity to these conditions, and/or provides (e.g., in step 1868) specific recommendations for managing eye strain in different environments.

Example details of various steps described herein are further described above in reference to FIGS. 12A-12H, according to some embodiments.

### Example Vision Test Process

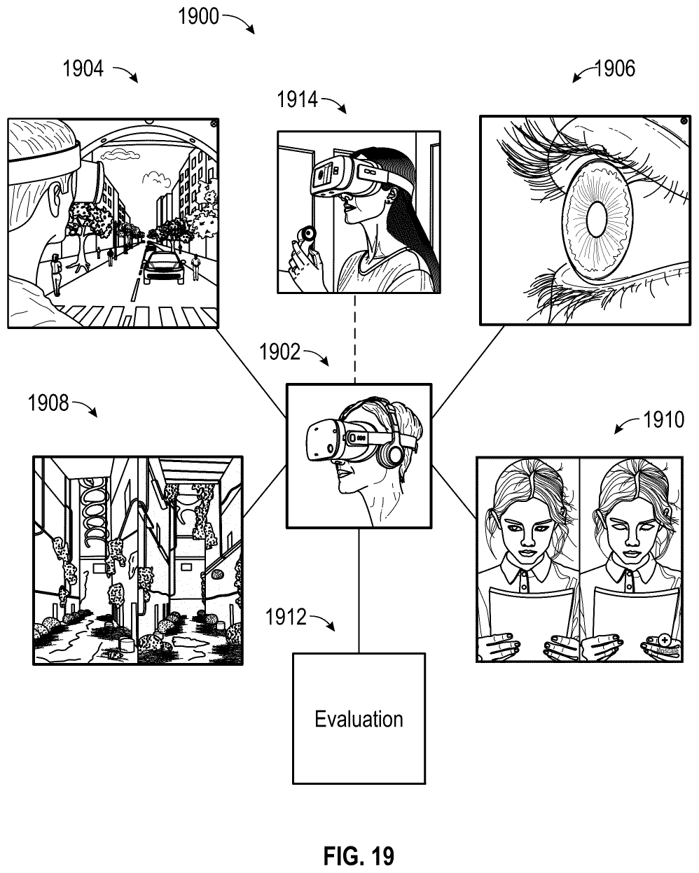

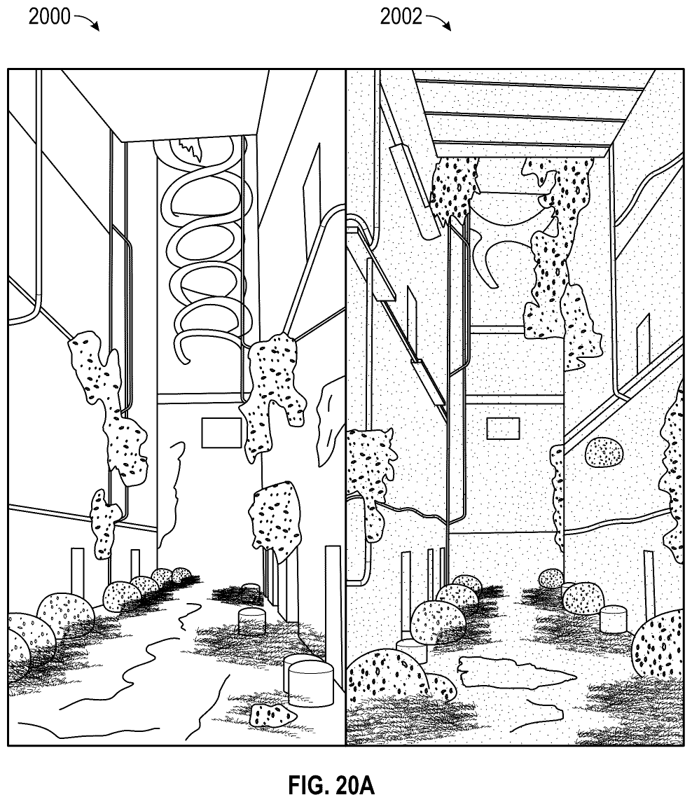

FIG. 19 is a schematic diagram showing an example vision test 1900, in accordance with some embodiments. The illustration 1902 shows a person wearing a VR headset (HMD). The VR headset may include eye-tracking cameras. As shown in the illustration 1904, the user's view through the HMD may show a three-dimensional virtual environment. An example of an environment is shown in the illustration 1910. The illustration 1906 shows a close-up of an eye that may be tracked by the eye-tracking cameras, which may track eye movements, such as saccades, fixations, and smooth pursuit. The illustration 1908 shows example scenarios that may be displayed in the HMD for evaluation response. Based on responses, the system may perform various evaluations (e.g., in step 1912). In some embodiments, the user may use a wearable device, an example of which is shown in the illustration 1914. Signals or output from the wearable device may be used in the evaluations.

### Example VR Eye Fatigue Monitoring and Adjustment System

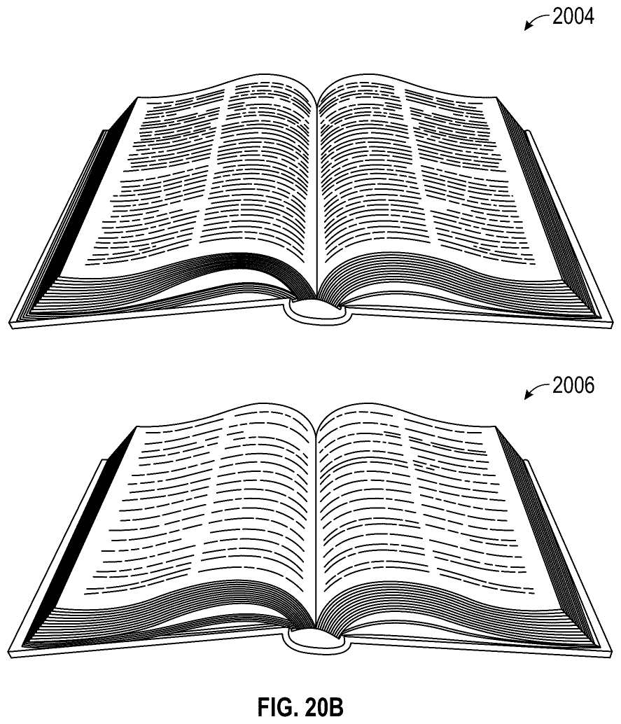

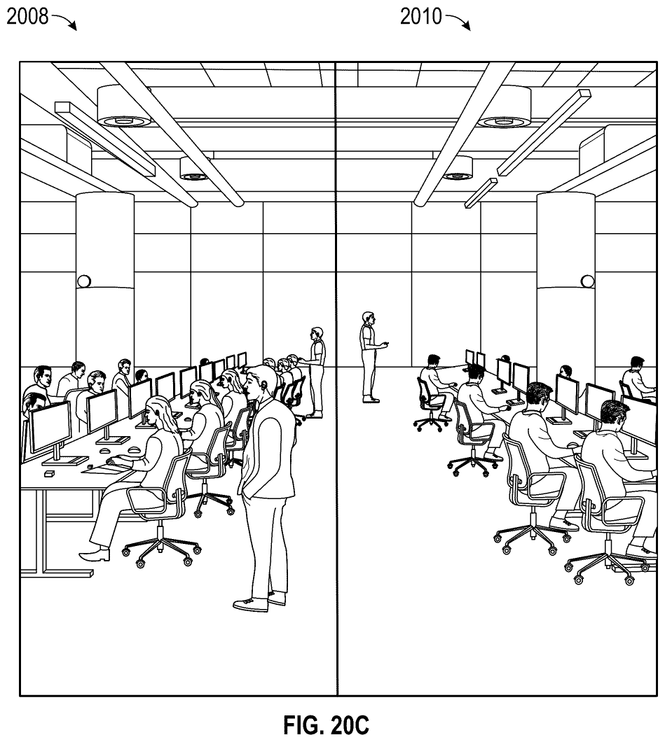

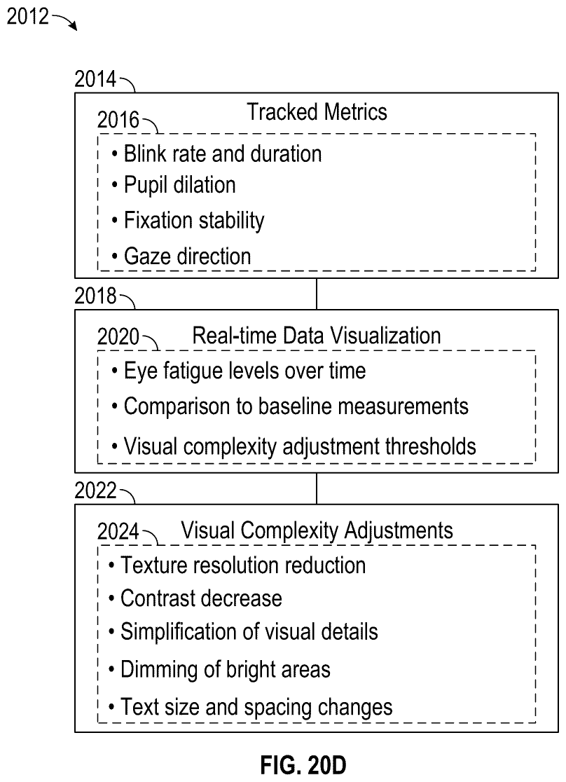

FIGS. 20A, 20B and 20C show illustrations of example visual scenarios for a VR eye fatigue monitoring and adjustment system, according to some embodiments. FIG. 20A show two panels, each panel showing a view of a maze in a video game. One panel 2000 has high visual complexity, the other panel 2002 has low visual complexity. FIG. 20B also shows two panels, each panel shows a view of an open textbook. One panel 2004 shows high visual complexity (shows a lot more lines) than the other panel 2006, which has low visual complexity (shows fewer lines). FIG. 20C shows two separate panels, each show a view of professional training centers with people and machines. First panel 2008 has high visual complexity (shows a lot more people and machines) than the other panel 2010, which has low visual complexity (shows fewer machines and people).

FIG. 20D is a block diagram of example components 2012 for a VR-based visual fatigue assessment and mitigation system, according to some embodiments. Some embodiments can include tracked metrics 2014, which may include, for example, blink rate and duration, pupil dilation, fixation stability, and/or gaze direction 2016. In some embodiments, real-time data visualization 2018 may include, for example, eye fatigue levels over time, comparison to baseline measurements, and/or visual complexity adjustment thresholds 2020. Some embodiments can include visual complexity adjustments 2022, which may include, for example, texture resolution reduction, contrast decrease, simplification of visual details, dimming of bright areas, and/or text size and spacing changes 2024.

### Example VR Real-Time Visual Health Monitoring System for Extended Use

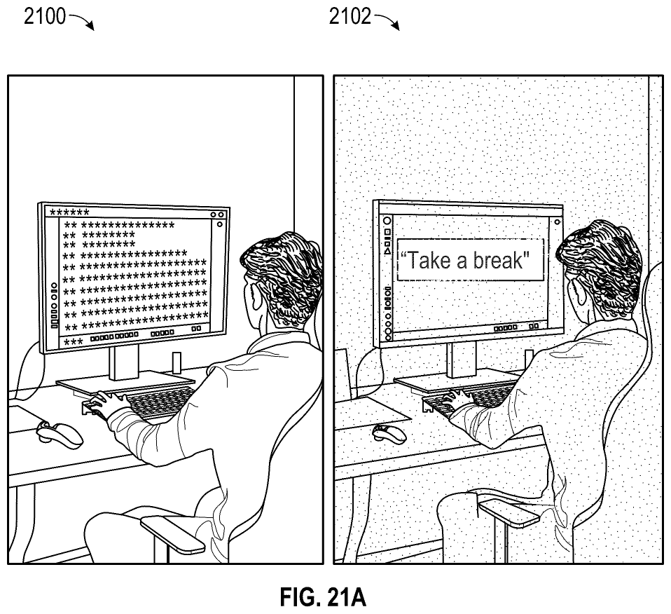

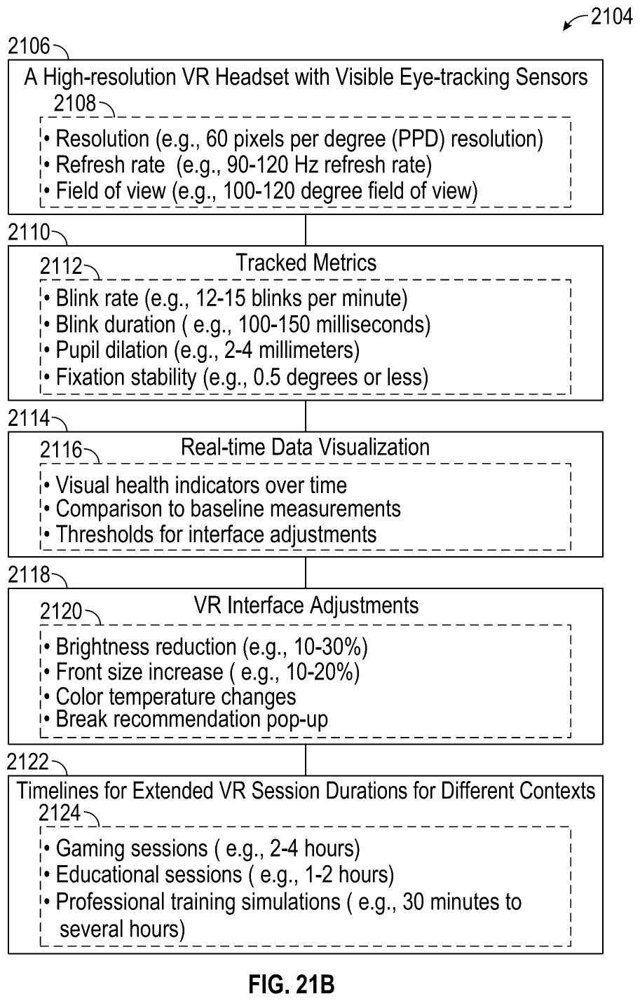

FIG. 21A shows illustrations of example scenarios for a VR real-time visual health monitoring system for extended use, according to some embodiments. The illustration shows two panels, each showing views of video gaming sessions on a screen viewed by a user. One panel 2100 shows bright light, text with large fonts, rich colors. Second panel 2102 shows dim light, text with smaller fonts, dull colors. A popup on top of the second panel says, “Take a break,” which is an example of a prompt/alert, according to some embodiments.

FIG. 21B is a block diagram of example components 2104 for a VR real-time visual health monitoring system for extended use, according to some embodiments. Some embodiments can include a high-resolution VR headset with visible eye-tracking sensors 2106, which may feature, for example, 60 pixels per degree (PPD) resolution, 90-120 Hz refresh rate, and/or 100-120-degree field of view 2108. Some embodiments can include tracked metrics 2110, which may measure, for example, blink rate (e.g., 12-15 blinks per minute), blink duration (e.g., 100-150 milliseconds), pupil dilation (e.g., 2-4 millimeters), and/or fixation stability (e.g., 0.5 degrees or less) 2112. In some embodiments, real-time data visualization 2114 may include, for example, visual health indicators over time, comparison to baseline measurements, and thresholds for interface adjustments 2116. Some embodiments can include VR interface adjustments 2118, which may encompass, for example, brightness reduction (e.g., 10-30%), font size increase (e.g., 10-20%), color temperature changes, and/or break recommendation pop-ups 2120. The system may also include timelines for extended VR session durations for different contexts 2122, such as gaming sessions (e.g., 2-4 hours), educational sessions (e.g., 1-2 hours), and/or professional training simulations (e.g., 30 minutes to several hours) 2124.

### Example VR-Based Vision Testing and Eye Health Monitoring System

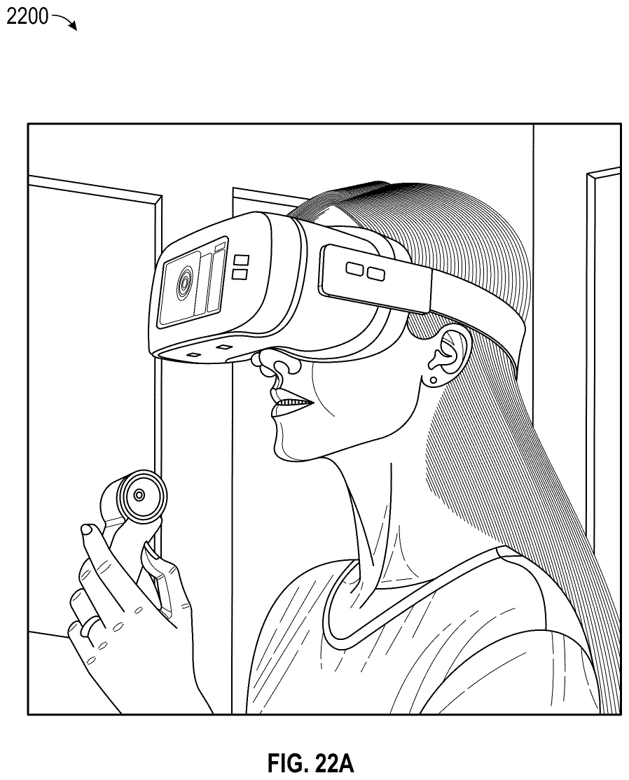

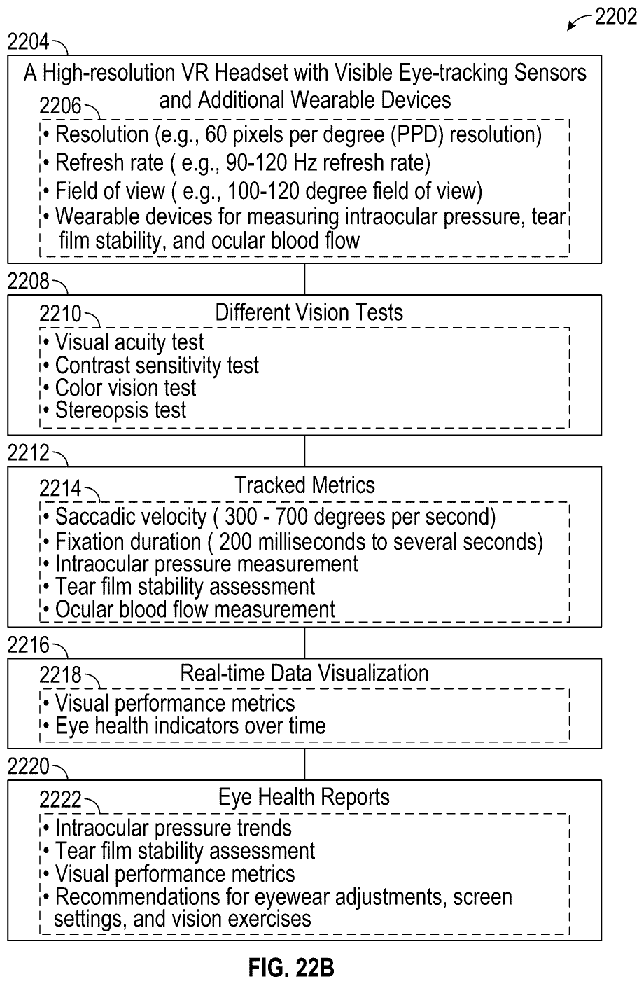

FIG. 22A shows an example view 2200 of a person wearing (i) a high-resolution VR headset with eye-tracking sensors, (ii) contact lens sensor, and (iii) handheld device, according to some embodiments. The handheld device may be used instead of, or in addition to, the eye-tracking sensors and/or the contact lens sensor (worn by the user), for VR-based vision testing and eye health monitoring, according to some embodiments.

FIG. 22B is a block diagram of example components 2202 for a VR-based vision testing and eye health monitoring system, according to some embodiments. Some embodiments can include a high-resolution VR headset with eye-tracking sensors and additional wearable devices 2204, which may feature, for example, 60 pixels per degree (PPD) resolution, 90-120 Hz refresh rate, 100-120 degree field of view, and/or wearable devices for measuring intraocular pressure, tear film stability, and/or ocular blood flow 2206. Some embodiments can include different vision tests 2208, such as visual acuity test, contrast sensitivity test, color vision test, and stereopsis test 2210. In some embodiments, tracked metrics 2212 may include, for example, saccadic velocity (e.g., 300-700 degrees per second), fixation duration (e.g., 200 milliseconds to several seconds), intraocular pressure measurement, tear film stability assessment, and/or ocular blood flow measurement 2214. Some embodiments can include real-time data visualization 2216, which may encompass, for example, visual performance metrics, eye health indicators over time, and/or comparison to baseline measurements and clinical thresholds 2218. The system may also include eye health reports 2220, which may include, for example, intraocular pressure trends, tear film stability assessment, visual performance metrics, and/or recommendations for eyewear adjustments, screen settings, and/or vision exercises 2222.

### Example VR Eye Strain Identification System Through Prolonged Engagement

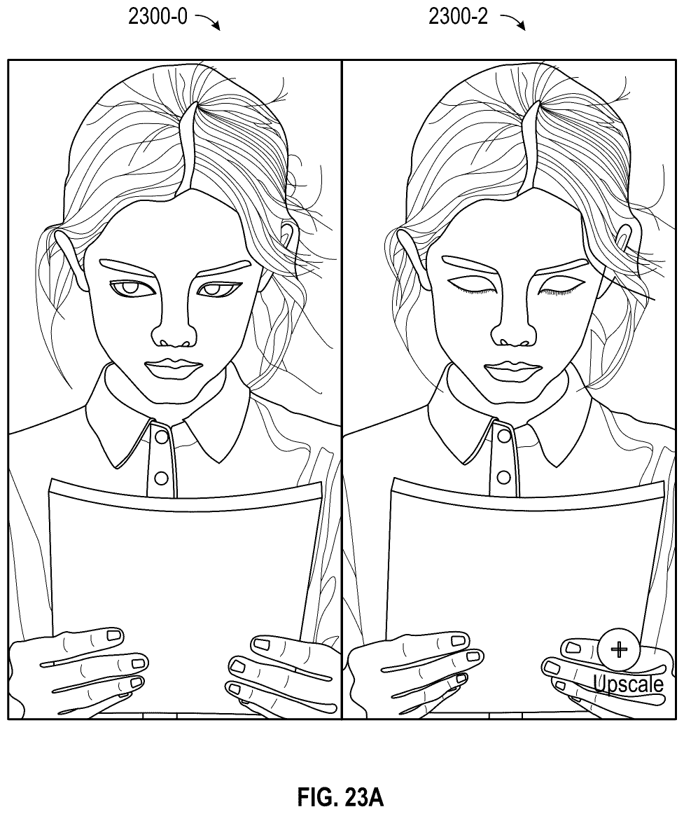

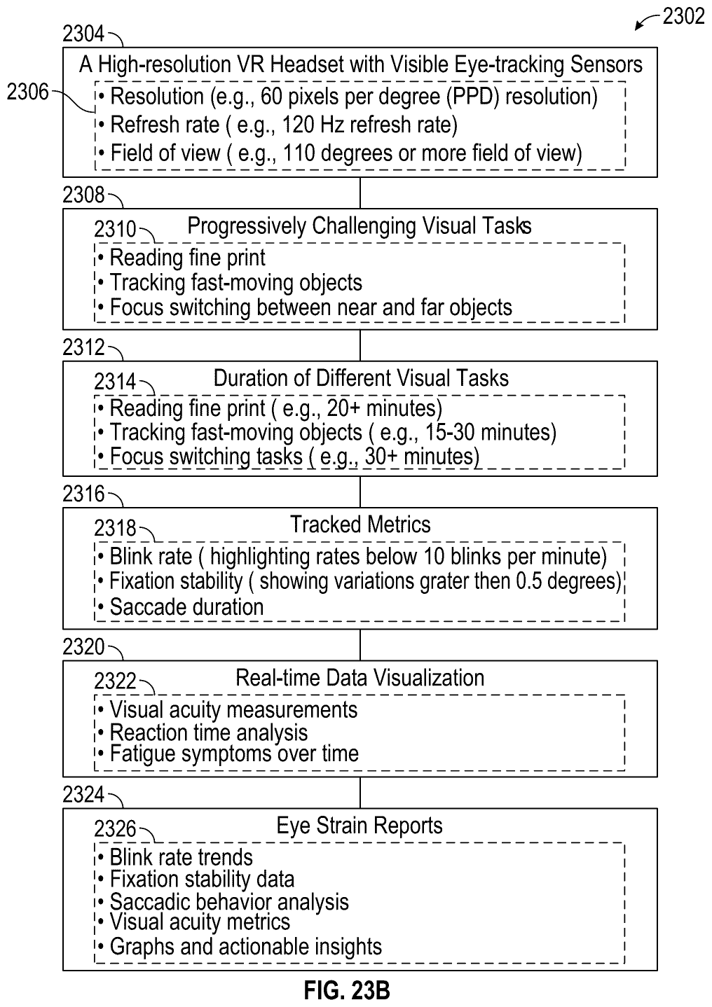

FIG. 23A shows illustrations of example scenarios for a VR eye strain identification system through prolonged engagement, according to some embodiments. The figure shows two panels, each showing a view of a person reading fine print. The panel 2300-0 shows the person with open eyes, and the panel 2300-2 show the person with slightly closing eyes, which may indicate eye strain.

FIG. 23B is a block diagram of example components 2302 for a VR-based eye strain identification system, according to some embodiments. Some embodiments can include a high-resolution VR headset with visible eye-tracking sensors 2304, which may feature, for example, 60 pixels per degree (PPD) resolution, 120 Hz refresh rate, and/or 110 degrees or more field of view 2306. Some embodiments can include progressively challenging visual tasks 2308, which may include, for example, reading fine print, tracking fast-moving objects, and/or focus switching between near and far objects 2310. The system may also incorporate duration of different visual tasks 2312, which may include, for example, reading fine print (e.g., 20+ minutes), tracking fast-moving objects (e.g., 15-30 minutes), and/or focus switching tasks (e.g., 30+ minutes) 2314. In some embodiments, tracked metrics 2316 may include blink rate (e.g., highlighting rates below 10 blinks per minute), fixation stability (e.g., showing variations greater than 0.5 degrees), and/or saccade duration 2318. Some embodiments can include real-time data visualization 2320, which may encompass, for example, visual acuity measurements, reaction time analysis, and/or fatigue symptoms over time 2322. The system may also generate eye strain reports 2324, which may include, for example, blink rate trends, fixation stability data, saccadic behavior analysis, visual acuity metrics, and/or graphs with actionable insights 2326.

### Example VR Blue Light Sensitivity Evaluation System for Digital Device Use

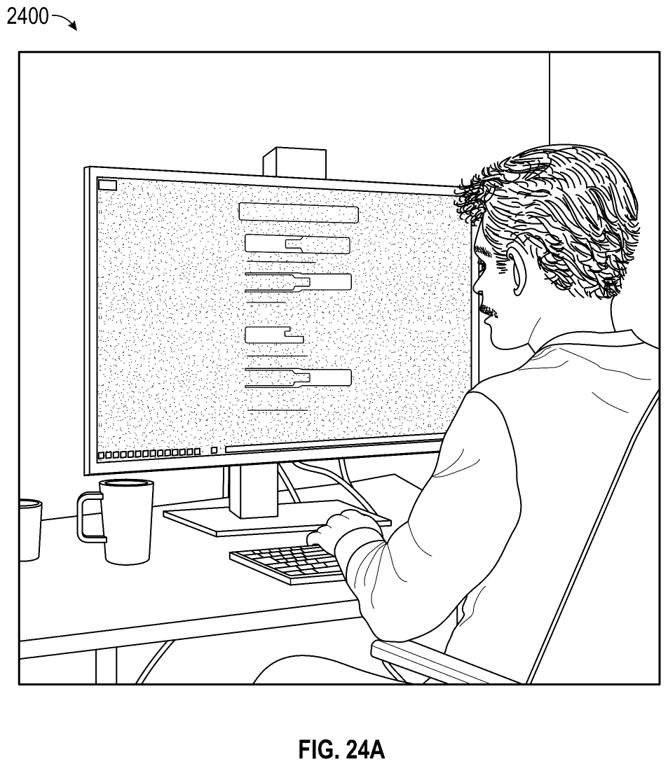

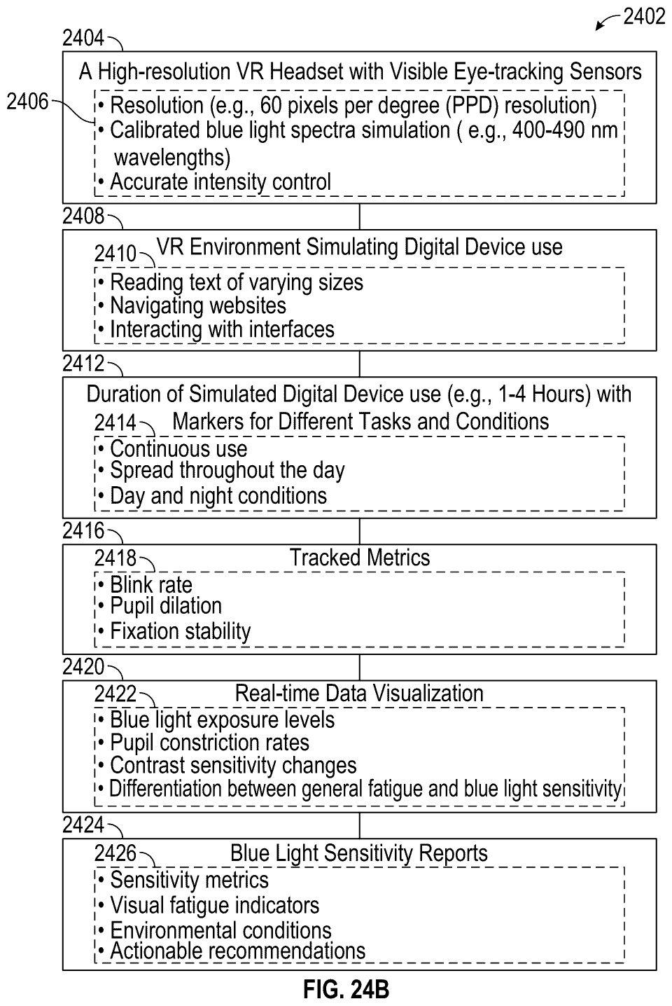

FIG. 24A shows an example view 2300 of a person navigating websites on a screen that shows a lot of blue light, for a VR-based blue light sensitivity evaluation system for digital device use, according to some embodiments.

FIG. 24B is a block diagram of example components 2402 for a VR-based blue light sensitivity assessment system, according to some embodiments. Some embodiments can include a high-resolution VR headset with eye-tracking sensors 2404, which may feature, for example, 60 pixels per degree (PPD) resolution, calibrated blue light spectra simulation (e.g., 400-490 nm wavelengths), and/or accurate intensity control 2406. Some embodiments can include a VR environment 2408 simulating digital device use, which may include, for example, reading text of varying sizes, navigating websites, and/or interacting with interfaces 2410. The system may incorporate duration of simulated digital device use (e.g., 1-4 hours) with markers for different tasks and conditions 2412, which may include, for example, continuous use, spread throughout the day, and/or day and night conditions 2414. In some embodiments, tracked metrics 2416 may include, for example, blink rate, pupil dilation, and/or fixation stability 2418. Some embodiments can include real-time data visualization 2420, which may encompass, for example, blue light exposure levels, pupil constriction rates, contrast sensitivity changes, and/or differentiation between general fatigue and blue light sensitivity 2422. The system may also generate blue light sensitivity reports 2424, which may include, for example, sensitivity metrics, visual fatigue indicators, environmental conditions, and/or actionable recommendations 2426.

### Example VR-Based Cognitive Load and Mental Fatigue Testing System

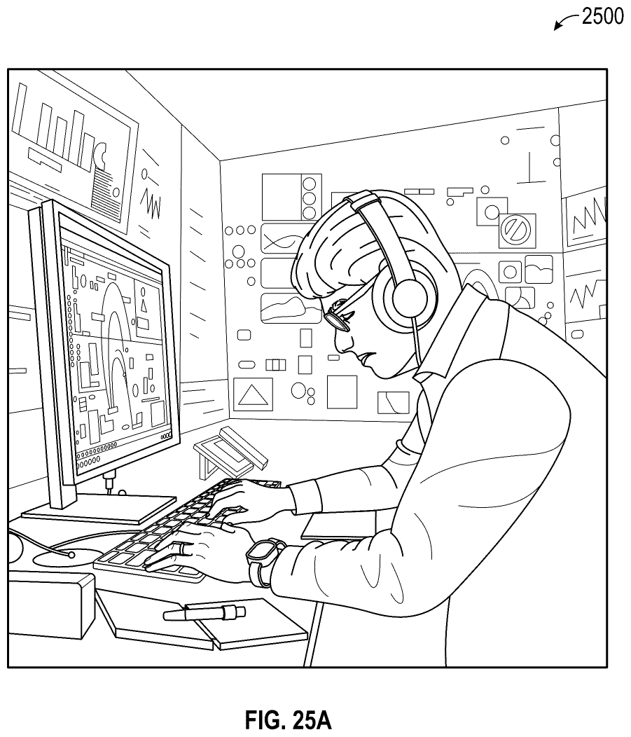

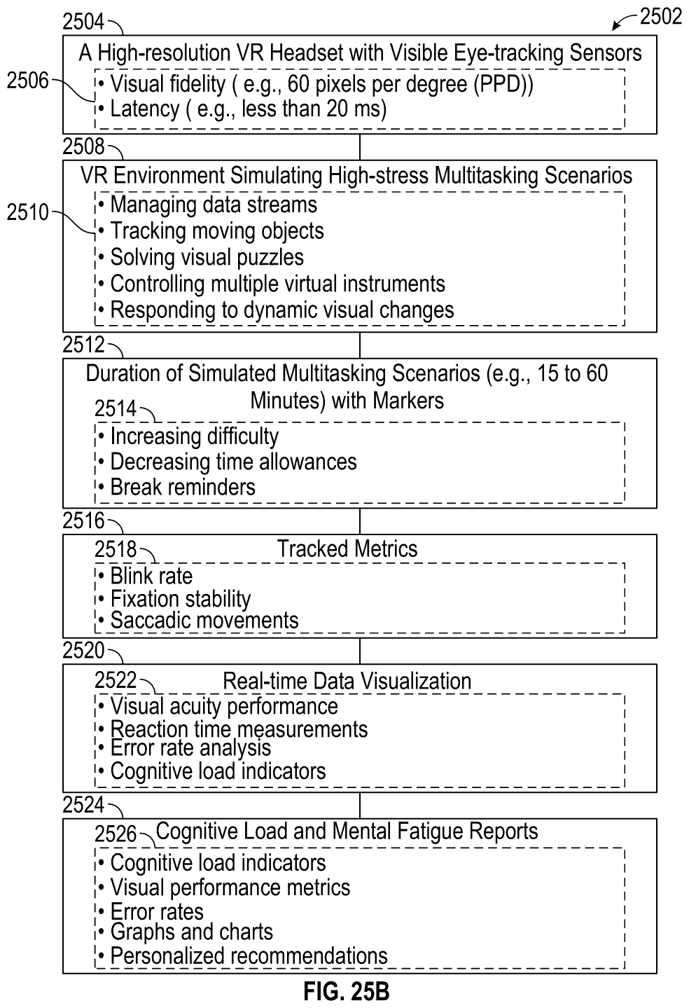

FIG. 25A shows an example view 2500 of a person engaged in a high-stress multitasking scenario involving solving visual puzzles, for a VR-based cognitive load and mental fatigue testing system, according to some embodiments.

FIG. 25B is a block diagram of example components 2502 for a VR-based cognitive load and mental fatigue assessment system, according to some embodiments. Some embodiments can include a high-resolution VR headset with visible eye-tracking sensors 2504, which may include, for example, visual fidelity of 60 pixels per degree (PPD), and/or latency of less than 20 ms 2506. Some embodiments can include a VR environment 2508 simulating high-stress multitasking scenarios, which may include, for example, managing data streams, tracking moving objects, solving visual puzzles, controlling multiple virtual instruments, and/or responding to dynamic visual changes 2510. The system may incorporate duration of simulated multitasking scenarios (e.g., 15 to 60 minutes) with markers 2512, which may include, for example, increasing difficulty, decreasing time allowances, and/or break reminders 2514. In some embodiments, tracked metrics 2516 may include, for example, blink rate, fixation stability, and/or saccadic movements 2518. Some embodiments can include real-time data visualization 2520, which may encompass, for example, visual acuity performance, reaction time measurements, error rate analysis, and/or cognitive load indicators 2522. The system may also generate cognitive load and mental fatigue reports 2524, which may include, for example, cognitive load indicators, visual performance metrics, error rates, graphs and charts, and/or personalized recommendations 2526.

### Example VR-Based Eye Strain Identification System for Visual Discomfort Evaluation

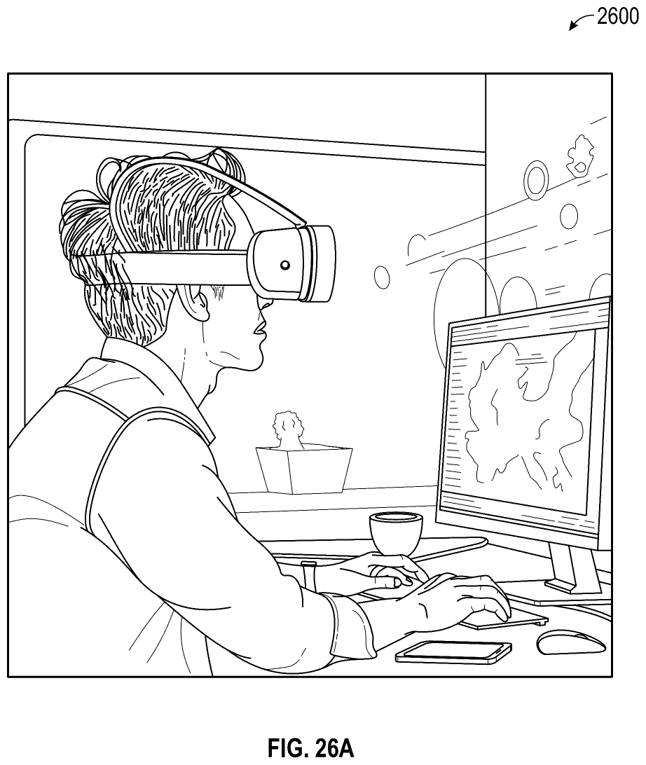

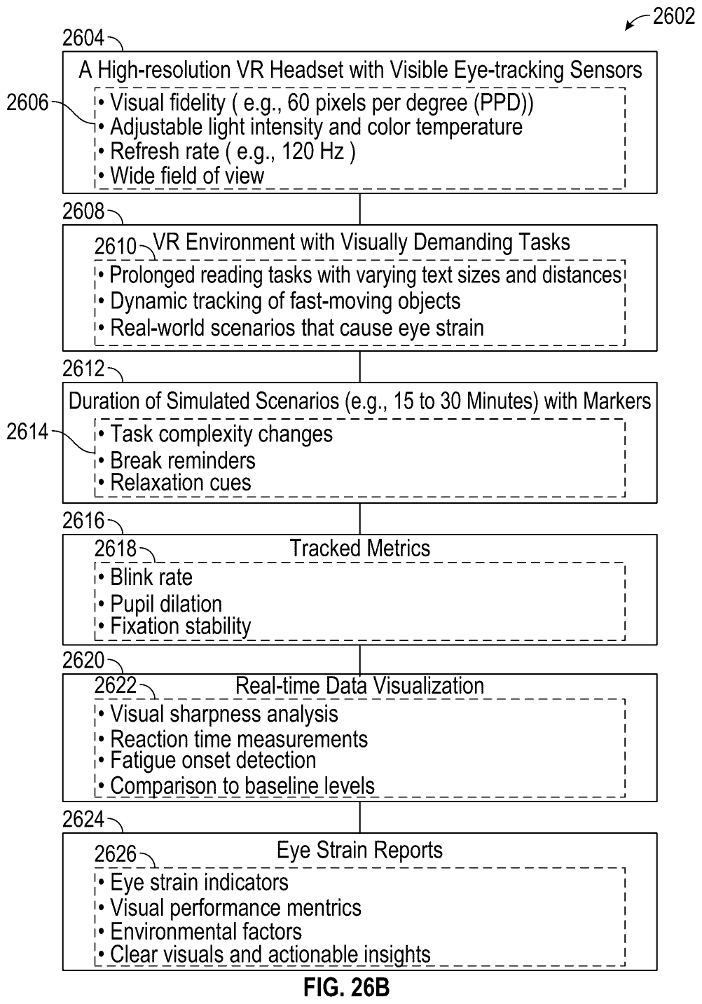

FIG. 26A shows an example view 2600 of a person engaged in a visually demanding task for dynamic tracking of fast-moving objects, for a VR-based eye strain identification system for visual discomfort evaluation, according to some embodiments.

FIG. 26B is a block diagram of example components 2602 for a VR-based eye strain assessment and monitoring system, according to some embodiments. Some embodiments can include a high-resolution VR headset with visible eye-tracking sensors 2604, which may feature, for example, visual fidelity of 60 pixels per degree (PPD), adjustable light intensity and color temperature, a refresh rate of 120 Hz, and a wide field of view 2606. Some embodiments can include a VR environment with visually demanding tasks 2608, which may include, for example, prolonged reading tasks with varying text sizes and distances, dynamic tracking of fast-moving objects, and/or real-world scenarios that cause eye strain 2610. The system may incorporate duration of simulated scenarios (e.g., 15 to 30 minutes) with markers 2612, which may include, for example, task complexity changes, break reminders, and/or relaxation cues 2614. In some embodiments, tracked metrics 2616 may include, for example, blink rate, pupil dilation, and/or fixation stability 2618. Some embodiments can include real-time data visualization 2620, which may encompass, for example, visual sharpness analysis, reaction time measurements, fatigue onset detection, and/or comparison to baseline levels 2622. The system may also generate eye strain reports 2624, which may include, for example, eye strain indicators, visual performance metrics, environmental factors, and/or clear visuals with actionable insights 2626.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method of implementing a virtual reality (VR) system for implementing a virtual reality (VR) system that adjusts visual complexity based on real-time eye fatigue monitoring, comprising: at an electronic device including a head-mounted display and eye-tracking sensors: generating a VR user interface corresponding to a three-dimensional virtual environment; rendering the VR user interface on the head-mounted display; continuously monitoring, using the eye-tracking sensors, user eye movements and behavior; detecting eye fatigue based on the user eye movements and behavior; and dynamically adjusting the visual complexity of the VR user interface based on the detected eye fatigue.

Clause 2. The method of Clause 1, wherein monitoring user eye movements and behavior comprises tracking blink rate, blink duration, pupil dilation, and fixation stability.

Clause 3. The method of any of Clauses 1 or 2, wherein dynamically adjusting the visual complexity comprises reducing texture resolution, decreasing contrast, simplifying visual details, and dimming bright areas.

Clause 4. The method of any of Clauses 1-3, further comprising adjusting task complexity by reducing the number of simultaneous visual elements based on detected eye fatigue levels.

Clause 5. The method of any of Clauses 1-4, wherein the method is applied in different contexts including education, gaming, and professional training, with context-specific adjustments based on fatigue indicators.

Clause 6. The method of Clause 5, wherein in a virtual classroom setting, adjustments comprise reducing text density, increasing line spacing, and simplifying background visuals.

Clause 7. The method of Clause 5, wherein in a gaming environment, adjustments comprise lowering texture resolution, reducing brightness and dynamic lighting effects, and smoothing or slowing down motion effects.

Clause 8. The method of any of Clauses 1-7, wherein detecting eye fatigue comprises detecting signs of visual fatigue based on changes in eye-tracking metrics, wherein increased blink rate, longer blinks, prolonged pupil dilation or reduced fixation stability indicate eye fatigue.

Clause 9. The method of any of Clauses 1-8, using one or more algorithms for pattern recognition to detect signs of fatigue and visual scene simplification to gradually reduce visual complexity.

Clause 10. The method of any of Clauses 1-9, further comprising generating a comprehensive report on visual endurance, including insights on fatigue progression, optimal screen time recommendations, and personalized adjustments.

Clause 11. The method of any of Clauses 1-10, wherein the eye-tracking technology comprises infrared cameras with a sampling rate of 200 Hz or higher and sub-degree precision in tracking gaze direction with latency under 10 ms.

Clause 12. The method of any of Clauses 1-11, wherein in interactive VR scenarios, adjusting visual complexity comprises modifying text size, reading speed, and visual complexity of diagrams in educational modules, and modulating difficulty levels, NPC density, and environmental effects in gaming environments.

Clause 13. The method of any of Clauses 1-12, further comprising calibrating and validating the system using a control group to establish baseline measurements of eye movements and visual performance.

Clause 14. The method of any of Clauses 1-13, further comprising generating recommendations for optimal VR usage durations, including session limits, specific break intervals, and visual settings tailored to the user's endurance profile.

Clause 15. The method of any of Clauses 1-14, wherein monitoring user eye movements and behavior and adjusting visual complexity occur in real-time with a latency of less than 100 milliseconds.

Clause 16. The method of any of Clauses 1-15, further comprising: establishing baseline eye fatigue levels for the user; comparing real-time eye tracking data to the baseline levels; and initiating visual complexity adjustments when deviations from the baseline exceed predetermined thresholds.

Clause 17. The method of any of Clauses 1-16, wherein adjusting visual complexity is performed gradually to avoid abrupt changes that may disrupt user experience.

Clause 18. The method of any of Clauses 1-17, further comprising: allowing user input to fine-tune the sensitivity of fatigue detection and the degree of visual adjustments; storing user preferences for future VR sessions; and adapting the system's response to eye fatigue based on accumulated user data over multiple sessions.

Clause 19. A method of implementing a virtual reality (VR) system for real-time visual health monitoring during extended use, comprising: at an electronic device including a head-mounted display and eye-tracking sensors: generating a VR user interface corresponding to a three-dimensional virtual environment; rendering the VR user interface on the VR headset; continuously monitoring, using the eye-tracking sensors, user eye movements and behavior during extended VR sessions; and dynamically adjusting the VR user interface based on detected visual health indicators.

Clause 20. The method of any of Clauses 19, wherein the high-resolution VR headset has a resolution of at least 60 pixels per degree (PPD), a refresh rate of 90-120 Hz, and a field of view of 100-120 degrees, and wherein the eye-tracking sensors have an accuracy of 0.1-degree precision and a latency of less than 10 milliseconds.

Clause 21. The method of any of Clauses 19 or 20, wherein monitoring user eye movements and behavior comprises tracking blink rate, blink duration, pupil dilation, and fixation stability.

Clause 22. The method of Clause 21, wherein tracking blink rate comprises measuring the number of blinks per minute, with 12-15 blinks per minute considered normal at rest.

Clause 23. The method of Clause 21, wherein tracking blink duration comprises measuring the length of each blink, with 100-150 milliseconds considered normal.

Clause 24. The method of Clause 21, wherein tracking pupil dilation comprises measuring pupil size, with 2-4 millimeters considered normal.

Clause 25. The method of Clause 21, wherein tracking fixation stability comprises measuring eye movement during fixation, with 0.5 degrees or less considered stable.

Clause 26. The method of any of Clauses 19-25, wherein dynamically adjusting the VR user interface comprises providing break recommendations based on cumulative strain metrics.

Clause 27. The method of any of Clauses 19-26, wherein dynamically adjusting the VR user interface comprises modifying display settings including brightness, contrast, or color temperature.

Clause 28. The method of Clause 27, wherein modifying display settings comprises reducing brightness by 10-30% or increasing font size by 10-20% during prolonged reading tasks.

Clause 29. The method of any of Clauses 19-28, further comprising using machine learning algorithms to detect patterns of fatigue based on historical data.

Clause 30. The method of any of Clauses 19-29, further comprising using predictive models to anticipate when fatigue will likely occur and preemptively adjust visual settings.

Clause 31. The method of any of Clauses 19-30, further comprising generating a visual health report including visual strain indicators over time, recommended adjustments, and long-term trends.

Clause 32. The method of any of Clauses 19-31, wherein detecting visual health indicators comprises tracking blink rate, blink duration, pupil dilation and fixation stability, wherein increased blink rate and duration indicates fatigue, diminished fixation stability indicates strain, and persistent pupil dilation indicates excessive cognitive load or discomfort.

Clause 33. The method of any of Clauses 19-32, further comprising providing a user interface for real-time feedback and recommendations related to visual health.

Clause 34. The method of any of Clauses 19-33, further comprising calibrating the system using a control group of 20-50 individuals with diverse age and visual profiles.

Clause 35. The method of any of Clauses 19-34, wherein the extended VR sessions comprise gaming sessions lasting 2-4 hours, educational sessions lasting 1-2 hours, or professional training simulations lasting 30 minutes to several hours.

Clause 36. The method of any of Clauses 19-35, further comprising: establishing baseline visual health metrics for the user; comparing real-time eye tracking data to the baseline metrics; and initiating visual interface adjustments when deviations from the baseline exceed predetermined thresholds.

Clause 37. A method of implementing a virtual reality (VR) system for vision testing and eye health monitoring, comprising: at an electronic device including a high-resolution VR headset with eye-tracking sensors and wearable devices for measuring intraocular pressure, tear film stability, and ocular blood flow: generating a VR user interface corresponding to a three-dimensional virtual environment; rendering the VR user interface on the VR headset; conducting a series of vision tests in the VR environment; continuously monitoring, using the eye-tracking sensors and wearable devices, eye movements and vitals during the vision tests; and evaluating the monitored data for visual performance and eye health assessment.

Clause 38. The method of any of Clauses 37, wherein the high-resolution VR headset has a resolution of at least 60 pixels per degree (PPD), a refresh rate of 90-120 Hz, and a field of view of 100-120 degrees.

Clause 39. The method of any of Clauses 37 or 38, wherein the wearable devices measure intraocular pressure with an accuracy of ±1 mmHg, tear film stability by assessing break-up time, and ocular blood flow using near-infrared spectroscopy with an accuracy of ±5%.

Clause 40. The method of any of Clauses 37-39, wherein conducting a series of vision tests comprises performing tests for visual acuity, contrast sensitivity, color vision, and stereopsis.

Clause 41. The method of Clause 40, wherein the series of vision tests typically lasts 15-30 minutes depending on the test battery.

Clause 42. The method of any of Clauses 37-41, wherein monitoring eye movements comprises tracking saccadic velocity and fixation duration.

Clause 43. The method of any of Clause 42, wherein tracking saccadic velocity comprises measuring eye movement speeds typically ranging from 300-700 degrees per second.

Clause 44. The method of Clause 42, wherein tracking fixation duration comprises measuring eye focus durations ranging from 200 milliseconds to several seconds, depending on task complexity.

Clause 45. The method of any of Clauses 37-44, wherein evaluating the monitored data comprises correlating intraocular pressure, tear film stability, and ocular blood flow with visual performance metrics.

Clause 46. The method of Clause 45, wherein correlating comprises associating elevated intraocular pressure with decreased visual field sensitivity, unstable tear film with fluctuating vision quality, and reduced ocular blood flow with potential issues in visual acuity under stress.

Clause 47. The method of any of Clauses 37-46, further comprising using algorithms to process data related to visual clarity, reaction time, and stability of vision.

Clause 48. The method of any of Clauses 37-47, further comprising generating a detailed report including insights on intraocular pressure trends, tear film stability, visual performance metrics, and recommendations for eyewear adjustments, screen settings, and vision exercises.

Clause 49. The method of any of Clauses 37-48, further comprising comparing monitored data against established clinical thresholds to flag potential issues, such as intraocular pressure exceeding 21 mmHg for glaucoma risk.

Clause 50. The method of any of Clauses 37-49, wherein the eye-tracking sensors have an accuracy within 0.1 mm of eye movement and a latency of less than 10 milliseconds.

Clause 51. The method of any of Clauses 37-50, further comprising calibrating the system using a diverse control group of 30-50 individuals with a range of visual conditions.

Clause 52. The method of any of Clauses 37-51, further comprising encrypting all visual health data at rest and in transit and ensuring compliance with HIPAA and GDPR standards for handling health data.

Clause 53. The method of any of Clauses 37-52, wherein the method is adaptable for use in both clinical and personal eye care settings, with clinical use involving more detailed reporting and integration with EMR systems, and personal use involving a simpler interface with recommendations tailored for non-clinical use.

Clause 54. The method of any of Clauses 37-53, further comprising: establishing baseline visual performance and eye health metrics for the user; comparing real-time monitored data to the baseline metrics; and providing personalized recommendations when deviations from the baseline exceed predetermined thresholds.

Clause 55. A method of implementing a virtual reality (VR) system for identifying potential eye strain issues through prolonged engagement, comprising: at an electronic device including a high-resolution VR headset with eye-tracking sensors: generating a VR user interface corresponding to a three-dimensional virtual environment; rendering the VR user interface on the VR headset; presenting a series of progressively challenging visual tasks in the VR environment; continuously monitoring eye movements and behavior during the visual tasks; and evaluating the monitored data for indicators of eye strain.

Clause 56. The method of any of Clauses 55, wherein the high-resolution VR headset has a resolution of at least 60 pixels per degree (PPD), a refresh rate of 120 Hz, and a field of view of 110 degrees or more.

Clause 57. The method of any of Clauses 55 or 56, wherein the series of visual tasks includes reading fine print for periods exceeding 20 minutes, tracking fast-moving objects for 15-30 minutes, and focus switching tasks for periods over 30 minutes.

Clause 58. The method of any of Clauses 55-57, wherein the progressively challenging visual tasks comprise reading documents of varying font sizes, tracking fast-moving objects, and switching focus between near and far objects.

Clause 59. The method of Clause 58, wherein the tasks start with low complexity and gradually introduce more elements or faster movement to challenge the user.

Clause 60. The method of any of Clauses 55-59, wherein evaluating the monitored data comprises: assessing blink rate, with rates below 10 blinks per minute indicating potential fatigue; measuring fixation stability, with variations greater than 0.5 degrees suggesting strain; and analyzing saccade duration, with prolonged saccades indicating increased cognitive load.

Clause 61. The method of any of Clauses 55-60, further comprising: performing an initial calibration to establish baseline visual performance for the user; and dynamically adapting the difficulty of visual tasks in real-time based on user performance and strain indicators.

Clause 62. The method of any of Clauses 55-61, further comprising using one or more algorithms to assess visual acuity, reaction time, and fatigue symptoms.

Clause 63. The method of Clause 62, wherein assessing visual acuity comprises conducting sharpness and clarity tests in the VR environment.

Clause 64. The method of Clause 62, wherein measuring reaction time comprises analyzing how quickly users respond to visual stimuli presented in the VR environment.

Clause 65. The method of Clause 62, wherein detecting fatigue symptoms comprises analyzing changes in blink rate and saccadic patterns over time.

Clause 66. The method of any of Clauses 55-65, further comprising generating a comprehensive report including blink rate trends, fixation stability data, saccadic behavior analysis, and visual acuity metrics.

Clause 67. The method of Clause 66, wherein the comprehensive report includes graphs and actionable insights for both users and clinicians.

Clause 68. The method of any of Clauses 55-67, further comprising providing personalized strategies for mitigating eye strain, including recommended break intervals, adjustments in visual task difficulty, and ergonomic improvements.

Clause 69. The method of any of Clauses 55-68, further comprising implementing safety measures including generating alerts for VR discomfort, providing adjustable field of view settings, and controlling exposure to high-stress visual tasks.

Clause 70. The method of Clause 69, further comprising implementing stress management techniques including micro-breaks and relaxation cues.

Clause 71. The method of any of Clauses 55-70, further comprising: conducting periodic re-evaluations of users to validate the effectiveness of recommended eye strain mitigation strategies; and adjusting the strategies based on the re-evaluation results.

Clause 72. The method of any of Clauses 55-71, wherein the method is adaptable for use in professional settings, including customization for screen-based professionals, engineers, and designers who work with complex visuals, and generation of specific reports for occupational health purposes.

Clause 73. A method of implementing a virtual reality (VR) system for evaluating vision during digital device use and identifying blue light sensitivity, comprising: at an electronic device including a high-resolution VR headset with eye-tracking sensors: generating a VR user interface simulating digital device use; rendering the VR user interface on the VR headset; presenting a series of digital tasks in the VR environment; simulating blue light exposure during the digital tasks; continuously monitoring eye movements and behavior during the tasks; and evaluating the monitored data for indicators of blue light sensitivity.

Clause 74. The method of any of Clauses 73, wherein the high-resolution VR headset has a resolution of at least 60 pixels per degree (PPD), is calibrated to simulate blue light spectra accurately, and can replicate blue light wavelengths of 400-490 nm at various intensities.

Clause 75. The method of any of Clauses 73 or 74, wherein presenting a series of digital tasks comprises simulating real-world patterns of digital device use for sessions lasting 1-4 hours, either continuously or spread throughout the day.

Clause 76. The method of any of Clauses 73-75, wherein the digital tasks include reading varying text sizes, navigating websites, and interacting with interfaces, with gradual increases in task difficulty.

Clause 77. The method of Clause 76, wherein increasing task difficulty comprises reducing font size or increasing screen brightness over time.

Clause 78. The method of any of Clauses 73-77, wherein evaluating the monitored data comprises: assessing blink rate, with decreasing rates suggesting potential sensitivity; measuring pupil dilation, with sustained dilation under blue light exposure indicating sensitivity; and analyzing fixation stability, with decreased stability indicating discomfort.

Clause 79. The method of any of Clauses 73-78, further comprising differentiating between general fatigue and blue light sensitivity by analyzing pupil constriction rates and changes in contrast sensitivity under blue light conditions.

Clause 80. The method of any of Clauses 73-79, further comprising providing recommendations based on detected sensitivity, including prioritizing blue light filters or suggesting adjustments to screen brightness.

Clause 81. The method of Clause 80, wherein the recommendations are tailored to the specific nature of the digital task being performed.

Clause 82. The method of any of Clauses 73-81, further comprising simulating different lighting conditions, including day and night conditions, and accounting for natural light fluctuations.

Clause 83. The method of any of Clauses 73-82, further comprising providing personalized strategies for mitigating blue light sensitivity, including: suggesting the use of blue light filters or glasses; recommending lower screen brightness or reduced exposure time; and specifying break intervals based on real-time data.

Clause 84. The method of any of Clauses 73-83, further comprising generating a comprehensive report on blue light sensitivity, including sensitivity metrics, visual fatigue indicators, and environmental conditions.

Clause 85. The method of Clause 84, wherein the comprehensive report is presented through a user-friendly interface with actionable recommendations.

Clause 86. The method of any of Clauses 73-85, further comprising: performing an initial calibration to establish a baseline sensitivity to blue light; and dynamically adjusting the simulation based on user responses in real-time.

Clause 87. The method of any of Clauses 73-86, wherein the eye-tracking sensors have an accuracy within 0.1 mm and a latency of less than 10 ms to detect subtle eye movement changes.

Clause 88. The method of any of Clauses 73-87, further comprising: reassessing blue light sensitivity after implementing recommended strategies; and confirming the effectiveness of the strategies based on the reassessment.

Clause 89. The method of any of Clauses 73-88, wherein the method is adaptable for use in professional settings with prolonged screen use, such as offices or design studios.

Clause 90. The method of Clause 89, further comprising generating customizable reports for occupational health needs.

Clause 91. A method of implementing a virtual reality (VR) system for testing cognitive load and mental fatigue effects on vision, comprising: at an electronic device including a high-resolution VR headset with eye-tracking sensors: generating a VR user interface simulating high-stress multitasking scenarios; rendering the VR user interface on the VR headset; presenting a series of interactive multitasking scenarios in the VR environment; continuously monitoring eye movements and behavior during the scenarios; and evaluating the monitored data for indicators of cognitive load and mental fatigue.

Clause 92. The method of any of Clauses 91, wherein the high-resolution VR headset has a visual fidelity of at least 60 pixels per degree (PPD) and a responsiveness with latency less than 20 ms.

Clause 93. The method of any of Clauses 91 or 92, wherein presenting a series of interactive multitasking scenarios comprises simulating sessions ranging from 15 to 60 minutes.

Clause 94. The method of any of Clauses 91-93, wherein the interactive multitasking scenarios include: managing data streams while tracking moving objects and solving visual puzzles; and simultaneously controlling multiple virtual instruments while responding to dynamic visual changes.

Clause 95. The method of any of Clauses 91-94, further comprising progressively increasing difficulty and time constraints by gradually increasing the number of tasks and speed of stimuli, while decreasing time allowances for each task.

Clause 96. The method of any of Clauses 91-95, wherein evaluating the monitored data comprises: assessing blink rate, with a drop-in rate signaling high cognitive load; measuring fixation stability, with instability indicating difficulty in maintaining focus; and analyzing saccadic movements, with longer saccades reflecting increased cognitive load.

Clause 97. The method of any of Clauses 91-96, further comprising: performing an initial calibration to establish baseline cognitive and visual performance; and dynamically adjusting task difficulty based on real-time performance metrics.

Clause 98. The method of any of Clauses 91-97, further comprising using one or more algorithms to evaluate cognitive performance by monitoring visual acuity, measuring reaction time, and analyzing error rates.

Clause 99. The method of Clause 98, wherein monitoring visual acuity comprises tracking real-time task performance metrics in the VR environment.

Clause 100. The method of Clause 98, wherein measuring reaction time comprises analyzing the time taken to respond to visual cues presented in the VR scenarios.

Clause 101. The method of Clause 98, wherein analyzing error rates comprises detecting patterns of cognitive overload based on mistakes made during the multitasking scenarios.

Clause 102. The method of any of Clauses 91-101, further comprising generating a comprehensive report including cognitive load indicators, visual performance metrics, and error rates.

Clause 103. The method of Clause 102, wherein the comprehensive report includes graphs, charts, and personalized recommendations for mitigating cognitive fatigue.

Clause 104. The method of any of Clauses 91-103, further comprising providing personalized strategies for mitigating mental fatigue, including tailored breaks, task difficulty adjustments, and ergonomic suggestions based on individual cognitive capacity and task performance.

Clause 105. The method of any of Clauses 91-104, further comprising implementing safety and comfort measures including: providing visual and auditory cues for relaxation; offering adjustable session lengths; presenting break reminders; and allowing for customizable difficulty levels.

Clause 106. The method of any of Clauses 91-105, further comprising: reassessing cognitive load after implementing mitigation strategies; and validating the effectiveness of the strategies based on the reassessment.

Clause 107. The method of any of Clauses 91-106, wherein the method is adaptable for use in high-stress professions, such as air traffic control or financial trading, with scenarios tailored to specific professional environments.

Clause 108. The method of any of Clauses 91-107, further comprising: updating the multitasking scenarios to reflect new research or workplace demands; and allowing for scenario adjustments to fit specific professional contexts.

Clause 109. A method of implementing a virtual reality (VR) system for evaluating visual discomfort in users with eye strain sensitivity, comprising: at an electronic device including a high-resolution VR headset with eye-tracking sensors: generating a VR user interface simulating visually demanding tasks; rendering the VR user interface on the VR headset; presenting a series of interactive scenarios in the VR environment; continuously monitoring eye movements and behavior during the scenarios; and evaluating the monitored data for indicators of eye strain and visual discomfort.

Clause 110. The method of Clause 109, wherein the high-resolution VR headset has a visual fidelity of at least 60 pixels per degree (PPD), adjustable light intensity and color temperature, and a refresh rate of at least 120 Hz.

Clause 111. The method of any of Clauses 109 or 110, wherein presenting a series of interactive scenarios comprises simulating sessions ranging from 15 to 30 minutes, varying based on task complexity.

Clause 112. The method of any of Clauses 109-111, wherein the interactive scenarios include: prolonged reading tasks with varying text sizes and distances; dynamic tracking of fast-moving objects with variable lighting conditions; and tasks designed to mimic real-world scenarios that cause eye strain.

Clause 113. The method of any of Clauses 109-112, wherein evaluating the monitored data comprises: assessing blink rate, with a decline in rate indicating potential strain; measuring pupil dilation, with consistent dilation suggesting discomfort; and analyzing fixation stability, with reduced stability signaling difficulty in focusing.

Clause 114. The method of any of Clauses 109-113, further comprising: performing an initial calibration to establish baseline visual performance; and dynamically adapting task difficulty based on real-time data.

Clause 115. The method of any of Clauses 109-114, further comprising using one or more algorithms to evaluate visual performance by analyzing visual sharpness, measuring reaction time, and detecting fatigue onset through changes in eye-tracking metrics.

Clause 116. The method of any of Clauses 109-115, further comprising generating a comprehensive report including eye strain indicators, visual performance metrics, and environmental factors.

Clause 117. The method of Clause 116, wherein the comprehensive report includes clear visuals and actionable insights tailored for both personal and clinical use.

Clause 118. The method of any of Clauses 109-117, further comprising providing personalized eye-care solutions, including: suggesting specific lens types based on visual performance; recommending adjustments for screen brightness, contrast, and color temperature; and providing recommendations for workspace ergonomic setup.

Clause 119. The method of any of Clauses 109-118, further comprising implementing safety and comfort measures including presenting break reminders, providing relaxation cues, and offering adjustable session lengths.

Clause 120. The method of any of Clauses 109-119, further comprising: tracking improvements over time to ensure effectiveness of personalized solutions; and validating the effectiveness of recommended eye-care solutions through follow-up assessments.

Clause 121. The method of any of Clauses 109-120, wherein the method is adaptable for use in clinical settings, including integration with patient records in optometry practices.

Clause 122. The method of any of Clauses 109-121, wherein the method is customizable for workplace environments with specific visual demands.

Clause 123. The method of any of Clauses 109-122, further comprising updating and expanding the interactive scenarios based on new research findings or emerging eye health concerns.

Clause 124. The method of any of Clauses 109-123, wherein the VR headset includes a wide field of view to simulate real-world movements and visual experiences.

Clause 125. The method of any of Clauses 109-124, further comprising: establishing baseline eye strain sensitivity levels for the user; comparing real-time monitored data to the baseline levels; and initiating personalized interventions when deviations from the baseline exceed predetermined thresholds.

Clause 126. The method of any of Clauses 109-125, further comprising: simulating various environmental conditions that may exacerbate eye strain; assessing the user's sensitivity to these conditions; and providing specific recommendations for managing eye strain in different environments.

Clause 127. A system for implementing a virtual eye test, comprising: a head-mounted display including a display and one or more cameras; one or more processors; and memory storing one or more programs configured to be executed by the one or more processors, the one or more programs including instructions for performing the method of any of Clauses 1-126.

In some embodiments, any of the above clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

### FURTHER CONSIDERATIONS

As used herein, the word “module” refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A method of implementing a virtual reality (VR) system for identifying potential eye strain issues through prolonged engagement, comprising:
at an electronic device including a high-resolution VR headset with eye-tracking sensors:
generating a VR user interface corresponding to a three-dimensional virtual environment;
rendering the VR user interface on the VR headset;
presenting a series of progressively challenging visual tasks in the VR environment;
continuously monitoring eye movements and behavior during the visual tasks; and
evaluating the monitored data for indicators of eye strain.

2. The method of claim 1, wherein the high-resolution VR headset has a resolution of at least 60 pixels per degree (PPD), a refresh rate of 120 Hz, and a field of view of 110 degrees or more.

3. The method of claim 1, wherein the series of visual tasks includes reading fine print for periods exceeding 20 minutes, tracking fast-moving objects for 15-30 minutes, and focus switching tasks for periods over 30 minutes.

4. The method of claim 1, wherein the progressively challenging visual tasks comprise reading documents of varying font sizes, tracking fast-moving objects, and switching focus between near and far objects.

5. The method of claim 4, wherein the tasks start with low complexity and gradually introduce more elements or faster movement to challenge the user.

6. The method of claim 1, wherein evaluating the monitored data comprises:
assessing blink rate, with rates below 10 blinks per minute indicating potential fatigue;
measuring fixation stability, with variations greater than 0.5 degrees suggesting strain; and
analyzing saccade duration, with prolonged saccades indicating increased cognitive load.

7. The method of claim 1, further comprising:
performing an initial calibration to establish baseline visual performance for the user; and
dynamically adapting the difficulty of visual tasks in real-time based on user performance and strain indicators.

8. The method of claim 1, further comprising using one or more algorithms to assess visual acuity, reaction time, and fatigue symptoms.

9. The method of claim 8, wherein assessing visual acuity comprises conducting sharpness and clarity tests in the VR environment.

10. The method of claim 8, wherein measuring reaction time comprises analyzing how quickly users respond to visual stimuli presented in the VR environment.

11. The method of claim 8, wherein detecting fatigue symptoms comprises analyzing changes in blink rate and saccadic patterns over time.

12. The method of claim 1, further comprising generating a comprehensive report including blink rate trends, fixation stability data, saccadic behavior analysis, and visual acuity metrics.

13. The method of claim 12, wherein the comprehensive report includes graphs and actionable insights for both users and clinicians.

14. The method of claim 1, further comprising providing personalized strategies for mitigating eye strain, including recommended break intervals, adjustments in visual task difficulty, and ergonomic improvements.

15. The method of claim 1, further comprising implementing safety measures including generating alerts for VR discomfort, providing adjustable field of view settings, and controlling exposure to high-stress visual tasks.

16. The method of claim 15, further comprising implementing stress management techniques including micro-breaks and relaxation cues.

17. The method of claim 1, further comprising:
conducting periodic re-evaluations of users to validate the effectiveness of recommended eye strain mitigation strategies; and
adjusting the strategies based on the re-evaluation results.

18. The method of claim 1, further comprising, in professional settings, providing customization for screen-based professionals, engineers, and designers who work with complex visuals, and generating one or more reports for occupational health purposes.

19. A system for identifying potential eye strain issues through prolonged engagement, comprising:
a high-resolution VR headset with eye-tracking sensors;
one or more processors; and
memory storing one or more programs configured to be executed by the one or more processors, the one or more programs including instructions for:
generating a VR user interface corresponding to a three-dimensional virtual environment;
rendering the VR user interface on the VR headset;
presenting a series of progressively challenging visual tasks in the VR environment;
continuously monitoring eye movements and behavior during the visual tasks; and
evaluating the monitored data for indicators of eye strain.

20. A non-transitory computer-readable storage medium storing one or more programs configured to be executed by one or more processors of an electronic device including a high-resolution VR headset with eye-tracking sensors, the one or more programs including instructions for:
generating a VR user interface corresponding to a three-dimensional virtual environment;
rendering the VR user interface on the VR headset;
presenting a series of progressively challenging visual tasks in the VR environment;
continuously monitoring eye movements and behavior during the visual tasks; and
evaluating the monitored data for indicators of eye strain.
